(19) United States
(12) Patent Application Publication
(10) Pub. No.: US 2024/0318156 A1
(43) Pub. Date: Sep. 26, 2024

(54) COMPOSITIONS OF BETA-HEXOSAMINIDASE VARIANTS AND USES THEREOF

(71) Applicant: BIOMARIN PHARMACEUTICAL INC., Novato, CA (US)

(72) Inventors: Daniel WENDT, Novato, CA (US); Mika AOYAGI-SCHARBER, Novato, CA (US); Roger LAWRENCE,, Novato, CA (US); Manuel LOPEZ, Novato, CA (US)

(21) Appl. No.: 18/573,298

(22) PCT Filed: Jun. 24, 2022
(86) PCT No.: PCT/US2022/034861

§ 371 Date: Dec. 21, 2023

Related U.S. Application Data
(60) Provisional application No. 63/215,328, filed on Jun. 25, 2021.

(51) Int. Cl. C12N 9/24; A61K 38/00

(52) U.S. Cl. CPC C12N 9/2402; C12Y 302/01052; A61K 38/00

(57) ABSTRACT

Disclosed herein are recombinant β-hexosaminidase variant a subunits that form a β-hexosaminidase variant a subunit homodimer that have optimized properties for use in treating Tay-Sachs disease or Sandhoff Disease.

### CROSS-REFERENCE

The present application claims the benefit of U. S. Provisional Application No. 63/215,328, filed on Jun. 25, 2021, which is incorporated herein by reference in its entirety.

### SUMMARY

Disclosed herein are recombinant β-hexosaminidase variant a subunits that form a β-hexosaminidase variant α subunit homodimer comprising one or more amino acid sequence substitutions or deletions at positions corresponding to S184, P209, N228, P229, V230, T231, P429, K432, D433, 1436, N466, S491, L493, T494, F495, E498, L508, Q513, N518, V519, F521, and E523 of native β-hexosaminidase α subunit of SEQ ID NO:1 and further comprising one or more amino acid sequence elements that increase cellular uptake of the β-hexosaminidase variant α subunit homodimer relative to a homodimer of SEQ ID NO: 6. In some embodiments, the one or more amino acid sequence elements that increase cellular uptake are selected from the group consisting of amino acid sequence substitutions, additions, or deletions relative to the native β-hexosaminidase α subunit of SEQ ID NO:1. In some embodiments, the β-hexosaminidase variant α subunit comprises one or more amino acid substitutions or deletions corresponding to S184K, P209Q, N228S, P229Δ, V230L, T231S, P429Q, K432R, D433K, 1436K, N466A, S491R, L493M, T494D, F495D, E498D, L508V, Q513V, N518Y, V519A, F521Y, and E523N relative to the native β-hexosaminidase α subunit of SEQ ID NO: 1. In some embodiments, the β-hexosaminidase variant α subunit comprises at least five amino acid substitutions or deletions corresponding to S184K, P209Q, N228S, P2294, V230L, T231S, P429Q, K432R, D433K, 1436K, N466A, S491R, L493M, T494D, F495D, E498D, L508V, Q513V, N518Y, V519A, F521Y, and E523N relative to the native β-hexosaminidase α subunit of SEQ ID NO:1. In some embodiments, the β-hexosaminidase variant α subunit comprises at least ten amino acid substitutions or deletions corresponding to S184K, P209Q, N228S, P229Δ, V230L, T231S, P429Q, K432R, D433K, I436K, N466A, S491R, L493M, T494D, F495D, E498D, L508V, Q513V, N518Y, V519A, F521Y, and E523N relative to the native β-hexosaminidase α subunit of SEQ ID NO: 1. In some embodiments, the β-hexosaminidase variant α subunit comprises at least fifteen amino acid substitutions or deletions corresponding to S184K, P209Q, N228S, P229Δ, V230L, T231S, P429Q, K432R, D433K, 1436K, N466A, S491R, L493M, T494D, F495D, E498D, L508V, Q513V, N518Y, V519A, F521Y, and E523N relative to the native β-hexosaminidase α subunit of SEQ ID NO:1. In some embodiments, the β-hexosaminidase variant α subunit comprises at least twenty amino acid substitutions or deletions corresponding to S184K, P209Q, N228S, P229Δ, V230L, T231S, P429Q, K432R, D433K, 1436K, N466A, S491R, L493M, T494D, F495D, E498D, L508V, Q513V, N518Y, V519A, F521Y, and E523N relative to the native β-hexosaminidase α subunit of SEQ ID NO:1. In some embodiments, the β-hexosaminidase variant α subunit comprises amino acid substitutions or deletions corresponding to S184K, P209Q, N228S, P229Δ, V230L, T231S, P429Q, K432R, D433K, 1436K, N466A, S491R, L493M, T494D, F495D, E498D, L508V, Q513V, N518Y, V519A, F521Y, and E523N relative to the native β-hexosaminidase α subunit of SEQ ID NO:1. In some embodiments, the β-hexosaminidase variant α subunit comprises a first amino acid sequence comprising at least 85% sequence identity to SEQ ID NO: 11 and the one or more amino acid sequence elements that increase cellular uptake of the β-hexosaminidase variant α subunit relative to a homodimer of SEQ ID NO: 6 comprises a second amino acid sequence. In some embodiments, the first amino acid sequence comprises at least 90% sequence identity to SEQ ID NO: 11. In some embodiments, the first amino acid sequence comprises at least 95% sequence identity to SEQ ID NO: 11. In some embodiments, the first amino acid sequence comprises at least 99% sequence identity to SEQ ID NO: 11. In some embodiments, the first amino acid sequence comprises the amino acid sequence of SEQ ID NO: 11. In some embodiments, the second amino acid sequence is N-terminal to the first amino acid sequence. In some embodiments, the second amino acid sequence comprises at least 20 contiguous amino acid residues of SEQ ID NO: 2. In some embodiments, the second amino acid sequence comprises at least 100 contiguous amino acid residues of SEQ ID NO: 12. In some embodiments, the second amino acid sequence comprises at least 100 contiguous amino acid residues of SEQ ID NO: 13. In some embodiments, the second amino acid sequence comprises at least 150 contiguous amino acid residues of SEQ ID NO: 13. In some embodiments, the second amino acid sequence comprises at least 85% sequence identity to SEQ ID NO: 13. In some embodiments, the second amino acid sequence comprises at least 90% sequence identity to SEQ ID NO: 13. In some embodiments, the second amino acid sequence comprises at least 95% sequence identity to SEQ ID NO: 13. In some embodiments, the second amino acid sequence comprises the amino acid sequence of SEQ ID NO: 13. In some embodiments, the β-hexosaminidase variant α subunit comprises an amino acid sequence with at least 90% sequence identity to SEQ ID NO: 3. In some embodiments, the β-hexosaminidase variant α subunit comprises an amino acid sequence with at least 95% sequence identity to SEQ ID NO: 3. In some embodiments, the β-hexosaminidase variant α subunit comprises an amino acid sequence with at least 99% sequence identity to SEQ ID NO: 3. In some embodiments, the (3-hexosaminidase variant α subunit comprises the amino acid sequence according to SEQ ID NO: 3. In some embodiments, the β-hexosaminidase variant α subunit comprises at least 500 contiguous amino acids of an amino acid sequence according to SEQ ID NO: 3. In some embodiments, the (3-hexosaminidase variant α subunit comprises at least 525 contiguous amino acids of an amino acid sequence according to SEQ ID NO: 3. In some embodiments, the β-hexosaminidase variant α subunit comprises at least 550 contiguous amino acids of an amino acid sequence according to SEQ ID NO: 3. In some embodiments, the β-hexosaminidase variant α subunit comprises at least 500 contiguous amino acids of an amino acid sequence according to SEQ ID NO: 3, and at least 95% sequence identity to the at least 500 contiguous amino acids. In some embodiments, the β-hexosaminidase variant α subunit comprises at least 525 contiguous amino acids of an amino acid sequence according to SEQ ID NO: 3, and at least 95% sequence identity to the at least 525 contiguous amino acids. In some embodiments, the β-hexosaminidase variant α subunit comprises at least 550 contiguous amino acids of an amino acid sequence according to SEQ ID NO: 3, and at least 95% sequence identity to the at least 550 contiguous amino acids. In some embodiments, the β-hexosaminidase variant α subunit comprises an amino acid sequence with at least 85% sequence identity to SEQ ID NO: 3. In some embodiments, the β-hexosaminidase variant α subunit comprises an increased mannose-6-phosphorylation (M6P) relative to a homodimer of SEQ ID NO: 6. In some embodiments, the β-hexosaminidase variant α subunit homodimer has at least 3 M6P sites occupied by M6P per homodimer. In some embodiments, the β-hexosaminidase variant α subunit homodimer has at least 4 M6P sites occupied by M6P per homodimer. In some embodiments, the β-hexosaminidase variant α subunit homodimer exhibits GM2 ganglioside hydrolysis activity in the presence of GM2-activator protein. In some embodiments, the cellular uptake of the β-hexosaminidase variant α subunit homodimer is increased by at least 2-fold, 5-fold, 10-fold, 20-fold, or 25-fold relative to a homodimer of SEQ ID NO: 6. In some embodiments, the cellular uptake of the variant the β-hexosaminidase variant α subunit homodimer is assessed by uptake of cation independent mannose-6-phosphate receptor (CI-MPR) in a competition assay of the homodimer and mannose-6-phosphate in human fibroblasts from SD patients. In some embodiments, the β-hexosaminidase variant α subunit homodimer has increased thermal stability relative to a heterodimer of SEQ ID NO: 1 and SEQ ID NO: 2. In some embodiments, the β-hexosaminidase variant α subunit homodimer has a melting point (Tm) of about 60° C. to about 63° C. In some embodiments, the β-hexosaminidase variant α subunit homodimer increases lifespan of a Hexb knockout mouse when administered at least age 80 days of age as compared to a heterodimer of SEQ ID NO: 1 and SEQ ID NO: 2 that is administered to a Hexb knockout mouse under substantially equivalent assay conditions and age. In some embodiments, the β-hexosaminidase variant α subunit homodimer increases lifespan by at least 2-fold of a Hexb knockout mouse when administered at least 80 days of age as compared to a heterodimer of SEQ ID NO: 1 and SEQ ID NO: 2 that is administered to a Hexb knockout mouse under substantially equivalent assay conditions and age.

Disclosed herein are homodimers comprising the recombinant β-hexosaminidase variant α subunit according to any one of the above embodiments.

Disclosed herein are pharmaceutical compositions comprising the homodimer according to any of the above embodiments, and one or more pharmaceutically acceptable excipients. In some embodiments, the one or more pharmaceutically acceptable excipients comprise sodium phosphate, sodium chloride, potassium chloride, magnesium chloride, and calcium chloride. In some embodiments, the pharmaceutical composition comprises 1 mM sodium phosphate, 148 sodium chloride, 3 mM potassium chloride, 0.8 mM magnesium chloride, 1.4 mM calcium chloride, pH 7.2. In some embodiments, the homodimer comprising the recombinant β-hexosaminidase variant α subunit is formulated at a concentration of about 20 mg/ml.

Disclosed herein are methods of ameliorating the symptoms or slowing progression of TSD in a subject having TSD comprising administering to the subject an effective amount of the pharmaceutical composition of any one of the above embodiments. Disclosed herein are methods of ameliorating the symptoms or slowing progression of SD in a subject having SD comprising administering to the subject an effective amount of the pharmaceutical composition of any one of the above embodiments. In some embodiments, the pharmaceutical composition is administered to the subject intracerebroventricularly. In some embodiments, the pharmaceutical composition is administered to the subject intracerebroventricularly weekly. In some embodiments, the pharmaceutical composition is administered every other week. In some embodiments, the pharmaceutical composition is administered at regular intervals, wherein the regular intervals are greater than every other week. In some embodiments, the subject is pre-symptomatic for TSD. In some embodiments, the subject has a TSD-associated mutation. In some embodiments, the subject has an elevated level of a biomarker of TSD relative to a control level of the biomarker of TSD. In some embodiments, the elevated level of a biomarker of TSD is a measurement of the biomarker of TSD obtained from a sample from the subject. In some embodiments, the control level of the biomarker of TSD is a measurement of the biomarker of TSD from a sample from a subject that does not have a TSD-associated mutation. In some embodiments, the sample from the subject and the sample from the subject that does not have a TSD-associated mutation are quantified using liquid chromatography and mass spectrometry. In some embodiments, the sample from the subject and the sample from the subject that does not have a TSD-associated mutation are obtained from the CSF or blood. In some embodiments, the sample from the subject and the sample from the subject that does not have a TSD-associated mutation are blood plasma samples. In some embodiments, the elevated level of the biomarker of the TSD is elevated by at least 100% to 4000% relative to the control level of the biomarker of TSD. In some embodiments, the elevated level of the biomarker of the TSD is elevated by at least 2× to 100× relative to the control level of the biomarker of TSD. In some embodiments, the biomarker is GM2, GA2, A2G0′ containing beta-linked terminal N-acetyl-D-hexosamine, BMP(22:6) phospholipid (Bis[monoacylglycero]phosphate)), neurofilament light chain or a free glycan. In some embodiments, the subject is pre-symptomatic for SD. In some embodiments, the subject has a SD-associated mutation. In some embodiments, the subject has an elevated level of a biomarker of SD relative to a control level of the biomarker of SD. In some embodiments, the elevated level of a biomarker of SD is a measurement of the biomarker of SD obtained from a sample from the subject. In some embodiments, the control level of the biomarker of SD is a measurement of the biomarker of SD from a sample from a subject that does not have a SD-associated mutation. In some embodiments, the sample from the subject and the sample from the subject that does not have a SD-associated mutation are quantified using liquid chromatography and mass spectrometry. In some embodiments, the sample from the subject and the sample from the subject that does not have a SD-associated mutation are obtained from the CSF or blood. In some embodiments, the sample from the subject and the sample from the subject that does not have a SD-associated mutation are blood plasma samples. In some embodiments, the elevated level of the biomarker of the SD is elevated by at least 100% to 4000% relative to the control level of the biomarker of SD. In some embodiments, the elevated level of the biomarker of the SD is elevated by at least 2× to 100× relative to the control level of the biomarker of SD. In some embodiments, the biomarker is GM2, GA2, A2G0′ containing beta-linked terminal N-acetyl-D-hexosamine, BMP(22:6) phospholipid (Bis[monoacylglycero]phosphate)), neurofilament light chain or a free glycan.

Disclosed herein are nucleic acids encoding the homodimer comprising the recombinant β-hexosaminidase variant α subunit of any of the above embodiments. Disclosed herein are vectors comprising the nucleic acid of any of the above embodiments and one or more gene regulatory regions. In some embodiments, the vector further comprises adeno-associated virus (AAV) inverted terminal repeats (ITR) at both the 5′ and 3′ end of the vector. Disclosed herein are AAV viral particles comprising the vector of the above embodiments. Disclosed herein are methods of ameliorating the symptoms or slowing disease progression of TSD in a subject having TSD comprising administering an effective amount of the AAV viral particle of any of the above embodiments to the subject having TSD. Disclosed herein are methods of ameliorating the symptoms or slowing disease progression of SD in a subject having TSD comprising administering an effective amount of the AAV viral particle of any of the above embodiments to the subject having SD. Disclosed herein are methods of reducing GM2 ganglioside accumulation in a subject having TSD or SD comprising administering an effective amount of the pharmaceutical composition of any of the above embodiments to a subject having TSD or SD. Disclosed herein are methods of reducing the level of a biomarker of disease progression of TSD in a subject having TSD comprising administering an effective amount of the pharmaceutical composition of any of the above embodiments to a subject having TSD. Disclosed herein are methods of reducing the level of a biomarker of disease progression of TSD in a subject having TSD comprising administering an effective amount of the AAV viral particle of any of the above embodiments to a subject having TSD. Disclosed herein are methods of reducing the level of a biomarker of disease progression of SD in a subject having SD comprising administering an effective amount of the pharmaceutical composition of any of the above embodiments to a subject having or SD. Disclosed herein are methods of reducing the level of a biomarker of disease progression of SD in a subject having SD comprising administering an effective amount of the viral particle of any of the above embodiments to a subject having or SD. In some embodiments, the biomarker of disease progression of TSD or SD is a GM2 and GA2, A2G0′ containing beta-linked terminal N-acetyl-D-hexosamine, BMP(22:6) phospholipid (Bis[monoacylglycero]phosphate)) or neurofilament light chain or a free glycan.

### BRIEF DESCRIPTION OF THE DRAWINGS

FIGS. 1A-1C. FIG. 1A provides the HexD3 model based on crystal structures of HexA (PDB ID: 2GJX) and HexB (PDB ID: 1NOU) and the functional domains of HexD3.

FIG. 1B demonstrates that the modified α subunit of HexM was modified to retain a R subunit mannose 6-phosphorylation site by swapping domain I of the α subunit with domain I of the β subunit. The resulting chimeric α3′ subunit contains phosphorylated glycans at position N84 compared to the glycosylated, but not phosphorylated, S51N/A53T site in subunit u. Two additional R glycosylation sites, N190/N142, were also included.

FIG. 1C shows a radar plot comparing the functional attributes of HexA, HexD3, and HexM proteins on a relative scale.

FIG. 2 is a graph showing the saturation kinetics of GM2AP-dependent GM2-degradation activity measured for variant hexosaminidase enzymes.

FIG. 3A is a graph showing the determination of the Kᵤₚₜₐₖₑ (a concentration for half-maximal cellular uptake) value of the HexA, HexM, and Mod2B hexosaminidase variants in Sandhoff (SD) patient-derived fibroblasts.

FIG. 3B is a graph showing the determination of the Kᵤₚₜₐₖₑ (a concentration for half-maximal cellular uptake) value of the HexA, HexM, and HexD3 hexosaminidase variants.

FIG. 4 is a graph showing the inhibition of cellular uptake by mannose-6-phosphate (M6P) of various hexosaminidase variants in Sandhoff patient-derived fibroblasts.

FIGS. 5A-5B are a pair of graphs showing cellular stability, expressed as a half-life of enzyme activity measured in cells. FIG. 5A provides the data for HexA, HexM, and Mod2B in Sandhoff fibroblasts.

FIG. 5B provides the data for HexA and HexD3 in Sandhoff (SD) and Tay-Sachs (TS) fibroblasts.

FIGS. 6A-6C are a set of graphs showing HexD3 activity 24 hours after the final intracerebroventricular (ICV) dose measured using two fluorogenic substrates (4-MUG, 4-methylumbelliferyl-N-acetyl-β-D-glucosaminide; 4-MUGS, 4-methylumbelliferyl-6-sulfo-N-acetyl-β-D-glucosaminide) in brain homogenate from HexD3-treated Sandhoff (KO-HexD3) mice.

FIG. 6A shows that average HexD3 activity levels towards 4-MUG in HexD3 treated Sandhoff mice are similar to hexosaminidase activity levels in wild type mice (WT, dashed line).

FIG. 6B shows that average HexD3 activity levels towards 4-MUGS in HexD3 treated Sandhoff mice exceed hexosaminidase activity levels in wild type mice (WT).

FIG. 6C shows the quantity of HexD3 per mg of brain protein in ICV-HexD3 treated Sandhoff mice.

FIG. 7 is a graph showing the correlation between HexD3 protein levels determined by mass spectrometry quantification and specific enzyme activity measurement using 4-MU-substrate in the brains of Sandhoff mice after ICV administration of HexD3 enzyme.

FIG. 8 is a set of photomicrographs of mouse hippocampus sections from wild-type (WT), vehicle treated SD (Hexβ^{-/-}), and Sandhoff mice treated with HexD3.

FIGS. 9A-9G demonstrate CNS localization and peripheral lysosomal correction following Hex delivery by ICV.

FIG. 9A shows a graphic representation of the anatomical brain distribution of a Hex enzyme.

FIG. 9B shows naphthol signal indicating the biodistribution pattern after 3 q.wk. doses of HexA enzyme.

FIG. 9C shows LAMP2 quantification following 3 q. wk. doses of HexA or HexD3 in various brain regions of interest (ROI).

FIG. 9D shows the mass spectrometry quantitation of the whole brain hemisphere following Hex ICV dosing.

FIG. 9E shows the overlay of NeuN and Naphthol signal to indicate localization of Hex variants to neurons in the hippocampus as well as relative concentrations of protein in tissue.

FIG. 9F shows HexA or HexD3 activity after 7 q.wk. doses in the liver through naphthol staining signal.

FIG. 9G demonstrates liver LAMP2 levels after 3-7 doses of Hex enzyme ICV in Hexβ−/− KO administered HexA, HexD3 or vehicle.

FIGS. 10A-10E show the effect of ICV delivery of HexD3 enzyme in a Hexβ−/− KO mouse model of SD.

FIG. 10A shows a schematic of the study where 3 doses of HexA or HexD3 were administered, at a frequency of one dose every week (q.wk), for 2 weeks starting 2 weeks after cannulation.

FIG. 10B shows HexA and HexD3 enzyme activity levels found in the brain Hexβ−/− KO mouse model of SD.

FIG. 10C shows BMP (22:6) levels in the brain of Hexβ−/− KO mouse model of SD.

FIGS. 10D and 10E show N-linked glycans and GM2:GA2 gangliosides levels in the brain of Hexβ−/− KO mouse model of SD.

FIGS. 11A-11F demonstrates the effects HexD3 ICV on late stage disease progression in a Hexβ−/− KO mouse model of SD.

FIG. 11A is a schematic showing ages when ICV treatment was started in relation to the targeted stages of disease progression.

FIG. 11B shows survival curves of treatment groups administered HexA or HexD3 at different time points.

FIG. 11C shows weight curves of Hexβ−/− KO mouse model of SD undergoing different treatments of HexA or HexD3.

FIG. 11D provides data for nest building, which was used as an indicator of sustained health of animals treated at 56 days or 84 days.

FIG. 11E shows open field locomotor activity analysis.

FIG. 11F demonstrates the effect on motor function loss after HexD3 treatment in a Hexβ−/− KO mouse model of SD.

FIGS. 12A-12C show the effect on blood neurofilament light chain (NF-L) levels with HexA or HexD3 administration Hexβ−/− KO mouse (KO) or Hexβ+/− mouse (Het).

FIG. 12A shows an experimental schematic showing that vehicle and HexA or HexD3 treated animals were injected weekly starting from 56 days of age.

FIG. 12B shows that Hex treatment maintained plasma NF-L at levels below what would be expected in non-treated 84-day or 98-day-old KO mice (dotted lines).

FIG. 12C demonstrates the level of blood NF-L levels with HexA or HexD3 administration.

FIGS. 13A-13C demonstrate the timing of disease progression and the window of therapeutic rescue in a mouse model of SD.

FIG. 13A shows the profile of plasma NF-L levels (left y-axis) superimposed onto locomotor activity (right y-axis) to indicate the age at which the transitions between disease stages (arrows) occur. A schematic of the progressive neurodegeneration in SD mice broken into 4 stages.

FIG. 13B shows a graph depicting the degree of survival benefit compared to the age of treatment when HexA gene therapy was started. Dotted line is an extrapolated linear regression fit through the median survival ages (black squares).

FIGS. 14A-14B show that HexD3 provides a disease rescue potential.

FIG. 14A demonstrates the degree of benefit observed with either recombinant HexA ICV treatment or HexA gene therapy in an SD mouse model.

FIG. 14B shows the modeling behavior of HexA and HexD3 treatments for assessing its activities in GM2 treatment.

FIG. 15 demonstrates loss of NF-L expression in Sandhoff mice reaching euthanasia criteria.

FIG. 16 shows hexosaminidase isoenzyme N-Glycan profiles.

FIGS. 17A-17B demonstrate HexD3 thermostability.

FIG. 17A shows that thermal stability is consistent among different lots of HexA and HexD3.

FIG. 17B demonstrates stability in CSF measured by remaining MUG activity.

FIG. 18 demonstrates the cellular half-life of Hex isozymes.

### DETAILED DESCRIPTION

Tay-Sachs disease (TSD) and Sandhoff Disease (SD) are lysosomal storage diseases (LSD) that are caused by mutations in various lysosomal resident enzymes. Loss of enzyme activity causes the build-up of the substrate of enzyme, leading to symptomatic disease. Enzyme replacement therapy (ERT) has emerged as a viable method of treating LSD. ERT is based on treating an LSD patient with a recombinant form of the missing lysosomal enzyme that is able to be taken up by the patient's cells and delivered to the lysosomal compartment thereby restoring the missing enzyme activity.

One such lysosomal enzyme is β-hexosaminidase, which comprises two subunits (α and β) as either heterodimers or homodimers. β-hexosaminidase catalyzes the degradation of GM2 ganglioside (in a heterodimer form with the GM2 activator protein), and other molecules containing terminal N-acetyl hexosamines. Loss of either the α or β subunit of β-hexosaminidase results in the accumulation of lysosomal GM2, which eventually triggers neuronal cell death. This neuronal cell death causes TSD (caused by mutations in the gene encoding the a subunit) and SD (caused by mutations in the gene encoding the β-subunit).

HexA is a heterodimer of the α- and β-subunits of β-hexosaminidase. HexA, in combination with the GM2-activator protein (GM2AP), converts GM2 to GM3 ganglioside through the hydrolysis of the non-reducing terminal β-linked, N-acetyl-galactosamine residue of GM2. While HexA has been used as a potential therapeutic for TSD and SD, the efficacy of HexA has been low due to the heterodimeric nature of HexA. HexA poses an expression challenge as the α and β subunits are required to dimerize in order for the enzyme to possess all the residues critical for GM2 substrate recognition, GM2AP binding, cellular uptake, and lysosomal targeting. HexA has a theoretical isoelectric point (pI) of 5.52 and a theoretical molecular weight (MW) of 117147.96 Da.

HexB is a β-hexosaminidase enzyme comprising a homodimer of β subunits. While HexB is the most stable dimeric form of β-hexosaminidase, HexB lacks the functional residues required for GM2 hydrolysis, and therefore is not useful for ERT.

HexM is a β-hexosaminidase enzyme comprising a modified α subunit homodimer. In particular, HexM is made of 2 a subunits having the following amino acid substitutions or deletions corresponding to S184K, P209Q, N228S, P229Δ, V230L, T231S, P429Q, K432R, D433K, 1436K, N466A, S491R, L493M, T494D, F495D, E498D, L508V, Q513V, N518Y, V519A, F521Y, and E523N of the native β-hexosaminidase α subunit of SEQ ID NO:1. HexM has greater activity and stability but has poor cellular uptake in vitro compared to HexA.

### Recombinant β-Hexosaminidase Variant α Subunit Compositions

Disclosed herein are β-hexosaminidase variant α subunit compositions that have been optimized for improved properties for use in treating TSD and SD. In some embodiments, the 1-hexosaminidase variant α subunit described herein form β-hexosaminidase variant α subunit homodimers that have improved properties for use in treating TSD and SD relative to HexA, HexM, HexB, or Mod2B. In some embodiments, the β-hexosaminidase variant α subunit described herein form β-hexosaminidase variant α subunit homodimers that have improved properties for use in treating TSD and SD relative to HexA or HexM. In some embodiments, the β-hexosaminidase variant α subunit described herein form β-hexosaminidase variant α subunit homodimers that have improved properties for use in treating TSD and SD relative to HexA and HexM. In some embodiments, the β-hexosaminidase variant α subunit described herein form β-hexosaminidase variant α subunit homodimers that have improved properties for use in treating TSD and SD relative to HexA. In some embodiments, the β-hexosaminidase variant α subunit described herein form β-hexosaminidase variant α subunit homodimers that have improved properties for use in treating TSD and SD relative to HexM.

In some embodiments, a β-hexosaminidase variant α subunit of the disclosure comprises an amino acid change relative to the HexA a subunit, which can convert the dimer interface from a to R and introduce the putative GM2AP binding domain from β onto the α subunit. A variant as disclosed herein can be a polypeptide that comprises one or more differences in the amino acid sequence of the variant relative to a natural occurring reference sequence. A variant can include, for example, a deletion, addition, or substitution of an amino acid residue relative to a reference sequence. In some embodiments, a β-hexosaminidase variant α subunit described herein forms a homodimer. In some embodiments, a β-hexosaminidase variant α subunit described herein forms a heterodimer. In some embodiments, a β-hexosaminidase variant α subunit described herein hydrolyzes GM2 ganglioside.

Examples of amino acid residue changes that can be incorporated in a β-hexosaminidase variant α subunit disclosed herein are shown in TABLE 1. The amino acid residue changes can be, for example, mutations, substitutions, deletions, or additions.

TABLE 1
Example amino acid residue changes for β-hexosaminidase
variant α subunits

| Residue Position | | |
|---|---|---|
| (α numbering | | |
| corresponding to | Change | |
| SEQ ID NO: 1) | (α to β) | Effect |
| 184 | Ser (S) to Lys (K) | β dimer interface |
| 209 | Pro (P) to Gln (Q) | β dimer interface |
| 228 | Asn (N) to Ser (S) | β dimer interface |
| 229 | Pro deletion | β dimer interface |
| 230 | Val (V) to Leu (L) | β dimer interface |
| 231 | Thr (T) to Ser (S) | β dimer interface |
| 429 | Pro (P) to Gln (Q) | β dimer interface and |
| | | GM2A binding site |
| 432 | Lys (K) to Arg (R) | GM2A binding site |
| 433 | Asp (D) to Lys (K) | GM2A binding site |
| 436 | Ile (I) or Val (V) | GM2A binding site |
| | to Lys (K) | |
| 466 | Asn (N) to Ala (A) | β dimer interface |
| 491 | Ser (S) to Arg (R) | GM2A binding site |
| 493 | Leu (L) to Met (M) | GM2A binding site |
| 494 | Thr (T) to Asp (D) | GM2A binding site |
| 495 | Phe (F) to Asp (D) | GM2A binding site |
| 498 | Glu (E) to Asp (D) | GM2A binding site |
| 508 | Leu (L) to Val (V) | β dimer interface |
| 513 | Gln (Q) to Ala (A) | β dimer interface |
| 518 | Asn (N) to Tyr (Y) | β dimer interface |
| 519 | Val (V) to Ala (A) | β dimer interface |
| 521 | Phe (F) to Tyr (Y) | β dimer interface |
| 523 | Glu (E) to Asn (N) | β dimer interface |

Disclosed herein are recombinant β-hexosaminidase variant α subunits that form a β-hexosaminidase variant α subunit homodimer comprising one or more amino acid sequence substitutions or deletions at positions corresponding to S184, P209, N228, P229, V230, T231, P429, K432, D433, 1436, N466, 5491, L493, T494, F495, E498, L508, Q513, N518, V519, F521, and E523 of native β-hexosaminidase α subunit of SEQ ID NO:1 and further comprising one or more amino acid sequence elements that increase cellular uptake of the β-hexosaminidase variant α subunit homodimer relative to a homodimer of SEQ ID NO: 6. In some embodiments, the one or more amino acid sequence elements that increase cellular uptake are selected from the group consisting of amino acid sequence substitutions, additions, or deletions relative to the native β-hexosaminidase α subunit of SEQ ID NO:1.

In some embodiments, the β-hexosaminidase variant α subunit comprises one or more amino acid substitutions or deletions corresponding to S184K, P209Q, N228S, P229Δ, V230L, T231S, P429Q, K432R, D433K, 1436K, N466A, S491R, L493M, T494D, F495D, E498D, L508V, Q513V, N518Y, V519A, F521Y, and E523N relative to the native β-hexosaminidase α subunit of SEQ ID NO:1. In some embodiments, the β-hexosaminidase variant α subunit comprises at least five amino acid substitutions or deletions corresponding to S184K, P209Q, N228S, P229Δ, V230L, T231S, P429Q, K432R, D433K, 1436K, N466A, S491R, L493M, T494D, F495D, E498D, L508V, Q513V, N518Y, V519A, F521Y, and E523N relative to the native β-hexosaminidase α subunit of SEQ ID NO:1. In some embodiments, the β-hexosaminidase variant α subunit comprises at least ten amino acid substitutions or deletions corresponding to S184K, P209Q, N228S, P229Δ, V230L, T231S, P429Q, K432R, D433K, 1436K, N466A, S491R, L493M, T494D, F495D, E498D, L508V, Q513V, N518Y, V519A, F521Y, and E523N relative to the native β-hexosaminidase α subunit of SEQ ID NO:1. In some embodiments, the β-hexosaminidase variant α subunit comprises at least fifteen amino acid substitutions or deletions corresponding to S184K, P209Q, N228S, P229Δ, V230L, T231S, P429Q, K432R, D433K, 1436K, N466A, S491R, L493M, T494D, F495D, E498D, L508V, Q513V, N518Y, V519A, F521Y, and E523N relative to the native β-hexosaminidase α subunit of SEQ ID NO:1. In some embodiments, the β-hexosaminidase variant α subunit comprises at least twenty amino acid substitutions or deletions corresponding to S184K, P209Q, N228S, P229Δ, V230L, T231S, P429Q, K432R, D433K, 1436K, N466A, S491R, L493M, T494D, F495D, E498D, L508V, Q513V, N518Y, V519A, F521Y, and E523N relative to the native β-hexosaminidase α subunit of SEQ ID NO:1. In some embodiments, the β-hexosaminidase variant α subunit comprises amino acid substitutions or deletions corresponding to S184K, P209Q, N228S, P229Δ, V230L, T231S, P429Q, K432R, D433K, 1436K, N466A, S491R, L493M, T494D, F495D, E498D, L508V, Q513V, N518Y, V519A, F521Y, and E523N relative to the native β-hexosaminidase α subunit of SEQ ID NO: 1.

A sequence of a recombinant β-hexosaminidase variant α subunit disclosed herein can have at least about 70% homology, at least about 71% homology, at least about 72% homology, at least about 73% homology, at least about 74% homology, at least about 75% homology, at least about 76% homology, at least about 77% homology, at least about 78% homology, at least about 79% homology, at least about 80% homology, at least about 81% homology, at least about 82% homology, at least about 83% homology, at least about 84% homology, at least about 85% homology, at least about 86% homology, at least about 87% homology, at least about 88% homology, at least about 89% homology, at least about 90% homology, at least about 91% homology, at least about 92% homology, at least about 93% homology, at least about 94% homology, at least about 95% homology, at least about 96% homology, at least about 97% homology, at least about 98% homology, or at least about 99% homology to an amino acid sequence provided herein.

A sequence of a recombinant β-hexosaminidase variant α subunit disclosed herein can have at least about 70% identity, at least about 71% identity, at least about 72% identity, at least about 73% identity, at least about 74% identity, at least about 75% identity, at least about 76% identity, at least about 77% identity, at least about 78% identity, at least about 79% identity, at least about 80% identity, at least about 81% identity, at least about 82% identity, at least about 83% identity, at least about 84% identity, at least about 85% identity, at least about 86% identity, at least about 87% identity, at least about 88% identity, at least about 89% identity, at least about 90% identity, at least about 91% identity, at least about 92% identity, at least about 93% identity, at least about 94% identity, at least about 95% identity, at least about 96% identity, at least about 97% identity, at least about 98% identity, or at least about 99% identity to an amino acid sequence provided herein. In some embodiments, a variant β-hexosaminidase enzyme disclosed herein comprises an amino acid sequence having 100% identity to SEQ ID NO: 3.

Various methods and software programs can be used to determine the homology between two or sequences, such as NCBI BLAST, Clustal W, MAFFT, Clustal Omega, AlignMe, Praline, or another suitable method or algorithm.

In some embodiments, the β-hexosaminidase variant α subunit comprises a first amino acid sequence comprising at least 70% sequence identity to SEQ ID NO: 11 and the one or more amino acid sequence elements that increase cellular uptake of the β-hexosaminidase variant α subunit relative to a homodimer of SEQ ID NO: 6 comprises a second amino acid sequence. In some embodiments, the β-hexosaminidase variant α subunit comprises a first amino acid sequence comprising at least 75% sequence identity to SEQ ID NO: 11 and the one or more amino acid sequence elements that increase cellular uptake of the β-hexosaminidase variant α subunit relative to a homodimer of SEQ ID NO: 6 comprises a second amino acid sequence. In some embodiments, the β-hexosaminidase variant α subunit comprises a first amino acid sequence comprising at least 80% sequence identity to SEQ ID NO: 11 and the one or more amino acid sequence elements that increase cellular uptake of the β-hexosaminidase variant α subunit relative to a homodimer of SEQ ID NO: 6 comprises a second amino acid sequence. In some embodiments, the β-hexosaminidase variant α subunit comprises a first amino acid sequence comprising at least 85% sequence identity to SEQ ID NO: 11 and the one or more amino acid sequence elements that increase cellular uptake of the β-hexosaminidase variant α subunit relative to a homodimer of SEQ ID NO: 6 comprises a second amino acid sequence. In some embodiments, the first amino acid sequence comprises at least 90% sequence identity to SEQ ID NO: 11. In some embodiments, the first amino acid sequence comprises at least 91% sequence identity to SEQ ID NO: 11. In some embodiments, the first amino acid sequence comprises at least 92% sequence identity to SEQ ID NO: 11. In some embodiments, the first amino acid sequence comprises at least 93% sequence identity to SEQ ID NO: 11. In some embodiments, the first amino acid sequence comprises at least 94% sequence identity to SEQ ID NO: 11. In some embodiments, the first amino acid sequence comprises at least 95% sequence identity to SEQ ID NO: 11. In some embodiments, the first amino acid sequence comprises at least 96% sequence identity to SEQ ID NO: 11. In some embodiments, the first amino acid sequence comprises at least 97% sequence identity to SEQ ID NO: 11. In some embodiments, the first amino acid sequence comprises at least 98% sequence identity to SEQ ID NO: 11. In some embodiments, the first amino acid sequence comprises at least 99% sequence identity to SEQ ID NO: 11.

In some embodiments, a recombinant β-hexosaminidase enzyme disclosed herein comprises 2, 3, 4, 5, 6, 7, 8, 9, 10, 11, 12, 13 14, 15, 16, 17, 18, 19, 20, 21, 22, 23, 24, 25, 26, 27, 28, 2930, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 125, 150, 175, 200, 225, 250,275, 300, 325, 350, 375, 400,425, 450, 475, 500, 525, 550, 575, or 600 contiguous amino acids of any amino acid sequence disclosed herein. In some embodiments, the amino acid sequence is SEQ ID NO: 2. In some embodiments, the amino acid sequence is SEQ ID NO: 3. In some embodiments, the amino acid sequence is SEQ ID NO: 12. In some embodiments, the amino acid sequence is SEQ ID NO: 13.

In some embodiments, the second amino acid sequence is N-terminal to the first amino acid sequence. In some embodiments, the second amino acid sequence comprises at least 20 contiguous amino acid residues of SEQ ID NO: 2. In some embodiments, the second amino acid sequence comprises at least 30 contiguous amino acid residues of SEQ ID NO: 2. In some embodiments, the second amino acid sequence comprises at least 40 contiguous amino acid residues of SEQ ID NO: 2. In some embodiments, the second amino acid sequence comprises at least 50 contiguous amino acid residues of SEQ ID NO: 2. In some embodiments, the second amino acid sequence comprises at least 60 contiguous amino acid residues of SEQ ID NO: 2. In some embodiments, the second amino acid sequence comprises at least 70 contiguous amino acid residues of SEQ ID NO: 2. In some embodiments, the second amino acid sequence comprises at least 80 contiguous amino acid residues of SEQ ID NO: 2. In some embodiments, the second amino acid sequence comprises at least 90 contiguous amino acid residues of SEQ ID NO: 2.

In some embodiments, the second amino acid sequence comprises at least 50 contiguous amino acid residues of SEQ ID NO: 12. In some embodiments, the second amino acid sequence comprises at least 60 contiguous amino acid residues of SEQ ID NO: 12. In some embodiments, the second amino acid sequence comprises at least 70 contiguous amino acid residues of SEQ ID NO: 12. In some embodiments, the second amino acid sequence comprises at least 80 contiguous amino acid residues of SEQ ID NO: 12. In some embodiments, the second amino acid sequence comprises at least 90 contiguous amino acid residues of SEQ ID NO: 12. In some embodiments, the second amino acid sequence comprises at least 100 contiguous amino acid residues of SEQ ID NO: 12.

In some embodiments, the second amino acid sequence comprises at least 50 contiguous amino acid residues of SEQ ID NO: 13. In some embodiments, the second amino acid sequence comprises at least 60 contiguous amino acid residues of SEQ ID NO: 13. In some embodiments, the second amino acid sequence comprises at least 70 contiguous amino acid residues of SEQ ID NO: 13. In some embodiments, the second amino acid sequence comprises at least 80 contiguous amino acid residues of SEQ ID NO: 13. In some embodiments, the second amino acid sequence comprises at least 90 contiguous amino acid residues of SEQ ID NO: 13. In some embodiments, the second amino acid sequence comprises at least 100 contiguous amino acid residues of SEQ ID NO: 13. In some embodiments, the second amino acid sequence comprises at least 150 contiguous amino acid residues of SEQ ID NO: 13. In some embodiments, the second amino acid sequence comprises at least 85% sequence identity to SEQ ID NO: 13. In some embodiments, the second amino acid sequence comprises at least 90% sequence identity to SEQ ID NO: 13. In some embodiments, the second amino acid sequence comprises at least 95% sequence identity to SEQ ID NO: 13. In some embodiments, the second amino acid sequence comprises the amino acid sequence of SEQ ID NO: 13.

In some embodiments, the β-hexosaminidase variant α subunit comprises an amino acid sequence with at least 70% sequence identity to SEQ ID NO: 3. In some embodiments, the β-hexosaminidase variant α subunit comprises an amino acid sequence with at least 75% sequence identity to SEQ ID NO: 3. In some embodiments, the β-hexosaminidase variant α subunit comprises an amino acid sequence with at least 80% sequence identity to SEQ ID NO: 3. In some embodiments, the β-hexosaminidase variant α subunit comprises an amino acid sequence with at least 85% sequence identity to SEQ ID NO: 3. In some embodiments, the β-hexosaminidase variant α subunit comprises an amino acid sequence with at least 90% sequence identity to SEQ ID NO: 3. In some embodiments, the β-hexosaminidase variant α subunit comprises an amino acid sequence with at least 95% sequence identity to SEQ ID NO: 3. In some embodiments, the β-hexosaminidase variant α subunit comprises an amino acid sequence with at least 99% sequence identity to SEQ ID NO: 3. In some embodiments, the β-hexosaminidase variant α subunit comprises the amino acid sequence according to SEQ ID NO: 3. In some embodiments, the β-hexosaminidase variant α subunit comprises at least 500 contiguous amino acids of an amino acid sequence according to SEQ ID NO: 3. In some embodiments, the β-hexosaminidase variant α subunit comprises at least 525 contiguous amino acids of an amino acid sequence according to SEQ ID NO: 3. In some embodiments, the β-hexosaminidase variant α subunit comprises at least 550 contiguous amino acids of an amino acid sequence according to SEQ ID NO: 3. In some embodiments, the β-hexosaminidase variant α subunit comprises at least 500 contiguous amino acids of an amino acid sequence according to SEQ ID NO: 3, and at least 95% sequence identity to the at least 500 contiguous amino acids. In some embodiments, the β-hexosaminidase variant α subunit comprises at least 525 contiguous amino acids of an amino acid sequence according to SEQ ID NO: 3, and at least 95% sequence identity to the at least 525 contiguous amino acids. In some embodiments, the β-hexosaminidase variant α subunit comprises at least 550 contiguous amino acids of an amino acid sequence according to SEQ ID NO: 3, and at least 95% sequence identity to the at least 550 contiguous amino acids.

In some embodiments, a recombinant β-hexosaminidase variant α subunit disclosed herein is truncated by 1, 2, 3, 4, 5, 6, 7, 8, 9, 10, 11, 12, 13 14, 15, 16, 17, 18, 19, 20, 21, 22, 23, 24, 25, 26, 27, 28, 29 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, or 100 amino acids relative to an amino acid sequence disclosed herein.

As used herein, the abbreviations for the L-enantiomeric and D-enantiomeric amino acids are as follows: alanine (A, Ala); arginine (R, Arg); asparagine (N, Asn); aspartic acid (D, Asp); cysteine (C, Cys); glutamic acid (E, Glu); glutamine (Q, Gln); glycine (G, Gly); histidine (H, His); isoleucine (I, Ile); leucine (L, Leu); lysine (K, Lys); methionine (M, Met); phenylalanine (F, Phe); proline (P, Pro); serine (S, Ser); threonine (T, Thr); tryptophan (W, Trp); tyrosine (Y, Tyr); valine (V, Val). In some embodiments, the amino acid is a L-enantiomer. In some embodiments, the amino acid is a D-enantiomer.

A β-hexosaminidase variant α subunit homodimer disclosed herein can comprise a sequence as disclosed in TABLE 2 below.

TABLE 2
Sequences for ß-hexosaminidase variant a subunit homodimer disclosed herein

| | Construct | | SEQ ID |
|---|---|---|---|
| Construct Name | Description | Sequence | NO: |
| Hex A alpha | Wild type | MTSSRLWFSLLLAAAFAGRATALWPWPQNFQTSDQRY | 1 |
| subunit (protein | beta- | VLYPNNFQFQYDVSSAAQPGCSVLDEAFQRYRDLLFGS | |
| sequence) | hexo | GSWPRPYLTGKRHTLEKNVLVVSVVTPGCNQLPTLESV | |
| | saminidase | ENYTLTINDDQCLLLSETVWGALRGLETFSQLVWKSAE | |
| | alpha | GTFFINKTEIEDFPRFPHRGLLLDTSRHYLPLSSILDTLDV | |
| | subunit | MAYNKLNVFHWHLVDDPSFPYESFTFPELMRKGSYNPV | |
| | (Theoretical | THIYTAQDVKEVIEYARLRGIRVLAEFDTPGHTLSWGPGI | |
| | pI/Mw: | PGLLTPCYSGSEPSGTFGPVNPSLNNTYEFMSTFFLEVSS | |
| | 4.95/ | VFPDFYLHLGGDEVDFTCWKSNPEIQDFMRKKGFGEDF | |
| | 58379.09) | KQLESFYIQTLLDIVSSYGKGYVVWQEVFDNKVKIQPDT | |
| | | IIQVWREDIPVNYMKELELVTKAGFRALLSAPWYLNRIS | |
| | | YGPDWKDFYIVEPLAFEGTPEQKALVIGGEACMWGEYV | |
| | | DNTNLVPRLWPRAGAVAERLWSNKLTSDLTFAYERLSH | |
| | | FRCELLRRGVQAQPLNVGFCEQEFEQT | |
| Hex A beta | Wild type | MELCGLGLPRPPMLLALLLATLLAAMLALLTQVALVVQ | 2 |
| subunit (protein | beta- | VAEAARAPSVSAKPGPALWPLPLSVKMTPNLLHLAPEN | |
| sequence) | hexo- | FYISHSPNSTAGPSCTLLEEAFRYHGYIFGFYKWHHEPAE | |
| | saminidase | FQAKTQVQQLLVSITLQSECDAFPNISSDESYTLLVKEPV | |
| | beta | AVLKANRVWGALRGLETFSQLVYQDSYGTFTINESTIID | |
| | subunit | SPRFSHRGILIDTSRHYLPVKIILKTLDAMAFNKFNVLHW | |
| | (Theoretical | HIVDDQSFPYQSITFPELSNKGSYSLSHVYTPNDVRMVIE | |
| | pI/Mw: | YARLRGIRVLPEFDTPGHTLSWGKGQKDLLTPCYSRQN | |
| | 6.43/ | KLDSFGPINPTLNTTYSFLTTFFKEISEVFPDQFIHLGGDE | |
| | 58786.88) | VEFKCWESNPKIQDFMRQKGFGTDFKKLESFYIQKVLDI | |
| | | IATINKGSIVWQEVFDDKAKLAPGTIVEVWKDSAYPEEL | |
| | | SRVTASGFPVILSAPWYLDLISYGQDWRKYYKVEPLDFG | |
| | | GTQKQKQLFIGGEACLWGEYVDATNLTPRLWPRASAV | |
| | | GERLWSSKDVRDMDDAYDRLTRHRCRMVERGIAAQPL | |
| | | YAGYCNHENM | |
| Hex D3 (alpha | Variant | MELCGLGLPRPPMLLALLLATLLAAMLALLTQVALVVQ | 3 |
| subunit) (protein | beta- | VAEAARAPSVSAKPGPALWPLPLSVKMTPNLLHLAPEN | |
| sequence) | hexo- | FYISHSPNSTAGPSCTLLEEAFRRYHGYIFGFYKWHHEPA | |
| | saminidase | EFQAKTQVQQLLVSITLQSECDAFPNISSDESYTLLVKEP | |
| | alpha subunit | VAVLKANRVWGALRGLETFSQLVYQDSYGTFTINESTII | |
| | | DSPRFPHRGLLLDTSRHYLPLKSILDTLDVMAYNKLNVF | |
| | | HWHLVDDQSFPYESFTFPELMRKGSYSLSHIYTAQDVKE | |
| | | VIEYARLRGIRVLAEFDTPGHTLSWGPGIPGLLTPCYSGS | |
| | | EPSGTFGPVNPSLNNTYEFMSTFFLEVSSVFPDFYLHLGG | |
| | | DEVDFTCWKSNPEIQDFMRKKGFGEDFKQLESFYIQTLL | |
| | | DIVSSYGKGYVVWQEVFDNKVKIQPDTIIQVWREDIPVN | |
| | | YMKELELVTKAGFRALLSAPWYLNRISYGQDWRKFYK | |
| | | VEPLAFEGTPEQKALVIGGEACMWGEYVDATNLVPRL | |
| | | WPRAGAVAERLWSNKLTRDMDDAYDRLSHFRCELVRR | |
| | | GVAAQPLYAGYCNQEFEQT | |
| Hex A alpha | Wild type | ATGACTTCCTCCCGCCTTTGGTTCTCCCTCCTGCTTGC | 4 |
| subunit | beta- | CGCTGCCTTCGCCGGACGCGCCACCGCCCTGTGGCCG | |
| (nucleotide | hexo- | TGGCCTCAGAACTTCCAGACTAGCGACCAAAGATACG | |
| sequence) | saminidase | TGCTGTACCCGAACAACTTCCAGTTTCAATACGACGT | |
| | alpha subunit | CAGCAGCGCCGCCCAGCCCGGCTGCTCGGTGCTCGAT | |
| | | GAGGCATTCCAGCGGTACCGGGATCTCTTGTTCGGTT | |
| | | CCGGATCATGGCCTCGGCCGTACCTCACTGGAAAGAG | |
| | | GCACACTCTCGAAAAGAACGTCCTGGTGGTGTCCGTG | |
| | | GTCACCCCTGGCTGCAATCAGCTGCCCACCCTGGAAT | |
| | | CAGTGGAGAACTACACTITGACCATCAACGATGACCA | |
| | | ATGCCTGCTGCTGTCGGAGACTGTGTGGGGTGCCCTG | |
| | | CGCGGGCTGGAAACCTTTAGCCAACTGGTCTGGAAGT | |
| | | CAGCCGAGGGGACCTTCTTCATTAACAAGACCGAAAT | |
| | | CGAGGACTTCCCTCGGTTCCCGCACCGCGGCTTGCTG | |
| | | CTGGATACCTCGCGGCACTATCTGCCACTGTCCTCCAT | |
| | | TCTCGATACCCTGGACGTGATGGCCTACAACAAGCTG | |
| | | AACGTGTTCCACTGGCATCTCGTGGACGACCCATCCT | |
| | | TTCCCTACGAGTCCTTCACCTTCCCTGAGTTGATGAGA | |
| | | AAGGGCTCCTACAACCCCGTGACCCATATCTACACGG | |
| | | CTCAGGACGTGAAGGAAGTGATCGAATATGCCCGGCT | |
| | | GAGAGGGATTAGGGTGCTGGCAGAATTTGACACCCCG | |
| | | GGACACACCCTGTCGTGGGGCCCTGGTATCCCGGGCC | |
| | | TGCTGACTCCGTGCTACTCCGGCTCCGAGCCATCCGG | |
| | | AACCTTCGGACCTGTGAATCCCTCCCTGAACAACACT | |
| | | TACGAGTTCATGTCCACCTTCTTCCTGGAAGTGTCGAG | |
| | | CGTGTTCCCCGACTTCTACCTCCACCTCGGTGGCGACG | |
| | | AAGTCGATTTCACTTGCTGGAAGTCTAACCCCGAGAT | |
| | | CCAAGATITCATGCGAAAGAAAGGATTCGGAGAGGA | |
| | | CTTTAAGCAGCTGGAGTCCTTCTACATCCAAACCCTG | |
| | | CTGGACATTGTGTCATCGTATGGAAAGGGATACGTGG | |
| | | TGTGGCAGGAAGTGTTTGACAATAAGGTCAAAATTCA | |
| | | GCCCGATACAATCATCCAAGTCTGGCGCGAAGATATC | |
| | | CCCGTGAACTACATGAAAGAACTGGAACTGGTCACGA | |
| | | AGGCTGGATTCAGGGCGCTTCTGAGCGCCCCTTGGTA | |
| | | CTTGAACCGGATTAGCTACGGCCCGGACTGGAAGGAC | |
| | | TTCTACATCGTCGAACCTCTGGCCTTCGAGGGAACCC | |
| | | CCGAGCAGAAGGCCTTGGTGATCGGCGGCGAAGCCT | |
| | | GTATGTGGGGGGAATACGTGGACAACACCAACCTGGT | |
| | | GCCGCGCCTGTGGCCGAGAGCGGGAGCAGTGGCCGA | |
| | | GCGGCTCTGGTCGAACAAGCTGACTTCCGACCTCACC | |
| | | TTCGCATACGAGAGACTGAGCCACTTCCGCTGCGAAC | |
| | | TTCTTCGGCGGGGGGTGCAGGCCCAGCCGCTCAACGT | |
| | | CGGGTTCTGTGAACAGGAGTTCGAGCAGACC | |
| | Wild type | ATGGAGCTGTGCGGGCTGGGGCTGCCCCGGCCGCCCA | 5 |
| Hex A beta | beta- | TGCTGCTGGCGCTGCTGTTGGCGACACTGCTGGCGGC | |
| subunit | hexo | GATGTTGGCGCTGCTGACTCAGGTGGCGCTGGTGGTG | |
| (nucleotide | saminidase | CAGGTGGCGGAGGCGGCTCGGGCCCCGAGCGTCTCG | |
| sequence) | beta | GCCAAGCCGGGGCCGGCGCTGTGGCCCCTGCCGCTCT | |
| | subunit | TGGTGAAGATGACCCCGAACCTGCTGCATCTCGCCCC | |
| | | GGAGAACTTCTACATCAGCCACAGCCCCAATTCCACG | |
| | | GCGGGCCCCTCCTGCACCCTGCTGGAGGAAGCGTTTC | |
| | | GACGATATCATGGCTATATTTTTGGTTTCTACAAGTGG | |
| | | CATCATGAACCTGCTGAATTCCAGGCTAAAACCCAGG | |
| | | TTCAGCAACTTCTTGTCTCAATCACCCTTCAGTCAGAG | |
| | | TGTGATGCTTTCCCCAACATATCTTCAGATGAGTCTTA | |
| | | TACTTTACTTGTGAAAGAACCAGTGGCTGTCCTTAAG | |
| | | GCCAACAGAGTTTGGGGAGCATTACGAGGTITAGAGA | |
| | | CCTTTAGCCAGTTAGTTTATCAAGATTCTTATGGAACT | |
| | | TTCACCATCAATGAATCCACCATTATTGATTCTCCAAG | |
| | | GTTTTCTCACAGAGGAATTTTGATTGATACATCCAGAC | |
| | | ATTATCTGCCAGTTAAGATTATTCTTAAAACTCTGGAT | |
| | | GCCATGGCTTTTAATAAGTTTAATGTTCTTCACTGGCA | |
| | | CATAGTIGATGACCAGTCTTTCCCATATCAGAGCATC | |
| | | ACTTTTCCTGAGTTAAGCAATAAAGGAAGCTATTCTTT | |
| | | GTCTCATGTTTATACACCAAATGATGTCCGTATGGTGA | |
| | | TTGAATATGCCAGATTACGAGGAATTCGAGTCCTGCC | |
| | | AGAATITGATACCCCTGGGCATACACTATCTTGGGGA | |
| | | AAAGGTCAGAAAGACCTCCTGACTCCATGTTACAGTA | |
| | | GACAAAACAAGTTGGACTCTTTTGGACCTATAAACCC | |
| | | TACTCTGAATACAACATACGCTTCCTTACTACATTTTT | |
| | | CAAAGAAATTAGTGAGGTGTTTCCAGATCAATTCATT | |
| | | CATTTGGGAGGAGATGAAGTGGAATTTAAATGTTGGG | |
| | | AATCAAATCCAAAAATTCAAGATTTCATGAGGCAAAA | |
| | | AGGCTTTGGCACAGATTTTAAGAAACTAGAATCTTTC | |
| | | TACATTCAAAAGGTTTTGGATATTATTGCAACCATAA | |
| | | ACAAGGGATCCATTGTCTGGCAGGAGGTTTTTGATGA | |
| | | TAAAGCAAAGCTTGCGCCGGGCACAATAGTTGAAGTA | |
| | | TGGAAAGACAGCGCATATCCTGAGGAACTCAGTAGA | |
| | | GTCACAGCATCTGGCTTCCCTGTAATCCTTTCTGCTCC | |
| | | TTGGTACTTAGATTTGATTAGCTATGGACAAGATTGG | |
| | | AGGAAATACTATAAAGTGGAACCTCTTGATTTTGGCG | |
| | | GTACTCAGAAACAGAAACAACTTTTCATTGGTGGAGA | |
| | | AGCTTGTCTATGGGGAGAATATGTGGATGCAACTAAC | |
| | | CTCACTCCAAGATTATGGCCTCGGGCAAGTGCTGTTG | |
| | | GTGAGAGACTCTGGAGTTCCAAAGATGTCAGAGATAT | |
| | | GGATGACGCCTATGACAGACTGACAAGGCACCGCTGC | |
| | | AGGATGGTCGAACGTGGAATAGCTGCACAACCTCTTT | |
| | | ATGCTGGATATTGTAACCATGAGAACATGTAA | |
| Hex M (alpha | Variant | MTSSRLWFSLLLAAAFAGRATALWPWPQNFQTSDQRY | 6 |
| subunit) (protein | beta- | VLYPNNFQFQYDVSSAAQPGCSVLDEAFQRYRDLLFGS | |
| sequence) | hexo- | GSWPRPYLTGKRHTLEKNVLVVSVVTPGCNQLPTLESV | |
| | saminidase | ENYTLTINDDQCLLLSETVWGALRGLETFSQLVWKSAE | |
| | alpha subunit | GTFFINKTEIEDFPRFPHRGLLLDTSRHYLPLKSILDTLDV | |
| | | MAYNKLNVFHWHLVDDQSFPYESFTFPELMRKGSYSLS | |
| | | HIYTAQDVKEVIEYARLRGIRVLAEFDTPGHTLSWGPGIP | |
| | | GLLTPCYSGSEPSGTFGPVNPSLNNTYEFMSTFFLEVSSV | |
| | | FPDFYLHLGGDEVDFTCWKSNPEIQDFMRKKGFGEDFK | |
| | | QLESFYIQTLLDIVSSYGKGYVVWQEVFDNKVKIQPDTII | |
| | | QVWREDIPVNYMKELELVTKAGFRALLSAPWYLNRISY | |
| | | GQDWRKFYKVEPLAFEGTPEQKALVIGGEACMWGEYV | |
| | | DATNLVPRLWPRAGAVAERLWSNKLTRDMDDAYDRLS | |
| | | HFRCELVRRGVAAQPLYAGYCNQEFEQT | |
| Hex M (alpha | Variant | ATGACTTCCTCCCGCCTTTGGTTCTCCCTCCTGCTTGC | 7 |
| subunit) | beta- | CGCTGCCTTCGCCGGACGCGCCACCGCCCTGTGGCCG | |
| (nucleotide | hexo- | TGGCCTCAGAACTTCCAGACTAGCGACCAAAGATACG | |
| sequence) | saminidase | TGCTGTACCCGAACAACTTCCAGTTTCAATACGACGT | |
| | alpha subunit | CAGCAGCGCCGCCCAGCCCGGCTGCTCGGTGCTCGAT | |
| | | GAGGCATTCCAGCGGTACCGGGATCTCTTGTTCGGTT | |
| | | CCGGATCATGGCCTCGGCCGTACCTCACTGGAAAGAG | |
| | | GCACACTCTCGAAAAGAACGTCCTGGTGGTGTCCGTG | |
| | | GTCACCCCTGGCTGCAATCAGCTGCCCACCCTGGAAT | |
| | | CAGTGGAGAACTACACTTTGACCATCAACGATGACCA | |
| | | ATGCCTGCTGCTGTCGGAGACTGTGTGGGGTGCCCTG | |
| | | CGCGGGCTGGAAACCTTTAGCCAACTGGTCTGGAAGT | |
| | | CAGCCGAGGGGACCTTCTTCATTAACAAGACCGAAAT | |
| | | CGAGGACTTCCCTCGGTTCCCGCACCGCGGCTTGCTG | |
| | | CTGGATACCTCGCGGCACTATCTGCCACTGAAGTCCA | |
| | | TTCTCGATACCCTGGACGTGATGGCCTACAACAAGCT | |
| | | GAACGTGTTCCACTGGCATCTCGTGGACGACCAGTCC | |
| | | TTTCCCTACGAGTCCTTCACCTTCCCTGAGTTGATGAG | |
| | | AAAGGGCTCCTACTCCCTCTCCCATATCTACACGGCTC | |
| | | AGGACGTGAAGGAAGTGATCGAATATGCCCGGCTGA | |
| | | GAGGGATTAGGGTGCTGGCAGAATTTGACACCCCGGG | |
| | | ACACACCCTGTCGTGGGGCCCTGGTATCCCGGGCCTG | |
| | | CTGACTCCGTGCTACTCCGGCTCCGAGCCATCCGGAA | |
| | | CCTTCGGACCTGTGAATCCCTCCCTGAACAACACTTA | |
| | | CGAGTTCATGTCCACCTTCTTCCTGGAAGTGTCGAGC | |
| | | GTGTTCCCCGACTTCTACCTCCACCTCGGTGGCGACG | |
| | | AAGTCGATTTCACTTGCTGGAAGTCTAACCCCGAGAT | |
| | | CCAAGATITCATGCGAAAGAAAGGATTCGGAGAGGA | |
| | | CTTTAAGCAGCTGGAGTCCTTCTACATCCAAACCCTG | |
| | | CTGGACATTGTGTCATCGTATGGAAAGGGATACGTGG | |
| | | TGTGGCAGGAAGTGTTTGACAATAAGGTCAAAATTCA | |
| | | GCCCGATACAATCATCCAAGTCTGGCGCGAAGATATC | |
| | | CCCGTGAACTACATGAAAGAACTGGAACTGGTCACGA | |
| | | AGGCTGGATTCAGGGCGCTTCTGAGCGCCCCTTGGTA | |
| | | CTTGAACCGGATTAGCTACGGCCAGGACTGGAGGAA | |
| | | GTTCTACAAGGTCGAACCTCTGGCCTTCGAGGGAACC | |
| | | CCCGAGCAGAAGGCCTTGGTGATCGGCGGCGAAGCCT | |
| | | GTATGTGGGGGGAATACGTGGACGCGACCAACCTGGT | |
| | | GCCGCGCCTGTGGCCGAGAGCGGGAGCAGTGGCCGA | |
| | | GCGGCTCTGGTCGAACAAGCTGACTAGGGACATGGAC | |
| | | GATGCATACGACAGACTGAGCCACTTCCGCTGCGAAC | |
| | | TTGTGCGGCGGGGGGTGGCGGCCCAGCCGCTCTACGC | |
| | | GGGGTACTGTAACCAGGAGTTCGAGCAGACC | |
| Hex D3 (alpha | Variant | ATGGAACTTTGCGGACTCGGCCTCCCAAGACCACCTA | 8 |
| subunit) | beta- | TGCTTCTCGCCCTGCTGCTCGCCACCTTGCTCGCGGCT | |
| (nucleotide | hexo- | ATGCTTGCGCTCCTGACTCAAGTGGCCCTTGTGGTCCA | |
| sequence) | saminidase | AGTGGCCGAGGCTGCCCGCGCCCCGAGCGTGTCAGCC | |
| | alpha subunit | AAGCCAGGACCGGCCCTGTGGCCGCTGCCTCTGAGCG | |
| | | TGAAGATGACTCCCAATCTCCTGCACCTGGCCCCGGA | |
| | | AAACTTCTACATCTCGCACTCGCCGAACAGCACCGCC | |
| | | GGTCCCTCCTGCACCCTGCTCGAAGAGGCATTCCGGC | |
| | | GGTACCACGGATACATCTTCGGTTTCTATAAGTGGCA | |
| | | TCACGAGCCGGCAGAGTTCCAGGCCAAGACTCAGGTC | |
| | | CAGCAGCTGCTCGTGTCCATTACCCTGCAATCGGAGT | |
| | | GCGACGCCTTCCCCAACATCAGCTCAGACGAGTCATA | |
| | | CACTTTGCTCGTGAAGGAACCTGTCGCCGTGCTGAAG | |
| | | GCCAACCGCGTGTGGGGTGCCCTGCGCGGGCTGGAAA | |
| | | CCTTTAGCCAACTGGTCTACCAAGATTCATACGGGAC | |
| | | CTTCACCATTAACGAGTCCACCATCATCGACTCCCCTC | |
| | | GGTTCCCGCACCGCGGCTTGCTGCTGGATACCTCGCG | |
| | | GCACTATCTGCCACTGAAGTCCATTCTCGATACCCTG | |
| | | GACGTGATGGCCTACAACAAGCTGAACGTGTTCCACT | |
| | | GGCATCTCGTGGACGACCAGTCCTTTCCCTACGAGTC | |
| | | CTTCACCTTCCCTGAGTTGATGAGAAAGGGCTCCTAC | |
| | | TCCCTCTCCCATATCTACACGGCTCAGGACGTGAAGG | |
| | | AAGTGATCGAATATGCCCGGCTGAGAGGGATTAGGGT | |
| | | GCTGGCAGAATTTGACACCCCGGGACACACCCTGTCG | |
| | | TGGGGCCCTGGTATCCCGGGCCTGCTGACTCCGTGCT | |
| | | ACTCCGGCTCCGAGCCATCCGGAACCTTCGGACCTGT | |
| | | GAATCCCTCCCTGAACAACACTTACGAGTTCATGTCC | |
| | | ACCTTCTTCCTGGAAGTGTCGAGCGTGTTCCCCGACTT | |
| | | CTACCTCCACCTCGGTGGCGACGAAGTCGATTTCACT | |
| | | TGCTGGAAGTCTAACCCCGAGATCCAAGATTTCATGC | |
| | | GAAAGAAAGGATTCGGAGAGGACTTTAAGCAGCTGG | |
| | | AGTCCTTCTACATCCAAACCCTGCTGGACATTGTGTCA | |
| | | TCGTATGGAAAGGGATACGTGGTGTGGCAGGAAGTGT | |
| | | TTGACAATAAGGTCAAAATTCAGCCCGATACAATCAT | |
| | | CCAAGTCTGGCGCGAAGATATCCCCGTGAACTACATG | |
| | | AAAGAACTGGAACTGGTCACGAAGGCTGGATTCAGG | |
| | | GCGCTTCTGAGCGCCCCTTGGTACTTGAACCGGATTA | |
| | | GCTACGGCCAGGACTGGAGGAAGTTCTACAAGGTCG | |
| | | AACCTCTGGCCTTCGAGGGAACCCCCGAGCAGAAGGC | |
| | | CTTGGTGATCGGCGGCGAAGCCTGTATGTGGGGGGAA | |
| | | TACGTGGACGCGACCAACCTGGTGCCGCGCCTGTGGC | |
| | | CGAGAGCGGGAGCAGTGGCCGAGCGGCTCTGGTCGA | |
| | | ACAAGCTGACTAGGGACATGGACGATGCATACGACA | |
| | | GACTGAGCCACTTCCGCTGCGAACTTGTGCGGCGGGG | |
| | | GGTGGCGGCCCAGCCGCTCTACGCGGGGTACTGTAAC | |
| | | CAGGAGTTCGAGCAGACC | |
| Mod2B (beta | Variant | ATGGAACTTTGTGGACTTGGACTCCCTCGCCCTCCGAT | 9 |
| subunit) | beta- | GCTGCTTGCACTGCTCCTCGCCACTCTCCTCGCCGCTA | |
| (nucleotide | hexo- | TGCTGGCGCTGCTGACTCAAGTGGCACTGGTGGTCCA | |
| sequence) | saminidase | AGTGGCCGAGGCGGCCCGCGCACCATCCGTGTCCGCG | |
| | beta subunit | AAGCCGGGCCCAGCCCTGTGGCCCCTGCCTCTGAGCG | |
| | | TGAAGATGACCCCTAACCTTCTGCACCTGGCGCCGGA | |
| | | GAACTTCTACATAAGCCACTCGCCCAACTCGACCGCG | |
| | | GGTCCCTCCTGTACCCTGCTGGAGGAGGCCTTCAGGC | |
| | | GCTACCACGGCTACATTTTCGGGTTCTATAAGTGGCA | |
| | | CCACGAGCCGGCCGAGTTCCAAGCCAAGACTCAGGTC | |
| | | CAACAGCTGCTGGTGTCAATCACACTGCAGTCCGAAT | |
| | | GCGATGCTTTCCCTAATATCTCCTCCGACGAGTCCTAC | |
| | | ACCCTGCTCGTGAAGGAACCTGTGGCCGTGCTCAAGG | |
| | | CCAACCGCGTCTGGGGAGCCCTCAGGGGTCTGGAAAC | |
| | | CTTCAGCCAGCTGGTGTACCAGGACAGCTACGGCACC | |
| | | TTCACCATTAACGAGTCCACCATCATCGACTCACCGA | |
| | | GATTCAGCCACCGGGGAATCCTGATTGATACTTCAAG | |
| | | ACACTATTTGCCCGTGAAGATCATCCTGAAAACCCTC | |
| | | GACGCTATGGCCTTTAACAAGTTTAACGTGTTGCATTG | |
| | | GCATATCGTGGACGATCAGTCCTTCCCGTATCAAAGC | |
| | | ATCACTTTTCCGGAACTGTCCAACAAGGGATCTTACT | |
| | | CGCTGAGCCACGTGTACACTCCCAATGACGTGCGCAT | |
| | | GGTCATCGAATACGCCCGGCTGAGGGGCATTAGAGTG | |
| | | CTCCCTGAATTTGACACCCCCGGCCATACCCTCTCCTG | |
| | | GGGAAAAGGACAGAAAGATTTGCTCACACCTTGCTAC | |
| | | TCCGGCTCCGAGCCCTCTGGAACTTTCGGCCCCATCA | |
| | | ACCCGACCCTGAACACCACTTACTCCTTCTTGACCAC | |
| | | GTTCTTCAAGGAGATTTCCGAGGTGTTCCCGGACCAG | |
| | | TTCATCCACCTGGGGGGCGACGAAGTGGAGTTCAAGT | |
| | | GCTGGGAATCGAACCCAAAGATCCAGGACTTCATGAG | |
| | | GCAGAAGGGGTTTGGCACCGATTTCAAGAAGCTGGA | |
| | | GTCGTTCTACATTCAAAAGGTCCTGGACATCATTGCA | |
| | | ACCATCAACAAGGGGTCAATCGTGTGGCAGGAAGTGT | |
| | | TCGATGACAAGGCTAAGCTGGCGCCCGGCACTATTGT | |
| | | GGAAGTCTGGAAAGACTCCGCCTACCCTGAGGAGCTG | |
| | | TCGAGAGTCACCGCCTCCGGATTCCCCGTCATCCTGA | |
| | | GCGCTCCGTGGTACCTGAACCGGATCTCCTACGGACA | |
| | | AGATTGGCGCAAGTACTACAAGGTCGAACCGCTGGAC | |
| | | TTCGGAGGGACTCAGAAGCAGAAGCAGCTCTTTATTG | |
| | | GTGGAGAGGCCTGCCTTTGGGGCGAATACGTGGACGC | |
| | | CACCAACCTCACCCCGCGGCTCTGGCCACGGGCCAGC | |
| | | GCCGTGGGAGAACGCCTGTGGTCGTCAAAGGACGTCC | |
| | | GCGATATGGACGATGCCTACGACCGGCTCACTCGGCA | |
| | | TCGGTGCCGGATGGTGGAACGCGGTATCGCAGCGCAG | |
| | | CCACTGTACGCTGGATACTGCAACCACGAGAATATG | |
| Mod2B (beta | Variant | MELCGLGLPRPPMLLALLLATLLAAMLALLTQVALVVQ | 10 |
| subunit) (protein | beta- | VAEAARAPSVSAKPGPALWPLPLSVKMTPNLLHLAPEN | |
| sequence) | hexo- | FYISHSPNSTAGPSCTLLEEAFRRYHGYIFGFYKWHHEPA | |
| | saminidase | EFQAKTQVQQLLVSITLQSECDAFPNISSDESYTLLVKEP | |
| | beta subunit | VAVLKANRVWGALRGLETFSQLVYQDSYGTFTINESTII | |
| | | DSPRFSHRGILIDTSRHYLPVKIILKTLDAMAFNKFNVLH | |
| | | WHIVDDQSFPYQSITFPELSNKGSYSLSHVYTPNDVRMV | |
| | | IEYARLRGIRVLPEFDTPGHTLSWGKGQKDLLTPCYSGS | |
| | | EPSGTFGPINPTLNTTYSFLTTFFKEISEVFPDQFIHLGGDE | |
| | | VEFKCWESNPKIQDFMRQKGFGTDFKKLESFYIQKVLDI | |
| | | IATINKGSIVWQEVFDDKAKLAPGTIVEVWKDSAYPEEL | |
| | | SRVTASGFPVILSAPWYLNRISYGQDWRKYYKVEPLDF | |
| | | GGTQKQKQLFIGGEACLWGEYVDATNLTPRLWPRASA | |
| | | VGERLWSSKDVRDMDDAYDRLTRHRCRMVERGIAAQP | |
| | | LYAGYCNHENM | |
| HexM fragment | HexM | KSILDTLDVMAYNKLNVFHWHLVDDQSFPYESFTFPEL | 11 |
| | fragment | MRKGSYSLSHIYTAQDVKEVIEYARLRGIRVLAEFDTPG | |
| | | HTLSWGPGIPGLLTPCYSGSEPSGTFGPVNPSLNNTYEFM | |
| | | STFFLEVSSVFPDFYLHLGGDEVDFTCWKSNPEIQDFMR | |
| | | KKGFGEDFKQLESFYIQTLLDIVSSYGKGYVVWQEVFD | |
| | | NKVKIQPDTIIQVWREDIPVNYMKELELVTKAGFRALLS | |
| | | APWYLNRISYGQDWRKFYKVEPLAFEGTPEQKALVIGG | |
| | | EACMWGEYVDATNLVPRLWPRAGAVAERLWSNKLTR | |
| | | DMDDAYDRLSHFRCELVRRGVAAQPLYAGYCN | |
| HexB fragment | | ARAPSVSAKPGPALWPLPLSVKMTPNLLHLAPENFYISH | 12 |
| | | SPNSTAGPSCTLLEEAFRRYHGYIFGFYKWHHEPAEFQA | |
| | | KTQVQQLLVSITLQSECDAFPNISSDESYTLLVKEPVAVL | |
| | | KANRVWGALRGLETFSQLVYQDSYGTFTINESTIIDS | |
| HexB fragment | | MELCGLGLPRPPMLLALLLATLLAAMLALLTQVALVVQ | 13 |
| | | VAEAARAPSVSAKPGPALWPLPLSVKMTPNLLHLAPEN | |
| | | FYISHSPNSTAGPSCTLLEEAFRRYHGYIFGFYKWHHEPA | |
| | | EFQAKTQVQQLLVSITLQSECDAFPNISSDESYTLLVKEP | |
| | | VAVLKANRVWGALRGLETFSQLVYQDSYGTFTINESTII | |
| | | DS | |

### Post-Translational Modifications and Thermal Stability

In some embodiments, the variant hexosaminidase α subunit described herein includes mature forms of the polypeptide. In some embodiments, the β-hexosammnidase variant α subunit includes one or more one or more post-translational modifications, including proteolytic and/or glycolytic processing. A β-hexosaminidase variant α subunit homodimer disclosed herein can comprise a post-translational modification in the form of, for example, glycosylation. The glycosylation can be, for example, N-linked glycosylation, O-linked glycosylation, phosphoserine glycosylation, or C-mannosylation.

In some embodiments, the β-hexosaminidase variant α subunit is glycosylated at selected Asn-X-Ser/Thr (SEQ ID NO: 14) regions. The asparagine residue can further be modified by the N-linked glycosylation of a mannose-6-phosphate (M6P) residue by the N-acetylglucosamine-1-phosphate transferase enzyme.

A β-hexosaminidase variant α subunit disclosed herein can further comprise sites for glycosylation for M6P addition to the a subunit. The addition of M6P can allow for increased cellular uptake of the β-hexosammnidase variant α subunit homodimer, as the cellular uptake is mediated by an uptake by the cation-independent mannose 6-phosphate (M6P) receptor (CI-MIPR). In some embodiments, a β-hexosaminidase variant α subunit homodimer disclosed herein has greater cellular uptake as compared to a wild-type β-hexosammnidase enzyme or, for example, HexM. A β-hexosaminidase variant α subunit homodimer disclosed herein can display a cellular uptake (Kᵤₚₜₐₖₑ) of, for example, about 500 nM, about 300 nM, about 200 nM, about 100 nM, about 90 nM, about 80 nM, about 70 nM, about 60 nM, about 50 nM, about 40 nM, about 30 nM, about 20 nM, about 10 nM, about 5 nM, about 4 nM, about 3 nM, about 2 nM, or about 1 nM. In some embodiments, a variant β-hexosaminidase enzyme disclosed herein has a Kᵤₚₜₐₖₑ of 3 nM to 13 nM.

In some embodiments, the β-hexosaminidase variant α subunit homodimer comprises an increased mannose-6-phosphorylation (M6P) relative to a homodimer of SEQ ID NO: 6. In some embodiments, the number of M6P sites occupied by M6P per homodimer is increased relative to a homodimer of SEQ ID NO: 6. In some embodiments, the number of M6P sites occupied by M6P per homodimer is 2, 3, 4, 5, 6, 7, 8, 9, or 10. In some embodiments, the number of M6P sites occupied by M6P per homodimer is 3. In some embodiments, the number of M6P sites occupied by M6P per homodimer is 4.

In some embodiments, the β-hexosaminidase variant α subunit homodimer has increased thermal stability relative to a heterodimer of SEQ ID NO: 1 and SEQ ID NO: 2. In some embodiments, the β-hexosaminidase variant α subunit homodimer has increased thermal stability relative to a homodimer of SEQ ID NO: 1. In some embodiments, the β-hexosaminidase variant α subunit homodimer has a melting point (Tm) of about 58° C. to about 63° C. In some embodiments, the β-hexosaminidase variant α subunit homodimer has a melting point (Tm) of 58° C. to 63° C. In some embodiments, the β-hexosaminidase variant α subunit homodimer has a melting point (Tm) of 59° C. to 62° C. In some embodiments, the β-hexosaminidase variant α subunit homodimer has a melting point (Tm) of 59° C. to 62° C. In some embodiments, the β-hexosaminidase variant α subunit homodimer has a melting point (Tm) of 59° C. to 61° C. In some embodiments, the β-hexosaminidase variant α subunit homodimer has a melting point (Tm) of 59.4° C. to 60.2° C. In some embodiments, the β-hexosaminidase variant α subunit homodimer has a melting point (Tm) of 59° C. In some embodiments, the β-hexosaminidase variant α subunit homodimer has a melting point (Tm) of 60° C. In some embodiments, the β-hexosaminidase variant α subunit homodimer has a melting point (Tm) of 59.8° C. In some embodiments, the melting point is measured using differential scanning calorimetry.

### Improved Properties for Use in Treating TSD and SD

In some embodiments, the β-hexosaminidase variant α subunit homodimer exhibits GM2 ganglioside hydrolysis activity in the presence of GM2-activator protein. In some embodiments, the cellular uptake of the β-hexosaminidase variant α subunit homodimer is increased by at least 2-fold, 5-fold, 10-fold, 20-fold, or 25-fold relative to a homodimer of SEQ ID NO: 6. In some embodiments, the cellular uptake of the variant the β-hexosaminidase variant α subunit homodimer is assessed by uptake of cation independent mannose-6-phosphate receptor (CI-MPR) in a competition assay of the homodimer and mannose-6-phosphate in human fibroblasts from SD patients.

In some embodiments, the β-hexosaminidase variant α subunit homodimer increases lifespan of a Hexb knockout mouse when administered at least age 80 days of age as compared to a heterodimer of SEQ ID NO: 1 and SEQ ID NO: 2 that is administered to a Hexb knockout mouse under substantially equivalent assay conditions and age.

In some embodiments, the β-hexosaminidase variant α subunit homodimer increases lifespan by at least 2-fold of a Hexb knockout mouse when administered at least 80 days of age as compared to a heterodimer of SEQ ID NO: 1 and SEQ ID NO: 2 that is administered to a Hexb knockout mouse under substantially equivalent assay conditions and age.

In some embodiments, models for use in testing the improved properties of the β-hexosaminidase variant α subunit homodimers relative to HexA or HexM include mouse models.

SD (HexB gene knockout; loss of β-subunit) mice are deficient in HexA, accumulate ganglioside GM2, and show a severe neurodegenerative disease progression culminating in lower limb spasticity, motor deficit and a shortened lifespan.

TSD (HexA gene deficient; loss of a subunit) mice are also deficient in HexA but bypass the requirement of HexA by the action of a murine sialidase, which converts GM2 to the glycolipid GA2. GA2 can then be metabolized by HexB. In a GM2AP deficient mouse, GA2 can still be metabolized by the low activity of murine HexA and HexB enzymes in absence of GM2AP, resulting in a weaker phenotype.

SD feline models can also be used to study LSDs. SD feline models have a mutation that causes a premature stop codon in the HEXB gene. These felines have less than 3% HexA and HexB activity which is similar to enzyme activity in affected humans with SD. SD feline models have phenotypic and biochemical similarities to human patients. The SD feline models accumulate gangliosides similar to that of humans, locally and abundantly. The SD feline animal models develop normally up to four to seven weeks, but then begin to develop ataxia, seizures, and overall muscle weakness.

The TSD sheep model contain a missense mutation that affects the HEXA gene, which leads the sheep model to produce about 29% functional HexA enzyme activity, similar to that of affected human pathology. The deficiency in HexA leads to subsequent neuropathology through the central and peripheral nervous system that is similar to that seen in human TSD patients.

A biomarker can be used to assess the activity, efficacy, and therapeutic effect of any β-hexosaminidase variant α subunit or β-hexosaminidase variant α subunit homodimer disclosed herein. A biomarker that can be measured in a method disclosed herein includes, for example, gangliosides, GM2 ((2S,3R,4E)-3-Hydroxy-2-(octadecanoylamino)octadec-4-en-1-yl 2-acetamido-2-deoxy-β-D-galactopyranosyl-(1→4)-[5-acetamido-3,5-dideoxy-D-glycero-α-D-galacto-non-2-ulopyranonosyl-(2→3)]-β-D-galactopyranosyl-(1→4)-β-D-glucopyranoside that is associated with TSD and is typically hydrolyzed to GM3 ganglioside in the lysosomes of healthy subjects), GA2 (a representative N-glycan substrate A2G0′ containing beta-linked terminal N-acetyl-D-hexosamine), BMP(22:6) phospholipid (Bis[monoacylglycero]phosphate)), neurofilament light chain (NF-L), or a free glycan.

BMP phospholipid 22:6 (BMP 22:6) is responsible for the degradation, recycling, and chaperoning of molecules, such as cholesterol, in and out of the lysosome. Upon lysosomal stress due to over accumulation of incompletely degraded metabolic products, BMP phospholipids can increase with the most abundant species being BMP 22:6 (two acyl chains each with 22 carbons and six double bonds). Thus, the presence of BMP 22:6 can be an indicator for the presence or progression of SD.

Free glycans are products from the degradation of glycoproteins. HexB plays an important role in the degradation of both N- and O-linked glycans found in glycoproteins by cleaving N-acetylhexosamine at the non-reducing end of these structures. SD patients who are deficient in HexA and B enzymes display a more severe phenotype than TSD patients because they accumulate soluble glycans alongside accumulating gangliosides.

Any β-hexosaminidase variant α subunit or β-hexosaminidase variant α subunit homodimer disclosed herein can be tested in various cell lines. A cell line used herein can include, for example, human embryonic kidney (HEK) 293, Chinese hamster ovary (CHO), monkey kidney (COS), HT1080, C10, HeLa, baby hamster kidney (BHK), 3T3, C127, CV-1, HaK, NS/0, and L-929 cells. Non-limiting examples of mammalian cells that can be used to test any fusion protein disclosed herein include BALB/c mouse myeloma line (NS0/1, ECACC No: 85110503); human retinoblasts (PER.C6); monkey kidney CV1 line transformed by SV40 (COS-7, ATCC CRL 1651); baby hamster kidney cells (BHK, ATCC CCL 10); Chinese hamster ovary cells+/−DHFR (CHO); mouse sertoli cells (TM4); monkey kidney cells (CV1 ATCC CCL 70); African green monkey kidney cells (VERO-76, ATCC CRL-1 587); human cervical carcinoma cells (HeLa, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells; MRC 5 cells; FS4 cells; and a human hepatoma line (Hep G2). In some embodiments, a fusion protein disclosed herein is produced from CHO cell lines.

Any β-hexosaminidase variant α subunit or β-hexosaminidase variant α subunit homodimer disclosed herein can also be expressed in a variety of non-mammalian host cells such as, for example, insect (e.g., Sf-9, Sf-21, Hi5), plant (e.g., Leguminosa, cereal, or tobacco), yeast (e.g., S. cerivisae, P. pastoris), prokaryote (e.g., E. Coli, B. subtilis and other Bacillus spp., Pseudomonas spp., Streptomyces spp), or fungus.

Percent (%) sequence identity with respect to a reference polypeptide sequence is the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are known for instance, using publicly available computer software such as BLAST, BLAST-2, ALIGN or Megalign (DNASTAR) software. Appropriate parameters for aligning sequences are able to be determined, including algorithms needed to achieve maximal alignment over the full length of the sequences being compared. For purposes herein, however, % amino acid sequence identity values are generated using the sequence comparison computer program ALIGN-2 unless stated otherwise. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif, or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program

### β-Hexosaminidase Variant α Subunit Homodimers

Disclosed herein are β-hexosaminidase variant α subunit homodimers comprising a recombinant β-hexosaminidase variant α subunit that comprises one or more amino acid sequence substitutions or deletions at positions corresponding to S184, P209, N228, P229, V230, T231, P429, K432, D433, 1436, N466, 5491, L493, T494, F495, E498, L508, Q513, N518, V519, F521, and E523 of native β-hexosaminidase α subunit of SEQ ID NO:1 and further comprising one or more amino acid sequence elements that increase cellular uptake of the β-hexosaminidase variant α subunit homodimer relative to a homodimer of SEQ ID NO: 6. In some embodiments, the one or more amino acid sequence elements that increase cellular uptake are selected from the group consisting of amino acid sequence substitutions, additions, or deletions relative to the native β-hexosaminidase α subunit of SEQ ID NO:1. In some embodiments, the β-hexosaminidase variant α subunit comprises one or more amino acid substitutions or deletions corresponding to S184K, P209Q, N228S, P229Δ, V230L, T231S, P429Q, K432R, D433K, 1436K, N466A, S491R, L493M, T494D, F495D, E498D, L508V, Q513V, N518Y, V519A, F521Y, and E523N relative to the native β-hexosaminidase α subunit of SEQ ID NO:1. In some embodiments, the β-hexosaminidase variant α subunit comprises at least five amino acid substitutions or deletions corresponding to S184K, P209Q, N228S, P229Δ, V230L, T231S, P429Q, K432R, D433K, 1436K, N466A, S491R, L493M, T494D, F495D, E498D, L508V, Q513V, N518Y, V519A, F521Y, and E523N relative to the native β-hexosaminidase α subunit of SEQ ID NO:1. In some embodiments, the β-hexosaminidase variant α subunit comprises at least ten amino acid substitutions or deletions corresponding to S184K, P209Q, N228S, P229Δ, V230L, T231S, P429Q, K432R, D433K, 1436K, N466A, S491R, L493M, T494D, F495D, E498D, L508V, Q513V, N518Y, V519A, F521Y, and E523N relative to the native β-hexosaminidase α subunit of SEQ ID NO:1. In some embodiments, the β-hexosaminidase variant α subunit comprises at least fifteen amino acid substitutions or deletions corresponding to S184K, P209Q, N228S, P229Δ, V230L, T231S, P429Q, K432R, D433K, 1436K, N466A, S491R, L493M, T494D, F495D, E498D, L508V, Q513V, N518Y, V519A, F521Y, and E523N relative to the native β-hexosaminidase α subunit of SEQ ID NO:1. In some embodiments, the β-hexosaminidase variant α subunit comprises at least twenty amino acid substitutions or deletions corresponding to S184K, P209Q, N228S, P229Δ, V230L, T231S, P429Q, K432R, D433K, 1436K, N466A, S491R, L493M, T494D, F495D, E498D, L508V, Q513V, N518Y, V519A, F521Y, and E523N relative to the native β-hexosaminidase α subunit of SEQ ID NO:1. In some embodiments, the β-hexosaminidase variant α subunit comprises amino acid substitutions or deletions corresponding to S184K, P209Q, N228S, P229Δ, V230L, T231S, P429Q, K432R, D433K, 1436K, N466A, S491R, L493M, T494D, F495D, E498D, L508V, Q513V, N518Y, V519A, F521Y, and E523N relative to the native β-hexosaminidase α subunit of SEQ ID NO: 1. In some embodiments, the β-hexosaminidase variant α subunit comprises a first amino acid sequence comprising at least 85% sequence identity to SEQ ID NO: 11 and the one or more amino acid sequence elements that increase cellular uptake of the β-hexosaminidase variant α subunit relative to a homodimer of SEQ ID NO: 6 comprises a second amino acid sequence. In some embodiments, the first amino acid sequence comprises at least 90% sequence identity to SEQ ID NO: 11. In some embodiments, the first amino acid sequence comprises at least 95% sequence identity to SEQ ID NO: 11. In some embodiments, the first amino acid sequence comprises at least 99% sequence identity to SEQ ID NO: 11. In some embodiments, the first amino acid sequence comprises the amino acid sequence of SEQ ID NO: 11. In some embodiments, the second amino acid sequence is N-terminal to the first amino acid sequence. In some embodiments, the second amino acid sequence comprises at least 20 contiguous amino acid residues of SEQ ID NO: 2. In some embodiments, the second amino acid sequence comprises at least 100 contiguous amino acid residues of SEQ ID NO: 12. In some embodiments, the second amino acid sequence comprises at least 100 contiguous amino acid residues of SEQ ID NO: 13. In some embodiments, the second amino acid sequence comprises at least 150 contiguous amino acid residues of SEQ ID NO: 13. In some embodiments, the second amino acid sequence comprises at least 85% sequence identity to SEQ ID NO: 13. In some embodiments, the second amino acid sequence comprises at least 90% sequence identity to SEQ ID NO: 13. In some embodiments, the second amino acid sequence comprises at least 95% sequence identity to SEQ ID NO: 13. In some embodiments, the second amino acid sequence comprises the amino acid sequence of SEQ ID NO: 13. In some embodiments, the β-hexosaminidase variant α subunit comprises an amino acid sequence with at least 90% sequence identity to SEQ ID NO: 3. In some embodiments, the β-hexosaminidase variant α subunit comprises an amino acid sequence with at least 95% sequence identity to SEQ ID NO: 3. In some embodiments, the β-hexosaminidase variant α subunit comprises an amino acid sequence with at least 99% sequence identity to SEQ ID NO: 3. In some embodiments, the β-hexosaminidase variant α subunit comprises the amino acid sequence according to SEQ ID NO: 3. In some embodiments, the β-hexosaminidase variant α subunit comprises at least 500 contiguous amino acids of an amino acid sequence according to SEQ ID NO: 3. In some embodiments, the β-hexosaminidase variant α subunit comprises at least 525 contiguous amino acids of an amino acid sequence according to SEQ ID NO: 3. In some embodiments, the β-hexosaminidase variant α subunit comprises at least 550 contiguous amino acids of an amino acid sequence according to SEQ ID NO: 3. In some embodiments, the β-hexosaminidase variant α subunit comprises at least 500 contiguous amino acids of an amino acid sequence according to SEQ ID NO: 3, and at least 95% sequence identity to the at least 500 contiguous amino acids. In some embodiments, the β-hexosaminidase variant α subunit comprises at least 525 contiguous amino acids of an amino acid sequence according to SEQ ID NO: 3, and at least 95% sequence identity to the at least 525 contiguous amino acids. In some embodiments, the β-hexosaminidase variant α subunit comprises at least 550 contiguous amino acids of an amino acid sequence according to SEQ ID NO: 3, and at least 950% sequence identity to the at least 550 contiguous amino acids. In some embodiments, the β-hexosaminidase variant α subunit comprises an amino acid sequence with at least 85% sequence identity to SEQ ID NO: 3. In some embodiments, the β-hexosaminidase variant α subunit homodimer comprises an increased mannose-6-phosphorylation (M6P) relative to a homodimer of SEQ ID NO: 6. In some embodiments, the β-hexosaminidase variant α subunit homodimer has at least 3 M6P sites occupied by M6P per homodimer. In some embodiments, the β-hexosaminidase variant α subunit homodimer has at least 4 M6P sites occupied by M6P per homodimer. In some embodiments, the β-hexosaminidase variant α subunit homodimer exhibits GM2 ganglioside hydrolysis activity in the presence of GM2-activator protein. In some embodiments, the cellular uptake of the β-hexosaminidase variant α subunit homodimer is increased by at least 2-fold, 5-fold, 10-fold, 20-fold, or 25-fold relative to a homodimer of SEQ ID NO: 6. In some embodiments, the cellular uptake of the variant the β-hexosaminidase variant α subunit homodimer is assessed by uptake of cation independent mannose-6-phosphate receptor (CI-MPR) in a competition assay of the homodimer and mannose-6-phosphate in human fibroblasts from SD patients. In some embodiments, the β-hexosaminidase variant α subunit homodimer has increased thermal stability relative to a heterodimer of SEQ ID NO: 1 and SEQ ID NO: 2. In some embodiments, the β-hexosaminidase variant α subunit homodimer has a melting point (Tm) of about 60° C. to about 63° C. In some embodiments, the β-hexosaminidase variant α subunit homodimer increases lifespan of a Hexb knockout mouse when administered at least age 80 days of age as compared to a heterodimer of SEQ ID NO: 1 and SEQ ID NO: 2 that is administered to a Hexb knockout mouse under substantially equivalent assay conditions and age. In some embodiments, the β-hexosaminidase variant α subunit homodimer increases lifespan by at least 2-fold of a Hexb knockout mouse when administered at least 80 days of age as compared to a heterodimer of SEQ ID NO: 1 and SEQ ID NO: 2 that is administered to a Hexb knockout mouse under substantially equivalent assay conditions and age.

### Recombinant β-Hexosaminidase Variant α Subunit Polynucleotide Compositions

Disclosed herein are polynucleotides that encode any recombinant β-hexosaminidase variant α subunit disclosed herein. The polynucleotide can be part of a vector, construct, or plasmid. In some embodiments, a nucleic acid encodes a recombinant β-hexosaminidase variant α subunit homodimer as disclosed herein. In some embodiments, a vector disclosed herein comprises the nucleic acid and one or more gene regulatory regions. In some embodiments, the polynucleotide comprises a nucleic acid sequence according to SEQ ID NO: 8. In some embodiments, the polynucleotide comprises a nucleic acid sequence that has at least 70%, 75%, 805 85%, 90%, 91%, 92%, 93%, 94%, 95%, 96%, 97%, 98%, or 99% sequence identity to the nucleic acid sequence according to SEQ ID NO: 8.

### Methods of Treatment

In some embodiments, are methods of treating, ameliorating the symptoms or slowing progression of TSD in a subject in need thereof comprising administering to the subject an effective amount of the β-hexosaminidase variant α subunit homodimers as described herein. In some embodiments, the subject has at least one symptom of TSD. In some embodiments, the subject has received a diagnosis of TSD. In some embodiments, the subject is in the early stages of TSD. In some embodiments, the subject is asymptomatic of TSD.

In some embodiments, the subject is administered the effective amount of the β-hexosaminidase variant α subunit homodimers as described herein within at least one year of first presenting with a symptom of TSD. In some embodiments, the subject is administered the effective amount of the β-hexosaminidase variant α subunit homodimers as described herein within at least 11 months of first presenting with a symptom of TSD. In some embodiments, the subject is administered the effective amount of the β-hexosaminidase variant α subunit homodimers as described herein within at least 10 months of first presenting with a symptom of TSD. In some embodiments, the subject is administered the effective amount of the β-hexosaminidase variant α subunit homodimers as described herein within at least 9 months of first presenting with a symptom of TSD. In some embodiments, the subject is administered the effective amount of the β-hexosaminidase variant α subunit homodimers as described herein within at least 8 months of first presenting with a symptom of TSD. In some embodiments, the subject is administered the effective amount of the β-hexosaminidase variant α subunit homodimers as described herein within at least 7 months of first presenting with a symptom of TSD. In some embodiments, the subject is administered the effective amount of the β-hexosaminidase variant α subunit homodimers as described herein within at least 6 months of first presenting with a symptom of TSD. In some embodiments, the subject is administered the effective amount of the β-hexosaminidase variant α subunit homodimers as described herein within at least 5 months of first presenting with a symptom of TSD. In some embodiments, the subject is administered the effective amount of the β-hexosaminidase variant α subunit homodimers as described herein within at least 4 months of first presenting with a symptom of TSD. In some embodiments, the subject is administered the effective amount of the β-hexosaminidase variant α subunit homodimers as described herein within at least 3 months of first presenting with a symptom of TSD. In some embodiments, the subject is administered the effective amount of the β-hexosaminidase variant α subunit homodimers as described herein within at least 2 months of first presenting with a symptom of TSD. In some embodiments, the subject is administered the effective amount of the β-hexosaminidase variant α subunit homodimers as described herein within at least 1 month of first presenting with a symptom of TSD. In some embodiments, the subject is administered the effective amount of the β-hexosaminidase variant α subunit homodimers as described herein within at least 3 weeks of first presenting with a symptom of TSD. In some embodiments, the subject is administered the effective amount of the β-hexosaminidase variant α subunit homodimers as described herein within at least 2 weeks of first presenting with a symptom of TSD. In some embodiments, the subject is administered the effective amount of the β-hexosaminidase variant α subunit homodimers as described herein within at least 1 week of first presenting with a symptom of TSD. In some embodiments, the subject is administered the effective amount of the β-hexosaminidase variant α subunit homodimers as described herein within at least 6 days of first presenting with a symptom of TSD. In some embodiments, the subject is administered the effective amount of the β-hexosaminidase variant α subunit homodimers as described herein within at least 5 days of first presenting with a symptom of TSD. In some embodiments, the subject is administered the effective amount of the β-hexosaminidase variant α subunit homodimers as described herein within at least 4 days of first presenting with a symptom of TSD. In some embodiments, the subject is administered the effective amount of the β-hexosaminidase variant α subunit homodimers as described herein within at least 3 days of first presenting with a symptom of TSD. In some embodiments, the subject is administered the effective amount of the β-hexosaminidase variant α subunit homodimers as described herein within at least 2 days of first presenting with a symptom of TSD. In some embodiments, the subject is administered the effective amount of the β-hexosaminidase variant α subunit homodimers as described herein within at least 1 day of first presenting with a symptom of TSD.

In some embodiments, the subject is administered the effective amount of the β-hexosaminidase variant α subunit homodimers as described herein during an asymptomatic stage of a disease described herein. In some embodiments, the subject is administered the effective amount of the β-hexosaminidase variant α subunit homodimers as described herein during a pre-symptomatic stage of a disease described herein. In some embodiments, the subject is administered the effective amount of the β-hexosaminidase variant α subunit homodimers as described herein during an early (pre-symptomatic) stage of a disease described herein. In some embodiments, the subject is administered the effective amount of the β-hexosaminidase variant α subunit homodimers as described herein during an late (moderate to severe decline) stage of a disease described herein.

In some embodiments, the subject is pre-symptomatic for TSD. In some embodiments, the subject has a TSD-associated mutation. In some embodiments, the subject has an elevated level of a biomarker of TSD relative to a control level of the biomarker of TSD. In some embodiments, the elevated level of a biomarker of TSD is a measurement of the biomarker of TSD obtained from a sample from the subject. In some embodiments, the control level of the biomarker of TSD is a measurement of the biomarker of TSD from a sample from a subject that does not have a TSD-associated mutation. In some embodiments, the sample from the subject and the sample from the subject that does not have a TSD-associated mutation are quantified using liquid chromatography and mass spectrometry. In some embodiments, the sample from the subject and the sample from the subject that does not have a TSD-associated mutation are obtained from the CSF or blood. In some embodiments, the sample from the subject and the sample from the subject that does not have a TSD-associated mutation are blood plasma samples. In some embodiments, the elevated level of the biomarker of the TSD is elevated by at least 100% to 4000% relative to the control level of the biomarker of TSD. In some embodiments, the elevated level of the biomarker of the TSD is elevated by at least 2× to 100× relative to the control level of the biomarker of TSD. In some embodiments, the elevated level of the biomarker of the TSD is elevated by at least 2×, 3×, 4×, 5×, 6×, 7×, 8×, 9×, 10×, 15×, 20×, 25×, 30×, 35×, 40×, 45×, 50×, 55×, 60×, 65×, 70×, 75×, 80×, 85×, 90×, 95×, 100×, 110×, 120×, 130×, 140×, 150×, 160×, 170×, 180×, 190×, 200×, 250×, 300×, 350×, 400×, 450×, 500×, 550×, 600×, 650×, 700×, 750×, 800×, 850×, 900×, 950×, 1000×, 2000×, 3000×, 4000×, 5000×, or 10000× relative to the control level of the biomarker of TSD. In some embodiments, the elevated level of the biomarker of the TSD is elevated by at least about 5%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, 200%, 225%, 250%, 275%, 300%, 325%, 350%, 375%, 400%, 425%, 450%, 475%, 500%, 600%, 700%, 800%, 900%, 1000%, 2000%, 3000%, 4000%, 5000%, or 10000% relative to the control level of the biomarker of TSD. In some embodiments, the biomarker is GM2, GA2, A2G0′ containing beta-linked terminal N-acetyl-D-hexosamine, BMP(22:6) phospholipid (Bis[monoacylglycero]phosphate)), neurofilament light chain or a free glycan. In some embodiments, the biomarker is neurofilament light chain.

In some embodiments, are methods of treating, ameliorating the symptoms or slowing progression of SD in a subject in need thereof disease comprising administering to the subject an effective amount of the β-hexosaminidase variant α subunit homodimers as described herein. In some embodiments, the subject has at least one symptom of SD. In some embodiments, the subject has received a diagnosis of SD. In some embodiments, the subject is in the early stages of SD. In some embodiments, the subject is asymptomatic of SD.

In some embodiments, the subject is administered the effective amount of the β-hexosaminidase variant α subunit homodimers as described herein within at least 6 months of first presenting with a symptom of SD. In some embodiments, the subject is administered the effective amount of the β-hexosaminidase variant α subunit homodimers as described herein within at least 5 months of first presenting with a symptom of SD. In some embodiments, the subject is administered the effective amount of the β-hexosaminidase variant α subunit homodimers as described herein within at least 4 months of first presenting with a symptom of SD. In some embodiments, the subject is administered the effective amount of the β-hexosaminidase variant α subunit homodimers as described herein within at least 3 months of first presenting with a symptom of SD. In some embodiments, the subject is administered the effective amount of the β-hexosaminidase variant α subunit homodimers as described herein within at least 2 months of first presenting with a symptom of SD. In some embodiments, the subject is administered the effective amount of the β-hexosaminidase variant α subunit homodimers as described herein within at least 1 month of first presenting with a symptom of SD. In some embodiments, the subject is administered the effective amount of the β-hexosaminidase variant α subunit homodimers as described herein within at least 3 weeks of first presenting with a symptom of SD. In some embodiments, the subject is administered the effective amount of the β-hexosaminidase variant α subunit homodimers as described herein within at least 2 weeks of first presenting with a symptom of SD. In some embodiments, the subject is administered the effective amount of the β-hexosaminidase variant α subunit homodimers as described herein within at least 1 week of first presenting with a symptom of SD.

In some embodiments, the subject has a SD-associated mutation. In some embodiments, the subject has an elevated level of a biomarker of SD relative to a control level of the biomarker of SD. In some embodiments, the elevated level of a biomarker of SD is a measurement of the biomarker of SD obtained from a sample from the subject. In some embodiments, the control level of the biomarker of SD is a measurement of the biomarker of SD from a sample from a subject that does not have a SD-associated mutation. In some embodiments, the sample from the subject and the sample from the subject that does not have a SD-associated mutation are quantified using liquid chromatography and mass spectrometry. In some embodiments, the sample from the subject and the sample from the subject that does not have a SD-associated mutation are obtained from the CSF or blood. In some embodiments, the sample from the subject and the sample from the subject that does not have a SD-associated mutation are blood plasma samples. In some embodiments, the elevated level of the biomarker of the SD is elevated by at least 100% to 4000% relative to the control level of the biomarker of SD. In some embodiments, the elevated level of the biomarker of the SD is elevated by at least 2× to 100× relative to the control level of the biomarker of SD. In some embodiments, the elevated level of the biomarker of the SD is elevated by at least 2×, 3×, 4×, 5×, 6×, 7×, 8×, 9×, 10×, 15×, 20×, 25×, 30×, 35×, 40×, 45×, 50×, 55×, 60×, 65×, 70×, 75×, 80×, 85×, 90×, 95×, 100×, 10×, 120×, 130×, 140×, 150×, 160×, 170×, 180×, 190×, 200×, 250×, 300×, 350×, 400×, 450×, 500×, 550×, 600×, 650×, 700×, 750×, 800×, 850×, 900×, 950×, 1000×, 2000×, 3000×, 4000×, 5000×, or 10000× relative to the control level of the biomarker of SD. In some embodiments, the elevated level of the biomarker of the SD is elevated by at least about 5%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, 200%, 225%, 250%, 275%, 300%, 325%, 350%, 375%, 400%, 425%, 450%, 475%, 500%, 600%, 700%, 800%, 900%, 1000%, 2000%, 3000%, 4000%, 5000%, or 10000% relative to the control level of the biomarker of SD. In some embodiments, the biomarker is GM2, GA2, A2G0′ containing beta-linked terminal N-acetyl-D-hexosamine, BMP(22:6) phospholipid (Bis[monoacylglycero]phosphate)), neurofilament light chain or a free glycan. In some embodiments, the biomarker is neurofilament light chain.

The present disclosure provides a method for treating, ameliorating the symptoms of, or slowing the disease progression of TSD, SD, or GM2 gangliosidosis in a subject. A subject can be, for example, a patient. TSD is caused by a mutation in the gene that encodes the α subunit of hexosaminidase enzyme located on chromosome 15. Loss of hexosaminidase enzyme activity causes an accumulation of GM2 gangliosides which eventually causes neuronal cell death. SD is caused by a mutation in the gene that encodes the β subunit of hexosaminidase enzyme located on chromosome 5. Loss of hexosaminidase enzyme activity causes an accumulation of GM2 gangliosides which eventually causes neuronal cell death.

As disclosed herein, treatment of TSD or SD can comprise decreasing the lysosomal storage of GM2 gangliosides (or GM2 ganglioside precursor molecules) in various tissues in a subject. The issues in which lysosomal storage of GM2 gangliosides can be reduced include, for example, brain, spinal cord neurons, or peripheral target tissues. A method disclosed herein can reduce lysosomal storage of GM2 or one or more of GM2 precursors by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100% as compared to a control. A method disclosed herein can decrease lysosomal storage GM2 or one or more of GM2 precursors by at least 1-fold, 2-fold, 3-fold, 4-fold, 5-fold, 6-fold, 7-fold, 8-fold, 9-fold, 10-fold or more as compared to a control.

As disclosed herein, treatment of TSD or SD can comprise increased hexosaminidase enzyme activity in various tissues. The increase in hexosaminidase enzyme activity can be increased by, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 200%, about 300%, about 400%, about 500%, about 600%, about 700%, about 800%, about 900%, or about 1000% as compared to a control. The increase in hexosaminidase enzyme activity can be increased by at least 1-fold, 2-fold, 3-fold, 4-fold, 5-fold, 6-fold, 7-fold, 8-fold, 9-fold, 10-fold or more as compared to a control. The increased enzymatic activity is at least approximately 10 nmol/hr/mg, 20 nmol/hr/mg, 40 nmol/hr/mg, 50 nmol/hr/mg, 60 nmol/hr/mg, 70 nmol/hr/mg, 80 nmol/hr/mg, 90 nmol/hr/mg, 100 nmol/hr/mg, 150 nmol/hr/mg, 200 nmol/hr/mg, 250 nmol/hr/mg, 300 nmol/hr/mg, 350 nmol/hr/mg, 400 nmol/hr/mg, 450 nmol/hr/mg, 500 nmol/hr/mg, 550 nmol/hr/mg, 600 nmol/hr/mg or more.

### Enzyme Replacement Therapy

Enzyme Replacement Therapy (ERT) can involve delivering wild-type or enzymatically active variant versions of lysosomal enzymes that are reduced or missing in particular lysosomal storage diseases. ERT can involve infusion of a missing enzyme into the bloodstream. As the blood perfuses patient tissues, the enzyme can be taken up by cells and transported to the lysosome, where the enzyme acts to eliminate material that has accumulated in the lysosomes due to the enzyme deficiency. For lysosomal enzyme replacement therapy to be effective, the therapeutic enzyme must be delivered to lysosomes in the appropriate cells in tissues where the storage defect exists.

For example, ERT for TSD or SD can involve the administration of wild-type or enzymatically active variants of hexosaminidase enzyme to a subject. To be effective, a variant β-hexosaminidase enzyme disclosed herein should be able to target neuronal cells, which can be accomplished via intrathecal or intracerebroventricular administration. ERT can also require that the administered lysosomal enzyme be taken up and delivered to the lysosomal compartment of the cell, which can require that the enzyme have enough M6P sites to be taken up by the cell via the cation-independent mannose 6-phosphate (M6P) receptor (CI-MPR).

To avoid an immune system-mediated reaction to the ERT, a subject may be administered an additional therapy in combination with the ERT. Such an additional therapy can be, for example, an immune tolerance induction therapy. An additional therapy can be, for example, cyclosporin A (CsA) or azathioprine (Aza).

### Gene Therapy

A method disclosed herein can comprise gene therapy for the treatment of TSD and SD. For example, a vector comprising a variant β-hexosaminidase enzyme disclosed herein can be used for gene therapy. In some embodiments, the vector is an adeno-associated viral (AAV) vector. In some embodiments, the vector is able to cross the blood brain vector, such as AAV9. In some embodiments, the vector is a lentiviral vector. For example, a lentiviral vector can be used to transfer a nucleic acid molecule encoding a variant hexosaminidase α subunit into hematopoietic stem cells, which then can be administered to a subject as an ex vivo gene therapy.

A vector disclosed herein can comprise adeno-associated virus (AAV) inverted terminal repeats (ITR) at both the 5′ and 3′ end of the vector.

In some embodiments, the present disclosure provides a method of ameliorating the symptoms or slowing disease progression of TSD in a subject having TSD comprising administering an effective amount of the AAV viral particle comprising a vector encoding a recombinant β-hexosaminidase enzyme to the subject having TSD.

In some embodiments, the present disclosure provides a method of ameliorating the symptoms or slowing disease progression of SD in a subject having TSD comprising administering an effective amount of the AAV viral particle comprising a vector encoding a recombinant β-hexosaminidase enzyme to the subject having SD.

### Pharmaceutical Compositions

A pharmaceutical composition of the disclosure can be a combination of any pharmaceutical compounds described herein with other chemical components, such as carriers, stabilizers, diluents, dispersing agents, suspending agents, thickening agents, and/or excipients. The pharmaceutical composition facilitates administration of the compound to an organism. In some embodiments, the recombinant β-hexosaminidase variant α subunit further comprises a detectable label, a therapeutic agent, or a pharmacokinetic modifying moiety. In some embodiments, the detectable label comprises a fluorescent label, a radiolabel, an enzyme, a nucleic acid probe, or a contrast agent.

Pharmaceutical formulations for administration can include aqueous solutions of the active compounds in water-soluble form. Suspensions of the active compounds can be prepared as oily injection suspensions. Suitable lipophilic solvents or vehicles include fatty oils such as sesame oil, or synthetic fatty acid esters, such as ethyl oleate or triglycerides, or liposomes. Aqueous injection suspensions can contain substances which increase the viscosity of the suspension, such as sodium carboxymethyl cellulose, sorbitol, or dextran. The suspension can also contain suitable stabilizers or agents which increase the solubility of the compounds to allow for the preparation of highly concentrated solutions. The active ingredient can be in powder form for constitution with a suitable vehicle, for example, sterile pyrogen-free water, before use.

Pharmaceutical compositions can be formulated using one or more physiologically-acceptable carriers comprising excipients and auxiliaries, which facilitate processing of the active compounds into preparations that can be used pharmaceutically. Formulation can be modified depending upon the route of administration chosen. Pharmaceutical compositions comprising compounds described herein can be manufactured, for example, by mixing, dissolving, emulsifying, encapsulating, entrapping, or compression processes.

The pharmaceutical compositions can include at least one pharmaceutically-acceptable carrier, diluent, or excipient and compounds described herein as free-base or pharmaceutically-acceptable salt form. Pharmaceutical compositions can contain solubilizers, stabilizers, tonicity enhancing agents, buffers, and preservatives.

Non-limiting examples of pharmaceutically-acceptable excipients suitable for use in the disclosure include binding agents, disintegrating agents, anti-adherents, anti-static agents, surfactants, anti-oxidants, coating agents, coloring agents, plasticizers, preservatives, suspending agents, emulsifying agents, anti-microbial agents, spheronization agents, and any combination thereof.

A pharmaceutical composition disclosed herein can be formulated in an artificial cerebrospinal fluid (CSF) formulation. The CSF formulation can comprise, for example, sodium phosphate, sodium chloride, potassium chloride, magnesium chloride, and calcium chloride. Disclosed herein is a pharmaceutical composition comprising one or more pharmaceutically acceptable excipients, wherein the one or more pharmaceutically acceptable excipients comprise sodium phosphate, sodium chloride, potassium chloride, magnesium chloride, and calcium chloride. Disclosed herein is a pharmaceutical composition, wherein the pharmaceutical composition comprises 1 mM sodium phosphate, 148 sodium chloride, 3 mM potassium chloride, 0.8 mM magnesium chloride, 1.4 mM calcium chloride, pH 7.2.

### Administration of a Pharmaceutical Composition Disclosed Herein

In practicing the methods of treatment or use provided herein, therapeutically-effective amounts of the recombinant β-hexosaminidase variant α subunit homodimers described herein are administered in pharmaceutical compositions to a subject having a disease or condition to be treated. In some embodiments, the subject is a mammal such as a human. A therapeutically-effective amount can vary widely depending on the severity of the disease, the age and relative health of the subject, the potency of the compounds used, and other factors. In some embodiments, a pharmaceutical composition is administered to the subject prior to the subject presenting with any symptoms of the disease. In some embodiments, a pharmaceutical composition disclosed herein is administered early in the onset of disease in the subject. In some embodiments, administration of a pharmaceutical composition disclosed herein early in the onset of the disease in subject demonstrates higher efficacy than administration of the pharmaceutical at a late stage of disease.

The recombinant β-hexosaminidase variant α subunit homodimers are administered by any appropriate route. In various embodiments, the recombinant β-hexosaminidase variant α subunit homodimer is administered intravenously. In other embodiments, a therapeutic protein is administered by direct administration to a target tissue, such as heart or muscle (e.g., intramuscular), or nervous system (e.g., direct injection into the brain; intraventricularly; intrathecally).

In some embodiments, a pharmaceutical composition disclosed herein is administered by introduction into the central nervous system of the subject, for example, into the cerebrospinal fluid of the subject. In some embodiments, the pharmaceutical composition is introduced intrathecally, intraventricularly, or intracerebroventricularly into a cerebral ventricle space. In some embodiments, a pharmaceutical composition disclosed herein is administered intracerebroventricularly.

For example, a pharmaceutical composition disclosed herein can be administered using an Ommaya reservoir. In an Ommaya reservoir, a ventricular tube is inserted through a hole formed in the anterior horn and is connected to an Ommaya reservoir installed under the scalp, and the reservoir is subcutaneously punctured to intrathecally deliver the particular enzyme being replaced, which is injected into the reservoir. A pharmaceutical composition disclosed herein can be intrathecally given, for example by a single injection. Administration of a pharmaceutical composition disclosed herein can be performed via direct injection of the composition or by the use of infusion pumps.

In some embodiments, a pharmaceutical composition disclosed herein is administered by lateral cerebroventricular injection into the brain of a subject. The injection can be made, for example, through a burr hole made in the subject's skull. In some embodiments, a pharmaceutical composition disclosed herein is administered through a surgically inserted shunt into the cerebral ventricle of a subject. For example, the injection can be made into the lateral ventricles, which are larger, even though injection into the third and fourth smaller ventricles can also be made.

In some embodiments, a pharmaceutical composition disclosed herein is delivered to one or more surface or shallow tissues of the brain, cerebrum, or spinal cord. In some embodiments, the targeted surface or shallow tissues of the cerebrum are selected from pia mater tissues, cerebral cortical ribbon tissues, hippocampus, Virchow Robin space, blood vessels within the VR space, the hippocampus, portions of the hypothalamus on the inferior surface of the brain, the optic nerves and tracts, the olfactory bulb and projections, and combinations thereof.

In some embodiments, a pharmaceutical composition disclosed herein is delivered to one or more deep tissues of the cerebrum or spinal cord. In some embodiments, the targeted surface or shallow tissues of the cerebrum or spinal cord are located 4 mm, 5 mm, 6 mm, 7 mm, 8 mm, 9 mm, or 10 mm below the surface of the cerebrum. In some embodiments, targeted deep tissues of the cerebrum include the cerebral cortical ribbon. In some embodiments, targeted deep tissues of the cerebrum include one or more of the diencephalon, the hypothalamus, thalamus, prethalamus, subthalamus, metencephalon, lentiform nuclei, the basal ganglia, caudate, putamen, amygdala, globus pallidus, and combinations thereof.

In some embodiments, a targeted surface or shallow tissue of the spinal cord contains pia matter and/or the tracts of white matter. In some embodiments, a targeted deep tissue of the spinal cord contains spinal cord grey matter and/or ependymal cells.

In some embodiments, a pharmaceutical composition disclosed herein is delivered to one or more tissues of the cerebellum. In some embodiments, the targeted one or more tissues of the cerebellum are tissues of the molecular layer, tissues of the Purkinje cell layer, tissues of the Granular cell layer, cerebellar peduncles, and combination thereof. In some embodiments, a therapeutic agent disclosed herein is delivered to one or more deep tissues of the cerebellum including, but not limited to, tissues of the Purkinje cell layer, tissues of the Granular cell layer, deep cerebellar white matter tissue (e.g., deep relative to the Granular cell layer), and deep cerebellar nuclei tissue.

In some embodiments, a pharmaceutical composition disclosed herein is delivered to one or more tissues of the brainstem. In some embodiments, the targeted one or more tissues of the brainstem include brain stem white matter tissue and/or brain stem nuclei tissue.

In some embodiments, a pharmaceutical composition disclosed herein is delivered to various brain tissues including, but not limited to, gray matter, white matter, periventricular areas, pia-arachnoid, meninges, neocortex, cerebellum, deep tissues in cerebral cortex, molecular layer, caudate/putamen region, midbrain, deep regions of the pons or medulla, and combinations thereof.

In some embodiments, a pharmaceutical composition disclosed herein is delivered to various cells in the brain including, but not limited to, neurons of the brain stem, neurons of the spinal cord, glial cells, perivascular cells and/or meningeal cells. In some embodiments, a therapeutic protein is delivered to oligodendrocytes of deep white matter.

A pharmaceutical composition disclosed herein can be administered alone, or in conjunction with other agents, such as antihistamines (e.g., diphenhydramine) or immunosuppressants or other immunotherapeutic agents. A pharmaceutical composition described herein can be administered before, at the same time, or after an additional therapeutic agent. For example, the additional therapeutic agent can be mixed into a composition containing the therapeutic protein, and thereby administered contemporaneously with the therapeutic protein. The agent can be administered separately (e.g., not admixed), but within a short time frame (e.g., within 24 hours) of administration of the therapeutic protein. Non-limiting examples of pharmaceutically active agents suitable for combination with compositions of the disclosure include anti-infectives, i.e., aminoglycosides, antiviral agents, antimicrobials, anti-cholinergics/anti-spasmotics, antidiabetic agents, antihypertensive agents, anti-neoplastics, cardiovascular agents, central nervous system agents, coagulation modifiers, hormones, immunologic agents, and immunosuppressive agents.

The homodimer comprising a recombinant β-hexosaminidase variant α subunit disclosed herein can be administered in a therapeutically effective amount. The recombinant β-hexosaminidase variant α subunit disclosed herein can be formulated in a pharmaceutical composition at about 1 mg/mL, about 2 mg/mL, about 3 mg/mL, about 4 mg/mL, about 5 mg/mL, about 6 mg/mL, about 7 mg/mL, about 8 mg/mL, about 9 mg/mL, about 10 mg/mL, about 15 mg/mL, about 20 mg/mL, about 25 mg/mL, about 30 mg/mL, about 35 mg/mL, about 40 mg/mL, about 45 mg/mL, or about 50 mg/mL. Disclosed herein is a pharmaceutical composition, wherein the homodimer comprising the recombinant β-hexosaminidase variant α subunit is formulated at a concentration of about 20 mg/ml.

The effective dose for a particular individual can be varied (e.g., increased or decreased) over time, depending on the needs of the individual. For example, in times of physical illness or stress, or if disease symptoms worsen, the dosage amount can be increased.

A pharmaceutical composition disclosed herein can be administered after the onset of symptoms of any condition disclosed herein. A pharmaceutical composition disclosed herein can demonstrate increased efficacy if administered at a late stage of disease, rather than at an early stage of disease.

In some embodiments, a pharmaceutical composition disclosed herein is administered bimonthly, monthly, twice monthly, triweekly, biweekly, weekly, twice weekly, thrice weekly, or daily. In some embodiments, a pharmaceutical composition disclosed herein is administered weekly for 12 weeks and then administered biweekly.

### Kits for Use in a Method Disclosed Herein

Compounds and compositions disclosed herein can be packaged as a kit. In some embodiments, the present disclosure provides a kit comprising a compound disclosed herein, and written instructions on use of the kit in the treatment of a condition described herein.

A kit disclosed herein can comprise a β-hexosaminidase variant α subunit homodimer as described herein for use in the treatment of a lysosomal storage disease and a lysosomal targeting moiety, in a dose and form suitable for administration to a subject. In some embodiments, the kit can comprise a device for delivering the enzyme intrathecally.

The kit disclosed herein can comprise instructions for the intrathecal administration of a pharmaceutical composition disclosed herein. In some embodiments, a kit disclosed herein comprises a catheter or other device for intrathecal administration of a pharmaceutical composition disclosed herein. For example, a kit disclosed herein can comprise a catheter preloaded with 0.001-0.01 mg, 0.01-0.1 mg, 0.1-1.0 mg, 1.0-10 mg, 10-100 mg, or more of a therapeutic protein comprising a lysosomal enzyme and lysosomal targeting moiety, such as HexD3 or HexA, in a pharmaceutically acceptable formulation.

### Embodiments

Embodiment 1 comprises an recombinant β-hexosaminidase variant α subunit that forms a β-hexosaminidase variant α subunit homodimer comprising one or more amino acid sequence substitutions or deletions at positions corresponding to S184, P209, N228, P229, V230, T231, P429, K432, D433, 1436, N466, S491, L493, T494, F495, E498, L508, Q513, N518, V519, F521, and E523 of native β-hexosaminidase α-subunit of SEQ ID NO:1 and further comprising one or more amino acid sequence elements that increase cellular uptake of the β-hexosaminidase variant α subunit homodimer relative to a homodimer of SEQ ID NO: 6.

Embodiment 2 comprises the recombinant β-hexosaminidase variant α subunit of embodiment 1, wherein the one or more amino acid sequence elements that increase cellular uptake are selected from the group consisting of amino acid sequence substitutions, additions, or deletions relative to the native β-hexosaminidase α-subunit of SEQ ID NO:1.

Embodiment 3 comprises the recombinant β-hexosaminidase variant α subunit of any of embodiments 1-2, wherein the β-hexosaminidase variant α subunit comprises one or more amino acid substitutions or deletions corresponding to S184K, P209Q, N228S, P229Δ, V230L, T231S, P429Q, K432R, D433K, I436K, N466A, S491R, L493M, T494D, F495D, E498D, L508V, Q513V, N518Y, V519A, F521Y, and E523N relative to the native β-hexosaminidase α-subunit of SEQ ID NO: 1.

Embodiment 4 comprises the recombinant β-hexosaminidase variant α subunit of any of embodiments 1-3, wherein the β-hexosaminidase variant α subunit comprises at least five amino acid substitutions or deletions corresponding to S184K, P209Q, N228S, P229Δ, V230L, T231S, P429Q, K432R, D433K, I436K, N466A, S491R, L493M, T494D, F495D, E498D, L508V, Q513V, N518Y, V519A, F521Y, and E523N relative to the native β-hexosaminidase α-subunit of SEQ ID NO: 1.

Embodiment 5 comprises the recombinant β-hexosaminidase variant α subunit of any of embodiments 1-4, wherein the β-hexosaminidase variant α subunit comprises at least ten amino acid substitutions or deletions corresponding to S184K, P209Q, N228S, P229Δ, V230L, T231S, P429Q, K432R, D433K, I436K, N466A, S491R, L493M, T494D, F495D, E498D, L508V, Q513V, N518Y, V519A, F521Y, and E523N relative to the native β-hexosaminidase α-subunit of SEQ ID NO: 1.

Embodiment 6 comprises the recombinant β-hexosaminidase variant α subunit of any of embodiments 1-5, wherein the β-hexosaminidase variant α subunit comprises at least fifteen amino acid substitutions or deletions corresponding to S184K, P209Q, N228S, P229Δ, V230L, T231S, P429Q, K432R, D433K, I436K, N466A, S491R, L493M, T494D, F495D, E498D, L508V, Q513V, N518Y, V519A, F521Y, and E523N relative to the native β-hexosaminidase α-subunit of SEQ ID NO:1.

Embodiment 7 comprises the recombinant β-hexosaminidase variant α subunit of any of embodiments 1-6, wherein the β-hexosaminidase variant α subunit comprises at least twenty amino acid substitutions or deletions corresponding to S184K, P209Q, N228S, P229Δ, V230L, T231S, P429Q, K432R, D433K, I436K, N466A, S491R, L493M, T494D, F495D, E498D, L508V, Q513V, N518Y, V519A, F521Y, and E523N relative to the native β-hexosaminidase α-subunit of SEQ ID NO:1.

Embodiment 8 comprises the recombinant β-hexosaminidase variant α subunit of any of embodiments 1-7, wherein the β-hexosaminidase variant α subunit comprises amino acid substitutions or deletions corresponding to S184K, P209Q, N228S, P229Δ, V230L, T231S, P429Q, K432R, D433K, I436K, N466A, S491R, L493M, T494D, F495D, E498D, L508V, Q513V, N518Y, V519A, F521Y, and E523N relative to the native β-hexosaminidase α subunit of SEQ ID NO:1.

Embodiment 9 comprises the recombinant β-hexosaminidase variant α subunit of any of embodiments 1-8, wherein the β-hexosaminidase variant α subunit comprises a first amino acid sequence comprising at least 85% sequence identity to SEQ ID NO: 11 and the one or more amino acid sequence elements that increase cellular uptake of the β-hexosaminidase variant α subunit relative to a homodimer of SEQ ID NO: 6 comprises a second amino acid sequence.

Embodiment 10 comprises the recombinant β-hexosaminidase variant α subunit of any of embodiments 1-9, wherein the first amino acid sequence comprises at least 90% sequence identity to SEQ ID NO: 11.

Embodiment 11 comprises the recombinant β-hexosaminidase variant α subunit of embodiment 9 or embodiment 10, wherein the first amino acid sequence comprises at least 95% sequence identity to SEQ ID NO: 11.

Embodiment 12 comprises the recombinant β-hexosaminidase variant α subunit of any of embodiments 9-11, wherein the first amino acid sequence comprises at least 99% sequence identity to SEQ ID NO: 11.

Embodiment 13 comprises the recombinant β-hexosaminidase variant α subunit of any of embodiments 9-12, wherein the first amino acid sequence comprises the amino acid sequence of SEQ ID NO: 11.

Embodiment 14 comprises the recombinant β-hexosaminidase variant α subunit of any of embodiments 9-13, wherein the second amino acid sequence is N-terminal to the first amino acid sequence.

Embodiment 15 comprises the recombinant β-hexosaminidase variant α subunit of any of embodiments 9-14, wherein the second amino acid sequence comprises at least 20 contiguous amino acid residues of SEQ ID NO: 2.

Embodiment 16 comprises the recombinant β-hexosaminidase variant α subunit of any of embodiments 9-14, wherein the second amino acid sequence comprises at least 100 contiguous amino acid residues of SEQ ID NO: 12.

Embodiment 17 comprises the recombinant β-hexosaminidase variant α subunit of any of embodiments 9-14, wherein the second amino acid sequence comprises at least 100 contiguous amino acid residues of SEQ ID NO: 13.

Embodiment 18 comprises the recombinant β-hexosaminidase variant α subunit of any of embodiments 9-14 and 17, wherein the second amino acid sequence comprises at least 150 contiguous amino acid residues of SEQ ID NO: 13.

Embodiment 19 comprises the recombinant β-hexosaminidase variant α subunit of any of embodiments 9-14 and 17-18, wherein the second amino acid sequence comprises at least 85% sequence identity to SEQ ID NO: 13.

Embodiment 20 comprises the recombinant β-hexosaminidase variant α subunit of any of embodiments 9-14 and 17-19, wherein the second amino acid sequence comprises at least 90% sequence identity to SEQ ID NO: 13.

Embodiment 21 comprises the recombinant β-hexosaminidase variant α subunit of any of embodiments 9-14 and 17-20, wherein the second amino acid sequence comprises at least 95% sequence identity to SEQ ID NO: 13.

Embodiment 22 comprises the recombinant β-hexosaminidase variant α subunit of any of embodiments 9-14, wherein the second amino acid sequence comprises the amino acid sequence of SEQ ID NO: 13.

Embodiment 23 comprises the recombinant β-hexosaminidase variant α subunit of embodiment 1, wherein the β-hexosaminidase variant α subunit comprises an amino acid sequence with at least 90% sequence identity to SEQ ID NO: 3.

Embodiment 24 comprises the recombinant β-hexosaminidase variant α subunit of embodiment 23, wherein the β-hexosaminidase variant α subunit comprises an amino acid sequence with at least 95% sequence identity to SEQ ID NO: 3.

Embodiment 25 comprises the recombinant β-hexosaminidase variant α subunit of embodiment 23 or embodiment 24, wherein the β-hexosaminidase variant α subunit comprises an amino acid sequence with at least 99% sequence identity to SEQ ID NO: 3.

Embodiment 26 comprises the recombinant β-hexosaminidase variant α subunit of any of embodiments 23-25, wherein the β-hexosaminidase variant α subunit comprises the amino acid sequence according to SEQ ID NO: 3.

Embodiment 27 comprises the recombinant β-hexosaminidase variant α subunit of any of embodiments 23-26, wherein the β-hexosaminidase variant α subunit comprises at least 500 contiguous amino acids of an amino acid sequence according to SEQ ID NO: 3.

Embodiment 28 comprises the recombinant β-hexosaminidase variant α subunit of any of embodiments 23-27, wherein the β-hexosaminidase variant α subunit comprises at least 525 contiguous amino acids of an amino acid sequence according to SEQ ID NO: 3.

Embodiment 29 comprises the recombinant β-hexosaminidase variant α subunit of any of embodiments 23-28, wherein the β-hexosaminidase variant α subunit comprises at least 550 contiguous amino acids of an amino acid sequence according to SEQ ID NO: 3.

Embodiment 30 comprises the recombinant β-hexosaminidase variant α subunit of any of embodiments 23-29, wherein the β-hexosaminidase variant α subunit comprises at least 500 contiguous amino acids of an amino acid sequence according to SEQ ID NO: 3, and at least 95% sequence identity to the at least 500 contiguous amino acids.

Embodiment 31 comprises the recombinant β-hexosaminidase variant α subunit of any of embodiments 23-30, wherein the β-hexosaminidase variant α subunit comprises at least 525 contiguous amino acids of an amino acid sequence according to SEQ ID NO: 3, and at least 95% sequence identity to the at least 525 contiguous amino acids.

Embodiment 32 comprises the recombinant β-hexosaminidase variant α subunit of any of embodiments 23-31, wherein the β-hexosaminidase variant α subunit comprises at least 550 contiguous amino acids of an amino acid sequence according to SEQ ID NO: 3, and at least 95% sequence identity to the at least 550 contiguous amino acids.

Embodiment 33 comprises the recombinant β-hexosaminidase variant α subunit of any of embodiments 23-32, wherein the β-hexosaminidase variant α subunit comprises an amino acid sequence with at least 85% sequence identity to SEQ ID NO: 3.

Embodiment 34 comprises the recombinant β-hexosaminidase variant α subunit of any of embodiments 1-33, wherein the β-hexosaminidase variant α subunit homodimer comprises an increased mannose-6-phosphorylation (M6P) relative to a homodimer of SEQ ID NO: 6.

Embodiment 35 comprises the recombinant β-hexosaminidase variant α subunit of embodiment 34, wherein the β-hexosaminidase variant α subunit homodimer has at least 3 M6P sites occupied by M6P per homodimer.

Embodiment 36 comprises the recombinant β-hexosaminidase variant α subunit of embodiment 34 or embodiment 35, wherein the β-hexosaminidase variant α subunit homodimer has at least 4 M6P sites occupied by M6P per homodimer.

Embodiment 37 comprises the recombinant β-hexosaminidase variant α subunit of any of embodiments 1-36, wherein the β-hexosaminidase variant α subunit homodimer exhibits GM2 ganglioside hydrolysis activity in the presence of GM2-activator protein.

Embodiment 38 comprises the recombinant β-hexosaminidase variant α subunit of any of embodiments 1-37, wherein the cellular uptake of the β-hexosaminidase variant α subunit homodimer is increased by at least 2-fold, 5-fold, 10-fold, 20-fold, or 25-fold relative to a homodimer of SEQ ID NO: 6.

Embodiment 39 comprises the recombinant β-hexosaminidase variant α subunit of embodiment 38, wherein the cellular uptake of the variant the β-hexosaminidase variant α subunit homodimer is assessed by uptake of cation independent mannose-6-phosphate receptor (CI-MPR) in a competition assay of the homodimer and mannose-6-phosphate in human fibroblasts from SD patients.

Embodiment 40 comprises the recombinant β-hexosaminidase variant α subunit of any of embodiments 1-9, wherein the β-hexosaminidase variant α subunit homodimer has increased thermal stability relative to a heterodimer of SEQ ID NO: 1 and SEQ ID NO: 2.

Embodiment 41 comprises the recombinant β-hexosaminidase variant α subunit of embodiment 40, wherein the β-hexosaminidase variant α subunit homodimer has a melting point (Tm) of about 60° C. to about 63° C.

Embodiment 42 comprises the recombinant β-hexosaminidase variant α subunit of any of embodiments 1-41, wherein the β-hexosaminidase variant α subunit homodimer increases lifespan of a Hexb knockout mouse when administered at least age 80 days of age as compared to a heterodimer of SEQ ID NO: 1 and SEQ ID NO: 2 that is administered to a Hexb knockout mouse under substantially equivalent assay conditions and age.

Embodiment 43 comprises the recombinant β-hexosaminidase variant α subunit of any of embodiments 1-42, wherein the β-hexosaminidase variant α subunit homodimer increases lifespan by at least 2-fold of a Hexb knockout mouse when administered at least 80 days of age as compared to a heterodimer of SEQ ID NO: 1 and SEQ ID NO: 2 that is administered to a Hexb knockout mouse under substantially equivalent assay conditions and age.

Embodiment 44 comprises a homodimer comprising the recombinant β-hexosaminidase variant α subunit according to any one of embodiments 1-43.

Embodiment 45 comprises a pharmaceutical composition comprising the homodimer according to embodiment 44, and one or more pharmaceutically acceptable excipients.

Embodiment 46 comprises the pharmaceutical composition of embodiment 45, wherein the one or more pharmaceutically acceptable excipients comprise sodium phosphate, sodium chloride, potassium chloride, magnesium chloride, and calcium chloride.

Embodiment 47 comprises the pharmaceutical composition of embodiment 46, comprising 1 mM sodium phosphate, 148 sodium chloride, 3 mM potassium chloride, 0.8 mM magnesium chloride, 1.4 mM calcium chloride, pH 7.2.

Embodiment 48 comprises the pharmaceutical composition of embodiment 46, wherein the homodimer comprising the recombinant β-hexosaminidase variant α subunit is formulated at a concentration of about 20 mg/ml.

Embodiment 49 comprises a method of ameliorating the symptoms or slowing progression of TSD in a subject having TSD comprising administering to the subject an effective amount of the pharmaceutical composition of any one of embodiments 45-48.

Embodiment 50 comprises a method of ameliorating the symptoms or slowing progression of SD in a subject having SD comprising administering to the subject an effective amount of the pharmaceutical composition of any one of embodiments 45-48.

Embodiment 51 comprises the method of embodiment 49 or 50, wherein the pharmaceutical composition is administered to the subject intracerebroventricularly.

Embodiment 52 comprises the method of any one of embodiments 49-51, wherein the pharmaceutical composition is administered to the subject intracerebroventricularly weekly.

Embodiment 53 comprises the method of any one of embodiments 49-51, wherein the pharmaceutical composition is administered every other week.

Embodiment 54 comprises the method of any one of embodiments 49-51, wherein the pharmaceutical composition is administered at regular intervals, wherein the regular intervals are greater than every other week.

Embodiment 55 comprises a nucleic acid encoding the homodimer comprising the recombinant β-hexosaminidase variant α subunit of embodiment 44.

Embodiment 56 comprises a vector comprising the nucleic acid of embodiment 55 and one or more gene regulatory regions.

Embodiment 57 comprises the vector of embodiment 56, further comprising adeno-associated virus (AAV) inverted terminal repeats (ITR) at both the 5′ and 3′ end of the vector.

Embodiment 58 comprises an AAV viral particle comprising the vector of embodiment 57.

Embodiment 59 comprises a method of ameliorating the symptoms or slowing disease progression of TSD in a subject having TSD comprising administering an effective amount of the AAV viral particle of embodiment 58 to the subject having TSD.

Embodiment 60 comprises a method of ameliorating the symptoms or slowing disease progression of SD in a subject having TSD comprising administering an effective amount of the AAV viral particle of embodiment 58 to the subject having SD.

Embodiment 61 comprises a method of reducing GM2 ganglioside accumulation in a subject having TSD or SD comprising administering an effective amount of the pharmaceutical composition of any one of embodiments 45-48 to a subject having TSD or SD.

Embodiment 62 comprises a method of reducing the level of a biomarker of disease progression of TSD in a subject having TSD comprising administering an effective amount of the pharmaceutical composition of any one of embodiments 45-48 to a subject having TSD.

Embodiment 63 comprises a method of reducing the level of a biomarker of disease progression of TSD in a subject having TSD comprising administering an effective amount of the AAV viral particle of embodiment 58 to a subject having TSD.

Embodiment 64 comprises a method of reducing the level of a biomarker of disease progression of SD in a subject having SD comprising administering an effective amount of the pharmaceutical composition of any one of embodiments 45-48 to a subject having or SD.

Embodiment 65 comprises a method of reducing the level of a biomarker of disease progression of SD in a subject having SD comprising administering an effective amount of the viral particle of embodiment 58 to a subject having or SD.

Embodiment 66 comprises the method of any one of embodiments 62-65, wherein the biomarker of disease progression of TSD or SD is a GM2 and GA2, A2G0′ containing beta-linked terminal N-acetyl-D-hexosamine, BMP(22:6) phospholipid (Bis[monoacylglycero]phosphate)) or neurofilament light chain or a free glycan.

Embodiment 67 comprises the method of embodiment 49, wherein the subject is pre-symptomatic for TSD.

Embodiment 68 comprises the method of embodiment 49 or embodiment 67, wherein the subject has a TSD-associated mutation.

Embodiment 69 comprises the method of any one of claims 49 or 67-68, wherein the subject has an elevated level of a biomarker of TSD relative to a control level of the biomarker of TSD.

Embodiment 70 comprises the method of embodiment 69, wherein the elevated level of a biomarker of TSD is a measurement of the biomarker of TSD obtained from a sample from the subject.

Embodiment 71 comprises the method of embodiment 69 or embodiment 70, wherein the control level of the biomarker of TSD is a measurement of the biomarker of TSD from a sample from a subject that does not have a TSD-associated mutation.

Embodiment 72 comprises the method of embodiment 70 or embodiment 71, wherein the sample from the subject and the sample from the subject that does not have a TSD-associated mutation are quantified using liquid chromatography and mass spectrometry.

Embodiment 73 comprises the method of any one of embodiments 70-72, wherein the sample from the subject and the sample from the subject that does not have a TSD-associated mutation are obtained from the CSF or blood.

Embodiment 74 comprises the method of any one of embodiments 70-72, wherein the sample from the subject and the sample from the subject that does not have a TSD-associated mutation are blood plasma samples.

Embodiment 75 comprises the method of any one of embodiments 69-74, wherein the elevated level of the biomarker of the TSD is elevated by at least 100% to 4000% relative to the control level of the biomarker of TSD.

Embodiment 76 comprises the method of any one of embodiments 69-74, wherein the elevated level of the biomarker of the TSD is elevated by at least 2× to 100× relative to the control level of the biomarker of TSD.

Embodiment 77 comprises the method of any one of embodiments 69-76, wherein the biomarker is GM2, GA2, A2G0′ containing beta-linked terminal N-acetyl-D-hexosamine, BMP (22:6) phospholipid (Bis[monoacylglycero]phosphate)), neurofilament light chain or a free glycan.

Embodiment 78 comprises the method of embodiment 50, wherein the subject is pre-symptomatic for SD.

Embodiment 79 comprises the method of embodiment 50 or embodiment 78, wherein the subject has a SD-associated mutation.

Embodiment 80 comprises the method of any one of embodiments 50 or 78-79, wherein the subject has an elevated level of a biomarker of SD relative to a control level of the biomarker of SD.

Embodiment 81 comprises the method of embodiment 80, wherein the elevated level of a biomarker of SD is a measurement of the biomarker of SD obtained from a sample from the subject.

Embodiment 82 comprises the method of embodiment 80 or embodiment 81, wherein the control level of the biomarker of SD is a measurement of the biomarker of SD from a sample from a subject that does not have a SD-associated mutation.

Embodiment 83 comprises the method of embodiment 81 or embodiment 82, wherein the sample from the subject and the sample from the subject that does not have a SD-associated mutation are quantified using liquid chromatography and mass spectrometry.

Embodiment 84 comprises the method of any one of embodiments 81-83, wherein the sample from the subject and the sample from the subject that does not have a SD-associated mutation are obtained from the CSF or blood.

Embodiment 85 comprises the method of any one of embodiments 81-83, wherein the sample from the subject and the sample from the subject that does not have a SD-associated mutation are blood plasma samples.

Embodiment 86 comprises the method of any one of embodiments 80-84, wherein the elevated level of the biomarker of the SD is elevated by at least 100% to 4000% relative to the control level of the biomarker of SD.

Embodiment 87 comprises the method of any one of embodiments 80-84, wherein the elevated level of the biomarker of the SD is elevated by at least 2× to 100× relative to the control level of the biomarker of SD.

Embodiment 88 comprises the method of any one of embodiments 80-87, wherein the biomarker is GM2, GA2, A2G0′ containing beta-linked terminal N-acetyl-D-hexosamine, BMP (22:6) phospholipid (Bis[monoacylglycero]phosphate)), neurofilament light chain or a free glycan.

### EXAMPLES

### Example 1: Expression and Purification of Variant Hexosaminidase Enzymes

Expression constructs of variant hexosaminidase enzymes (HexD3, HexM, and Mod2B) were generated in the expression vector pXC17.4. The HexD3 construct was generated from DNA encoding the carboxy-portion of HexM ligated to DNA encoding the amino terminal portion (including signal sequence) of HexB. The various hexosaminidase encoding fragments were inserted into the expression vector pXC17.4.

The hexosaminidase encoding plasmids were transfected into suspension GSKO CHO (glutamine synthetase knock-out Chinese hamster ovary) cells. The cells were grown in CDCHO media with 6 mM glutamine in shake flasks at 37° C. and 8% CO₂. 40 μg of linearized plasmid DNA in 12×E6 cells was transfected using electroporation. The cells were plated at 2500 cells/well in CDCHO media (−)glutamine after transfection. The plates were incubated at 37° C. and 8% CO₂ for approximately 4-6 weeks to identify clonal growth. The colonies were then screened by an 4-MU activity assay for hexosaminidase enzyme activity and the colonies having the highest enzyme activity were transferred to 24 well plates in CDCHO media (−) glutamine, followed by a subsequent passage of the clones displaying the highest activity to 6 well plates. Ultimately, the clones were transferred to shake flasks to identify the best expressing clones in suspension culture.

Recombinant enzyme purification was carried out using standard protein purification techniques. Starting material was mammalian cell culture supernatant, from flask cultures described above, which was thawed from storage at −80° C. The material was adjusted with NaCl to reach a final concentration of 1 M, followed by 0.2 μm sterile filtration.

The filtered material was loaded onto a butyl hydrophobic interaction column, pre-equilibrated with butyl load buffer (20 mM Tris, 1 M NaCl, pH 7.5). The bound materials were eluted with a linear gradient over 10 column volumes, using butyl elution buffer (20 mM Tris, pH 7.5). Samples from the elution peaks were pooled, buffer exchanged into 20 mM Tris, pH 7.5, and loaded onto a Q anion exchange column. Bound proteins were then eluted with a linear gradient (10 column volumes) using Q elution buffer (20 mM Tris, 1 M NaCl, pH 7.5). Purified samples were then buffer exchanged using centrifugal spin concentrators and sterile-filtered for storage.

### Construction, Expression, Production, Purification and Formulation of Hexosaminidase Variants.

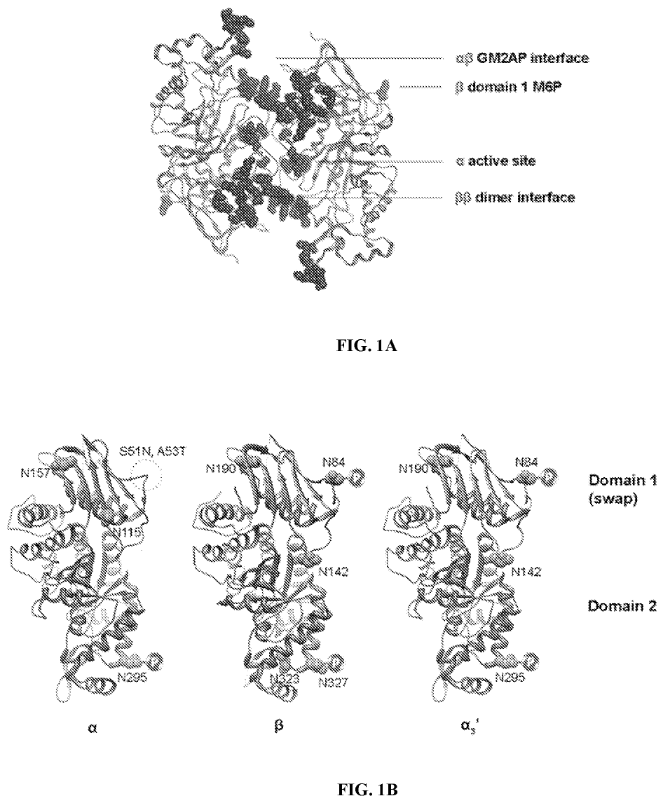

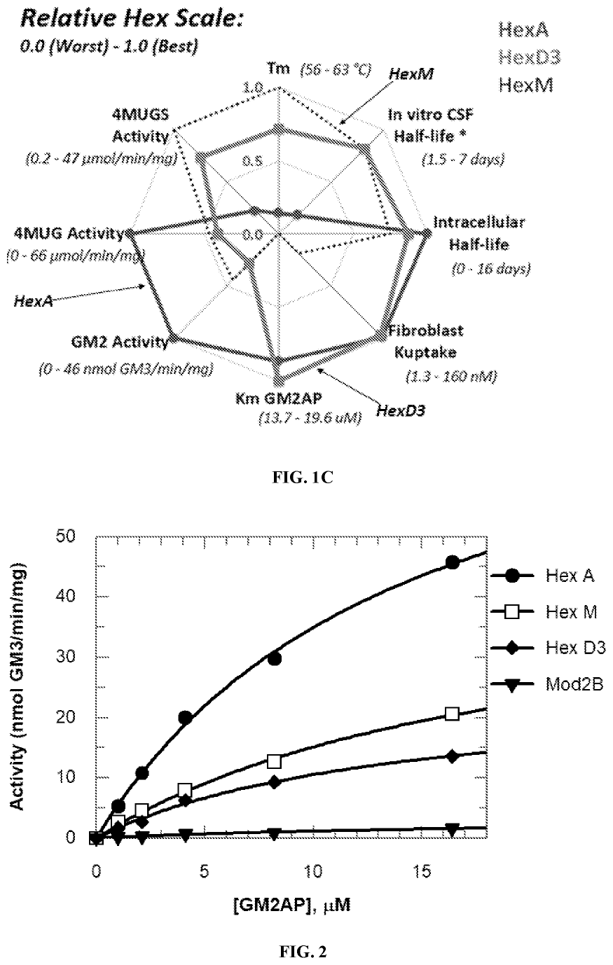

A DNA construct encoding HexD3 was generated using the DNA2.0 software package. HexD3 is a hybrid comprising sequences taken from both the α and the β subunits of β-hexosaminidase that builds on the structure of HexM. In particular, HexD3 contains the signal sequence and amino terminal sequences of the 0-subunit, which contains consensus glycosylation sites that increase the number of M6P groups on the variant enzyme, which is fused to the carboxy terminal of the α subunit that has substitutions or deletions at amino acid positions corresponding to S184K, P209Q, N228S, P229Δ, V230L, T231S, P429Q, K432R, D433K, 1436K, N466A, S491R, L493M, T494D, F495D, E498D, L508V, Q513V, N518Y, V519A, F521Y, and E523N of the native β-hexosaminidase α subunit sequence (FIG. 1B).

HexD3 retains the α/β interface for GM2AP binding, the β/β dimer interface for homodimer stability as well as the α subunit active site for HexM. HexD3 contains additional mannose-6-phosphorylation (M6P) sites, as compared to HexM, to increase receptor-mediated extracellular uptake and delivery to lysosomes. To add M6P sites, the entirety of domain 1 on the HexM u subunit scaffold was replaced with β-subunit domain 1. This replacement resulted in an increased M6P type N-glycan content (4/8) compared with HexM (2/6) and HexA (3/7).

HexM is a homodimeric variant hexosaminidase of the α subunit that has substitutions or deletions at amino acid positions corresponding to S184K, P209Q, N228S, P229Δ, V230L, T231S, P429Q, K432R, D433K, 1436K, N466A, S491R, L493M, T494D, F495D, E498D, L508V, Q513V, N518Y, V519A, F521Y, and E523N of the native β-hexosaminidase α subunit sequence.

For larger scale production, cells expressing variant hexosaminidase protein were grown in a bioreactor, in typical fed-batch production runs (14 days). The HexD3 protein was purified from the culture medium as follows. The harvested conditioned culture media was salt-adjusted to 1 M NaCl, then loaded onto a Butyl Sepharose 4 FF column. The variant hexosaminidase proteins were eluted by a decreasing salt gradient from the Butyl Sepharose 4 FF column, collected and dialyzed, and then loaded onto a Q Sepharose HP column. The variant hexosaminidase proteins were salt-eluted from the Q Sepharose HP column, concentrated, and then further purified via preparative Sephacryl S300 size exclusion chromatography. Using this purification procedure, highly purified, enzymatically active variant hexosaminidase proteins were produced. The purified variant hexosaminidase proteins were formulated at 20 mg/mL in artificial cerebrospinal fluid (CSF) consisting of 1 mM sodium phosphate, 148 mM sodium chloride, 3 mM potassium chloride at pH 7.2. Endotoxin levels were in an acceptable range for ICV administration: 0.006 EU/mg for HexA and 0.004 EU/mg for HexD3, for example.

To determine whether the N-glycan profiles differ in the HexA variants, glycan profiles were assessed using PNGase F to cleave the asparagine linked (N-Linked) oligosaccharides from denatured proteins. Once cleaved, oligosaccharides were dried and derivatized by reductive amination with the fluorescent dye 8-aminopyrene-1,3,6-trisulfonic acid. The labeled oligosaccharides were applied to a Sephadex G10 spin column to remove excess dye. The purified oligosaccharides were then separated by capillary electrophoresis. Electrophoresis of samples was performed on the P/ACE MDQ CE using a 65 cm N-CAP coated capillary with a 50 μm I.D along with the kit supplied N-CAP. FIG. 16 demonstrates that HexD3 and HexM had different glycosylation patterns compared to HexA. In FIG. 16, BPM means bis-phosphorylated oligomannose-type glycan structure, MPM means mono-phosphorylated oligomannose-type glycan structure, and Man refers to an oligomannose-type glycan structure. Man-7, -8, or -9 refers to the repeating mannose monosaccharide units combined to form the glycan structure.

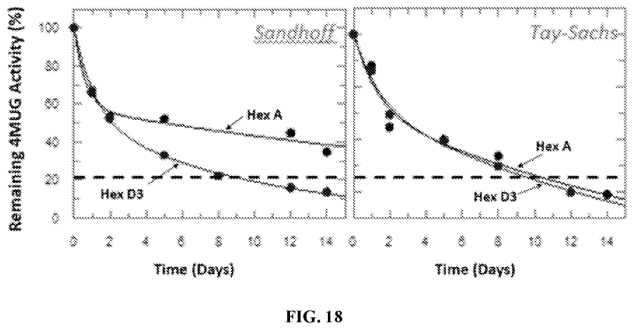

In addition, the cellular half-life of HexD3 and HexA was compared in FIG. 18. Cellular stability of HexD3 was comparable with the uptake of other lysosomal proteins (T_{1/2} 7-14 days). The cells were exposed to the Hex isozymes for 4 hours, washed, lysed and assayed for enzyme activity using the artificial substrate (MUGS). Each data point represents the average of 3 wells (n=3).

TABLE 3
Kinetic data of FIG. 18

| T_{1/2} (day) 2^{nd} | | |
|---|---|---|
| order rate of decay | Hex A | Hex D3 |
| Sandhoff k₁ | 0.4 | 0.8 |
| Sandhoff k₂ | 24 | 7 |
| Tay-Sachs k₁ | 0.9 | 1 |
| Tay-Sachs k₂ | 16 | 14 |

### Example 2: Generation of Hexa Enzyme

The hexosaminidase encoding plasmids (alpha subunit and beta-subunit) were co-transfected into suspension GSKO CHO cells using electroporation. The cells were plated at 2500 cells/well in CDCHO media (−) glutamine after transfection, followed by incubation at 37° C. and 8% CO₂ for approximately 4-6 weeks to identify clonal growth. The colonies were then screened by a 4-MU activity assay for hexosaminidase enzyme activity and western blot analysis using an anti-HexA antibody and an anti-HexB antibody, to determine expression of both subunits. The majority of screened colonies expressed only the beta subunit, and thus the HexB homodimer; however, a few colonies were found to stably express a mixture of the hexosaminidase isozymes, including a HexA heterodimer. The cells expressing the HexA heterodimer were identified and stored as a suspension culture.

Purification was carried out using standard protein purification techniques. In a standard purification method, stored conditioned culture media, was thawed from storage at −80° C. in a 37° C. water bath or at room temperature. The material was pH adjusted to pH 6.0 and 0.2 μm filtered.

The filtered material was directly loaded onto an anion exchange resin that had been pre-equilibrated with anion exchange load buffer (20 mM BisTris, pH 6.0). The bound material was then washed for 10 column volumes with a wash buffer (20 mM BisTris, 75 mM NaCl pH 6.0). Following the wash, the protein of interest was eluted out with 10 column volumes of the elution buffer (20 mM BisTris, 200 mM NaCl pH 6.0). The elution was collected and 5M NaCl was added to the sample until conductivity was matched with the second step phenyl hydrophobic interaction column equilibration buffer (20 mM NaPO₄, 1.5 M NaCl, pH 7.0). After pH adjustment to pH 7.0, the sample was loaded onto the column and bound proteins were first washed with awash buffer (20 mM NaPO₄, 1.3 M NaCl pH 7.0) for 10 column volumes then eluted with 10 column volumes of the elution buffer (20 mM NaPO₄ pH 7.0). Final pooled material was buffer exchanged into artificial CSF formulation buffer, concentrated and sterile filtered for storage at −80° C.

### Example 3: Natural Substrate Enzyme Activity Assay and Enzyme Thermostability

The enzymatic activity of the modified recombinant human Hex homodimers were determined in vitro in a natural substrate assay.

Materials used in the natural substrate assays included: Ganglioside Monosialylated-2 substrate (GM2, Phosphatidylcholine (PC), Phosphatidylinositol (PI) and Cholesterol (Chol)). The materials were used to prepare liposomes at a molar ratio of 10:50:20:20, respectively. Briefly, GM2 (1:1 in toluene:ethanol), PC (2:1 in toluene:ethanol), PI (in chloroform), and Cholesterol (2:1 in chloroform:methanol) were mixed together in the specified molar ratios, vacuum dried then resuspended in 1 mL of 1 mM Tris buffer, pH 7.4. The GM2 concentration in the final liposomal preparation was 1 mg/mL and was stored at −20° C. Modified recombinant human Hex proteins were concentrated to approximately 20 mg/mL, formulated in artificial CSF (0.2 mM NaH₂PO₄, 0.8 mM Na₂HPO₄, 3 mM KCl and 148 mM NaCl, pH 7.2) and stored at −80° C. Recombinant human GM2 activator protein (GM2AP) containing an N-terminal 6×-histidine tag was prepared at a concentration of 1.3 mg/mL in acidic PBS (pH 6.5) containing 10% glycerol and stored at −80° C.

Natural Substrate Assay—In polypropylene microcentrifuge tubes, 2× serial dilutions of GM2AP were prepared, from 1.26 mg/mL (66 μM) to 0.079 mg/mL (4 μM) plus one blank. In 6 separate polypropylene centrifuge tubes, 5 μL of each of the following assay components were mixed: assay buffer (0.2M Sodium Citrate, pH 4.4 containing 1 mg/mL BSA), GM2 liposomes and hexosaminidase (0.2 mg/mL). Final concentrations of each reaction component in the assay were calculated by dividing the starting concentration by 4, since equal volumes of each reaction component were added to the mix (5 μL/each, total reaction volume=20 μL).

To start the reaction, GM2AP dilutions (5 μL) were added to each tube, including a blank (no GM2AP), followed by incubation for 1 hour at 37° C. The 20 μL reaction was quenched by addition of 50 μL of Methanol. Samples were vortexed vigorously for 10 seconds followed by centrifugation at 12,000 rpm for 30 seconds. Supernatants were transferred to polypropylene HPLC vials to assess product formation (GM2 to GM3) by mass spectrometry.

HPLC—20 μL of the quenched supernatant was injected on a C4 RP column which had previously been equilibrated with 60% methanol, 5 mM Ammonium Formate and 1% Formic Acid (Mobile Phase A). GM2 and GM3 gangliosides were eluted using a 0-50% gradient over 10 minutes with 99% methanol, 5 mM Ammonium Formate and 1% Formic Acid (Mobile Phase B).

Mass Spectrometry—the amount of GM2 and GM3 in the final reaction mix was quantified by mass spectrometry using [M+2H]⁺² extracted ions for each ganglioside (m/z 701 and 600, respectively). Saturation was not observed under these conditions, so specific activity was calculated using the highest concentration of GM2AP and has units in nmol GM3 produced/min/mg of Hex (TABLE 4).

TABLE 4

| | Specific | | |
|---|---|---|---|
| | Activity | | |
| | GM2 | Vmax | |
| | (nmol | (nmol | Km |
| Protein | GM3/ | GM3/ | [GM2AP], |
| construct | min/mg) | min/mg) | μM |
| HexA (wild-type; α/β | 45.8 | 85.8 ± 5.4 | 14.5 ± 1.6 |
| heterodimer) | | | |
| HexM (Modified α/α | 20.6 | 44.8 ± 3.7 | 19.6 ± 2.5 |
| homodimer) | | | |
| HexD3 (Modified α/α | 13.6 | 25.0 ± 2.5 | 13.7 ± 2.4 |
| homodimer) | | | |
| Mod2B (Modified β/β | 1.6 | 3.5 ± 0.8 | 20.4 ± 7.3 |
| homodimer) | | | |

GM2AP-dependent kinetics for each Hex protein are shown in FIG. 2. Using this natural substrate assay, exemplary modified Hex homodimers, including HexD3, were shown to have enzymatic activity in vitro. The measured enzymatic activity toward the natural substrate (GM2) is 25-30% compared to that of the wild type Hex heterodimer, while Mod2B shows only trace activity under these conditions. GM2AP binding kinetics to the Hex homodimers was comparable to the wild type heterodimer, suggesting that the differences observed in the specific activity were due to the catalysis of the substrate and not the binding of GM2AP to the Hex homodimer.

TABLE 5
Kinetic data of FIG. 2

| | | Std. Error | | Std. Error |
|---|---|---|---|---|
| Construct | Vmax | of Vmax | Km | of Km |
| Hex A | 85.7638 | 5.4903 | 14.5356 | 1.6092 |
| Hex M | 44.8184 | 3.7100 | 19.5973 | 2.5583 |
| Hex D3 | 24.9866 | 2.4713 | 13.6721 | 2.3786 |
| Mod2B | 3.5240 | 0.8086 | 20.4290 | 7.2727 |

Thermostability or the melting temperature (Tm) of Hex variants was determined by nano differential scanning fluorimetry method using Prometheus NT.48. Protein samples for thermal unfolding experiments were prepared in 1 mM sodium phosphate, 148 mM sodium chloride, 3 mM potassium chloride, pH 7.2 at the final concentrations of 0.013-2.4 mg/ml. Fluorescence-based thermal unfolding at the increasing temperature (from 20° C. to 95° C. at 1° C./min) was monitored at emission wavelengths of 350 nm and 330 nm. The fluorescence measurements were used to obtain Tm values of wild-type and modified Hex proteins. Compared to less stable wild-type HexA (a/O) and HexS (α/α) dimers (Tm=56-57° C.), modified Hex homodimers exhibit increased thermal stability, indicated by a higher Tm range of 60-63° C. (TABLE 6).

TABLE 6

| Protein construct (0.013-2.4 mg/ml) | Tm (° C.) |
|---|---|
| HexA (wild-type; α/β heterodimer) | 56.5 ± 0.8 |
| HexB (wild-type; β/β homodimer) | 62.2 ± 0.8 |
| HexS (wild-type; α/α homodimer) | 55.8 ± 0.4 |
| HexM (Modified α/α homodimer) | 62.1 ± 0.1 |
| HexD3 (Modified α/α homodimer) | 59.8 ± 0.4 |
| Mod2B (Modified β/β homodimer) | 62.8 ± 1.1 |

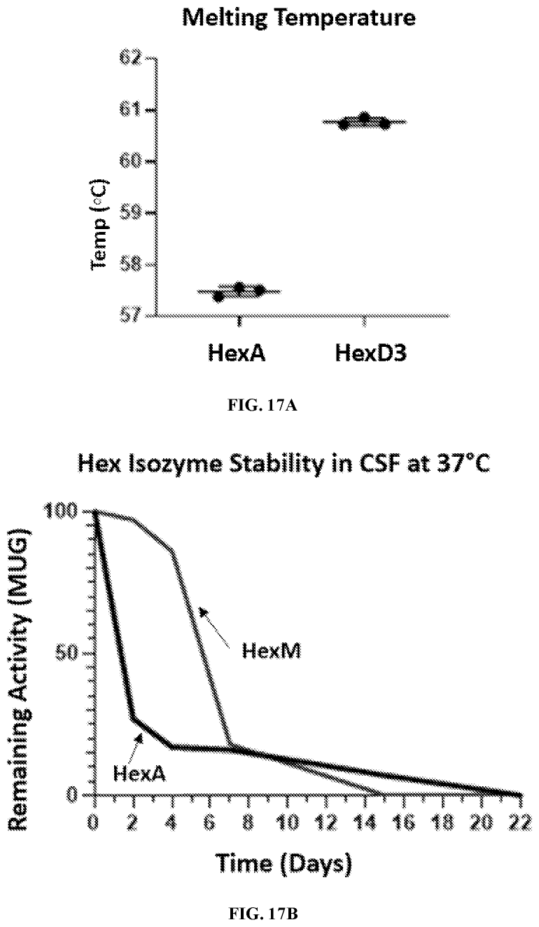

Thermal stability was consistent among different lots of HexA and HexD3 with HexD3 being more stable then HexA, as shown in FIG. 17A. FIG. 17A shows the melting temperature of 3 measurements of melting temperature (Tm) for HexA and HexD3. To determine thermal stability, differential scanning calorimetry (DSC) was performed. HexA and HexD3 protein were prepared at 25 mg/ml in 1 mM PO₄, 4% Trehalose, 0.005% PS20, 150 mM NaCl at pH 7.0. All experiments were conducted in the 20-100° C. temperature range at a heating rate (dT/dt) of 1° C. min⁻¹. For DSC, under an excess pressure of 3 atm, the change in thermal power (dQ/dt) as a function of temperature was automatically recorded by the TA Nano-DSC instrument and NanoAnalyze software. The raw data were further processed to yield the melting isotherms of excess heat capacity (Cp) as a function of temperature (T) using the following relationship: Cp=[(dQ/dt)]/[(dT/dt)PV], where P is the initial concentration of protein loaded into the calorimetric cell and V is the effective volume of calorimetric cell (0.3 ml). Stability in CSF measured by remaining MUG activity showing increased stability of HexM over HexA. MUG (4-methylumbelliferyl-2-acetamido-2-deoxy-β-D-glucopyranoside) and MUGS (4-methylumbelliferyl-2-acetamido-2-deoxy-β-D-glucopyranoside-6-sulfate) are synthetic fluorogenic substrates readily used to detect the activity of HexA and modified Hex proteins. HexD3 stability is predicted to be similar to HexM given they share the same backbone. Hex isozymes (1 μg/mL final) were spiked into human CSF containing 50 mM BisTris, pH 7.3 buffer. At designated time points, 10 uL of sample was assayed in the presence of 1 mM 4MUG for 30 minutes at 37° C. (FIG. 17B). FIG. 17B demonstrates that HexM, a modified α/α homodimer, maintains a higher MUG activity than HexA for as long as about 6 days, whereas HexA MUG activity sharply decreases after one day. Thus, HexD3 has been demonstrated to be more thermostable than HexA and is expected to retain MUG activity for a longer period of time compared to HexA (FIG. 17A and FIG. 17B).

### Example 4: Synthetic Substrate Enzyme Activity Assays

The enzymatic activity of the modified recombinant human Hex homodimers were determined in vitro in two synthetic substrate assays. MUG and MUGS are fluorogenic substrates readily used to detect the activity of HexA and modified Hex proteins. While MUG and MUGS can be metabolized by several isoenzymes, GM2 is particular to HexA.

MUGS and MUG were each prepared to a final concentration of 100 mM (stock solution) in DMSO and stored at −20° C. Stock solutions of 4-methylumbelliferone (4-MU Standard) were prepared at 10 mM in DMSO and stored at −20° C. in small aliquots. Modified recombinant human Hex proteins were concentrated to approximately 20 mg/mL, formulated in artificial CSF (0.2 mM NaH₂PO₄, 0.8 mMNa₂HPO₄, 3 mM KCl and 148 mMNaCl, pH 7.2) and stored at −80° C.

Synthetic Substrate Assay—On a black 96 well dilution plate, 2× serial dilutions of standards were used, from 100 μM to 1.56 μM plus one blank. On the same plate, protein samples diluted in assay buffer (56 mM Sodium Citric Acid, 88 mM Sodium Phosphate (dibasic), 0.5 mg/mL Bovine Serum Albumin, pH 4.4) were prepared in several dilutions to ensure they were within the standard curve.

50 μL of standards (100 μM to 1.56 μM) or protein samples were added to 50 μL of substrate (1 mM), followed by incubation for 30 minutes at 37° C. The reaction was quenched by addition of 200 μL of stop buffer (0.5 M Glycine/NaOH, pH 10.7). The plates were read at Ex355/Em460 with 455 cut off on a 96-well fluorescent plate reader.

Using this synthetic substrate assay, exemplary modified Hex homodimers, including HexD3, were shown to have enzymatic activity in vitro. The measured enzymatic activities toward the synthetic 4-MUG and 4-MUGS substrate were comparable to that of the wild type Hex heterodimer (TABLE 7).

HexD3 has differences in activity and stability compared to HexM and HexA (TABLE 7). An improvement in affinity to GM2AP and some retained activity towards the artificial substrates 4-MUG and 4-MUGS were observed for HexD3 compared to HexM. The domain 1 modification weakened the HexD3-GM2 specific activity. However, as a result of the enhanced M6P content, the Kᵤₚₜₐₖₑ for HexD3 is better than HexM and matches that of HexA. Furthermore, HexD3 showed greater thermostability than HexA. Of note, the β-glycosylation site N142 is six amino acids closer to the N-terminus than the regional α subunit counterpart N115, which may affect stability. Similar to HexA, HexD3 was found to be functional in both SD and Tay Sachs human fibroblast cells.

TABLE 7

| | Specific | Specific | |
|---|---|---|---|
| | Activity | Activity | |
| Protein | (MUG; | (MUGS; | MUG/MUGS |
| construct | μmol/min/mg) | μmol/min/mg) | ratio |
| HexA (wild-type; α/β | 66 | 23 | 2.9 |
| heterodimer) | | | |
| HexB (wild-type; β/β | 79 | n.d. | N/A |
| homodimer) | | | |
| HexS (wild-type; α/α | 19 | 23 | 0.80 |
| homodimer) | | | |
| HexM (Modified α/α | 31 | 47 | 0.66 |
| homodimer) | | | |
| HexD3 (Modified α/α | 27 | 39 | 0.69 |
| homodimer) | | | |
| Mod2B (Modified β/β | 20 | 16 | 1.25 |
| homodimer) | | | |

### Example 5: β-Hexosaminidase Cellular Uptake Assay

The ability of variant β-Hexosaminidase enzymes to enter cells via receptor-mediated endocytosis was determined using a cellular uptake assay. The cell uptake assay described here measured enzyme uptake by the cation independent mannose 6-phosphate receptor (CI-MPR) in human fibroblasts from SD patients. Mannose 6-phosphate (M6P) was used as a competitive inhibitor to demonstrate uptake via the CI-MPR receptor. Data was collected to generate a saturation curve for enzyme uptake and determine the kinetic parameter, Kᵤₚₜₐₖₑ, of the process.

Prior to the uptake assay (24 hours) a confluent T-75 flask of human SD fibroblasts (GM00203) was plated at a density of 1×10⁵ cells per 0.5 mL per well in 24-well plates. Enzyme samples at 0.4-400 nM were prepared in uptake media: 496.6 mL DMEM+13.0 mL 1.0 M HEPES+5.2 mL 50.0 mg/mL BSA+5.2 mL 200.0 mM L-Glutamine. For uptake inhibition, 5.0 mM M6P was added to appropriate samples.

Growth media (445 mL DMEM+50.0 mL FBS+5.0 mL 20.0 mM L-Glutamine) was aspirated from cells and replaced by 0.5 mL of uptake media per well. Following a 4-hour incubation, plates were washed 2 times with 1 mL Dulbecco's PBS. 120.0 μL of M-PER lysis buffer was added to the plates and agitated with a reciprocating rocker at room temperature for 10 minutes and 150 motions/min². Lysate was stored at −80° C. until ready to assay.

For the enzyme assay, 10 μL of each lysate was added in duplicate to the black 96-well plate (VWR).

For each enzyme load, uptake was expressed as nmoles of 4-MU liberated in 30 minutes. For saturation curves, enzyme concentrations ranging from 0.4-400 nM were used to generate a saturation curve using the assays described above.

Cellular stability of the modified β-Hex proteins was determined by monitoring intracellular 1-Hex activity over the period of 14 days. Confluent human SD fibroblasts in 12-well plates were treated with β-Hex at 400 nM final concentration for 24 hours in a 5% atmosphere of CO₂ at 37° C. After the 24-hour incubation, cells were switched to growth media in the absence of β-Hex. For each time point (1 day, 2 days, 5 days, 8 days, 12 days, and 14 days), cells were lysed in 200 μL of M-PER lysis buffer added to the plates and agitated with a reciprocating rocker at room temperature for 10 minutes and 150 motions/min². Cell lysates were assayed for β-Hex activity using a fluorogenic 4-MU substrate. Reduction of β-Hex activity over the 14-day sample period were fit to second-order kinetics to approximate a cellular half-life of the protein.

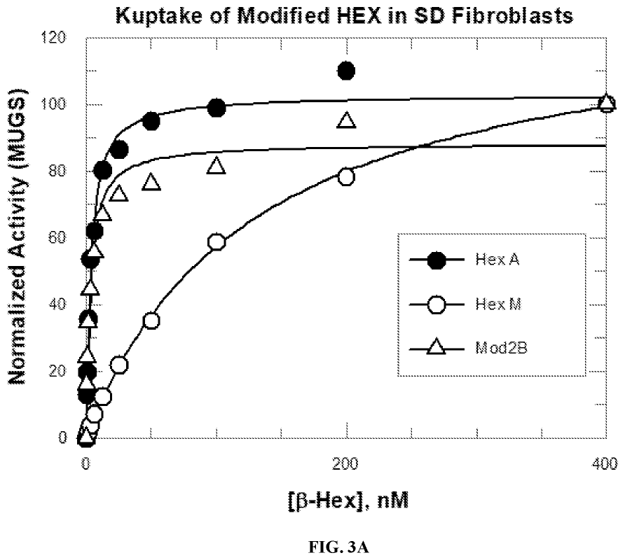

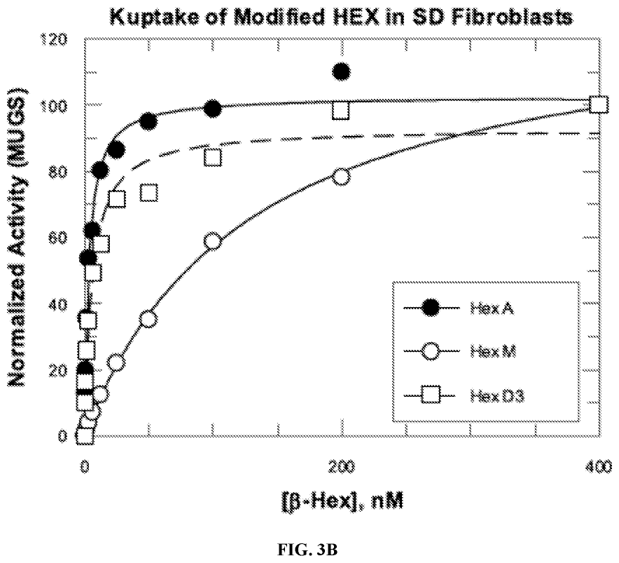

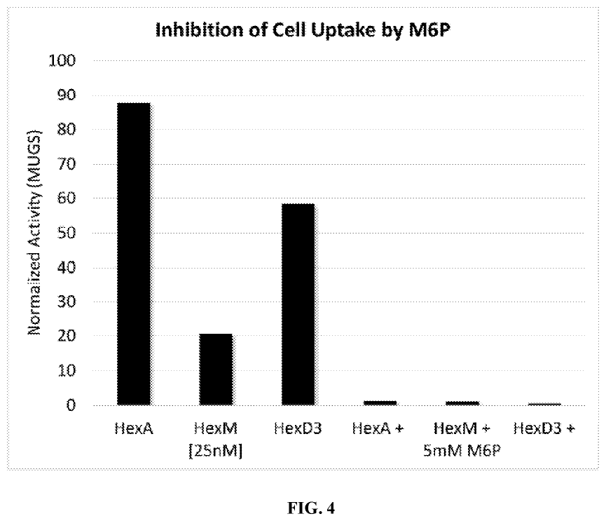

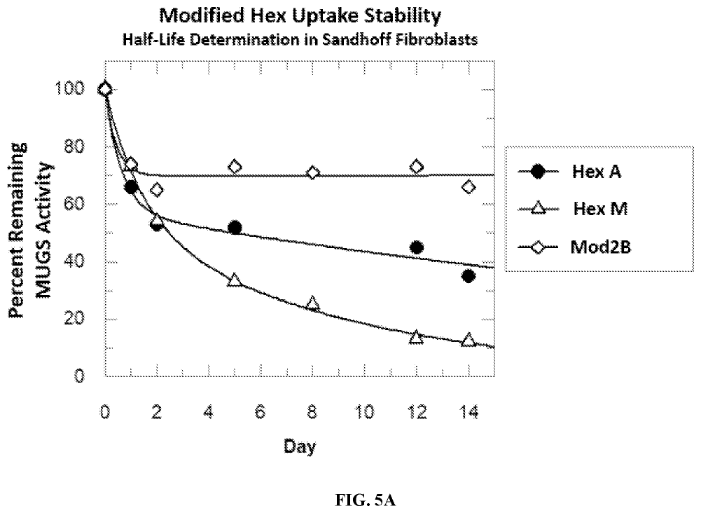

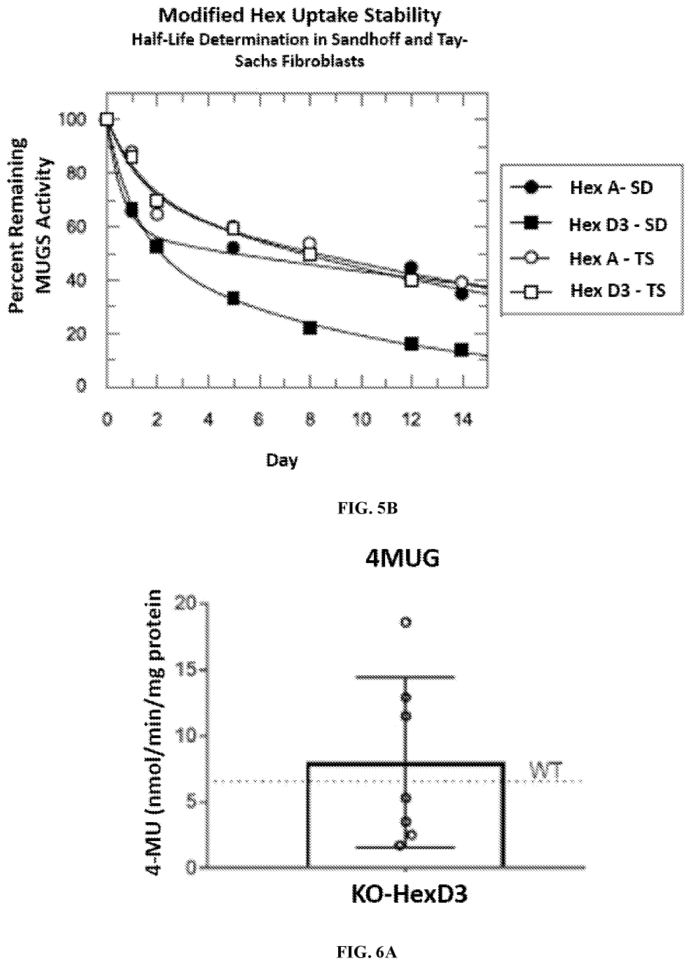

Using this assay, β-Hex variants, including wild type α/β heterodimer (HexA), modified β/β homodimer (Mod2B), and modified c/a homodimer (HexD3) were shown to be efficiently internalized into SD fibroblasts with average Kᵤₚₜₐₖₑ range of 3 to 13 nM (FIGS. 3A and 3B). In contrast, modified α/α homodimer (HexM), exhibited poor uptake efficiency under these experimental conditions (>120 nM) (FIGS. 3A and 3B). Uptake of HexM, HexA and HexD3 was inhibited by M6P, indicating cellular uptake proceeds via the CI-MPR (FIG. 4). Cellular stability of the 1-Hex variants was on par with other lysosomal enzymes, with an observed half-life of ˜6-9 days (FIGS. 5A and 5B). Biphasic decay may reflect assay conditions (i. e. high initial dose of 400 nM for 24 hours) as indicated by the calculated different rate constants of the respective enzymes. The late phase of decay curve demonstrated typical lysosomal stability with an estimated half-life of ˜6-14 days for modified homodimers and ˜16-25 days for the wild-type heterodimer as indicated by the T_{1/2 (Late)} for HexA in SD or TS.

TABLE 8
Kinetics of FIG. 3A

| | | Std. Error | | Std. Error |
|---|---|---|---|---|
| Construct | Vmax | of Vmax | Km | of Km |
| Hex A | 102.7180 | 2.0158 | 3.3085 | 0.3236 |
| Hex M | 131.1395 | 2.3074 | 128.6621 | 5.3970 |
| Mod2B | 88.1564 | 3.3611 | 3.0053 | 0.5756 |

TABLE 9
Kinetics of FIG. 3B

| | | Std. Error | | Std. Error |
|---|---|---|---|---|
| Construct | Vmax | of Vmax | Km | of Km |
| Hex A | 102.7180 | 2.0158 | 3.3085 | 0.3236 |
| Hex M | 131.1395 | 2.3074 | 128.6621 | 5.3970 |
| Hex D3 | 92.9713 | 3.4393 | 5.7839 | 0.9793 |

TABLE 10
Kinetics of FIG. 5A

| Construct | Hex A | Hex M | Mod2B |
|---|---|---|---|
| Initial value 1 | 42.7999 | 44.0250 | 30.4666 |
| Initial value 1 | 8.5996 | 9.1760 | 6.9092 |
| std. error | | | |
| Rate constant 1 | 1.5584 | 0.7223 | 2.2618 |
| Rate constant 1 | 0.8964 | 0.1860 | 1.5735 |
| std. error | | | |
| Initial value 2 | 57.3239 | 56.2869 | 69.6230 |
| Initial value 2 | 7.2619 | 9.1852 | 5.2035 |
| std. error | | | |
| Rate constant 2 | 0.0276 | 0.1121 | −0.0003 |
| Rate constant 2 | 0.0132 | 0.0180 | 0.0077 |
| std. error | | | |
| T_{1/2 (initial)} | 0.4 days | 1 day | 0.3 days |
| T_{1/2 (late)} | 25 days | 6 days | stable |

TABLE 11
Kinetics of FIG. 5B

| Construct | Hex A - SD | Hex A - TS | Hex D3 - SD | Hex D3 - TS |
|---|---|---|---|---|
| Initial value 1 | 42.5716 | 29.6571 | 46.5641 | 27.5500 |
| Initial value 1 | 8.3938 | 15.2082 | 4.3709 | 11.9428 |
| std. error | | | | |
| Rate constant 1 | 1.6050 | 0.7909 | 0.8707 | 0.7160 |
| Rate constant 1 | 0.8576 | 0.7295 | 0.1198 | 0.4663 |
| std. error | | | | |
| Initial value 2 | 57.5527 | 71.5947 | 53.2891 | 73.1086 |
| Initial value 2 | 7.1018 | 14.5027 | 4.3061 | 11.8163 |
| std. error | | | | |
| Rate constant 2 | 0.0286 | 0.0439 | 0.1025 | 0.0498 |
| Rate constant 2 | 0.0129 | 0.0205 | 0.0094 | 0.0182 |
| std. error | | | | |
| T_{1/2 (initial)} | 0.4 days | 0.9 days | 0.8 days | 1 day |
| T_{1/2 (late)} | 24 days | 16 days | 7 days | 14 days |

### Example 6: In Vivo Hexd3 Enzyme Activity Following Intracerebroventricular (Icv) Administration

To determine the activity of recombinant HexD3 in vivo, HexD3 was administered to a β-hexosaminidase knockout (HEXB^{-/-} KO) mouse model for SD by intracerebroventricular (ICV) injection. A permanent cannula was implanted in the mouse at 6 weeks of age. After 1 week of recovery and starting at 7-9 weeks of age, HexD3 was injected as a bolus volume of 5 μL, administered over a period of 5-10 minutes. ICV injections were given once per week over a two-week period (100 μg/injection) for a total of 3 injections. Animals were euthanized 24 hrs after the 3rd ICV injection by cardiac puncture followed by perfusion with PBS. Brains were isolated and flash frozen. Endpoint measurements included the detection and distribution of HexD3 in the brain, tissue activity levels of enzyme, correction of cellular lysosome using LAMP2 signal, lysosomal biomarker BMP, and reduction in GM2 ganglioside storage material.

The enzymatic activity of HexD3 proteins in vivo was determined using a fluorescent labeled synthetic substrate activity assay as described below.

Frozen tissue samples (brain hemispheres weighing 150-300 mg each) were transferred to homogenization tubes preloaded with zirconium oxide beads and 600 μL of cold HPLC grade water was added to each sample. Samples were homogenized with a Bullet Blender in a cold room set to 4° C. An aliquot (300 μL) of this homogenate was removed for Sensi-Pro assay, which measures substrate accumulation, and mass spectrometry analysis. The remaining tissue homogenate in the lysing tubes was homogenized again after addition of 420 μL of chilled T-Per buffer with protease inhibitor cocktail. This homogenate was transferred to anew tube containing an additional 300 μL of T-Per with protease inhibitors. The T-Per homogenate samples were centrifuged in a refrigerated table-top centrifuge for 15 minutes at maximum speed (14,000 rpm) and the supernatant was transferred to anew tube and used for HexD3 activity assays. These prepared samples are subjected to enzyme activity analysis as described below.

### Assays of HexD3 Enzyme Activity In Vivo

4-MUG and 4-MUGS were each prepared to 100 mM concentration in DMSO and stored at −20° C. Stock solution of 4-methylumbelliferone (4-MU standard), prepared at 10 mM in DMSO and stored at −20° C. in small aliquots.

On a clear 96 well dilution plate (3× serial dilutions of 4-MU standards were prepared from 75 μM to 0.1 μM plus one blank and 3× serial dilutions of HexD3 standards were prepared from 4 ng/well to 0.0055 ng/well plus 6 ng/well and one blank. Both standards were prepared in brain protein homogenate from untreated SD mice used as background matrix. Samples are prepared at a protein concentration of 0.04 μg/L.

To measure HexD3 activity in tissues, 50 μL of all standards and working samples as described above were transferred in duplicate to a black non-treated polystyrene 96 well plate. For each assay 50 μL of substrate at 1 mM in assay buffer (citrate phosphate, pH 4.4) was added to each well, followed by incubation for 30 minutes at 37° C. The reaction was then quenched by addition of 200 μL of stop buffer (0.5 M glycine/0.3 M NaOH, pH 10.3) to each well. Fluorescence was measured on a FlexStation 3 multi-mode microplate reader with excitation wavelength at 365 nm and emission wavelength at 440 nm and a cutoff of 435 nm.

Raw data were acquired using SoftMax Pro 6.3. The activity levels of the samples were calculated by extrapolating the amount of product (4-MU) generated in the reaction from a 7-point standard curve that was prepared by spiking a known amount of 4-MU into SD mouse brain protein extract used as background matrix. Activity levels were expressed as nmol 4-MU cleaved/mg protein/minute. The amount of active human HexD3 in tissue homogenate samples from ICV treated SD mice was back-calculated from a standard curve that was prepared by spiking a known amount of recombinant human HexD3 into SD mouse brain protein extract used as background matrix.

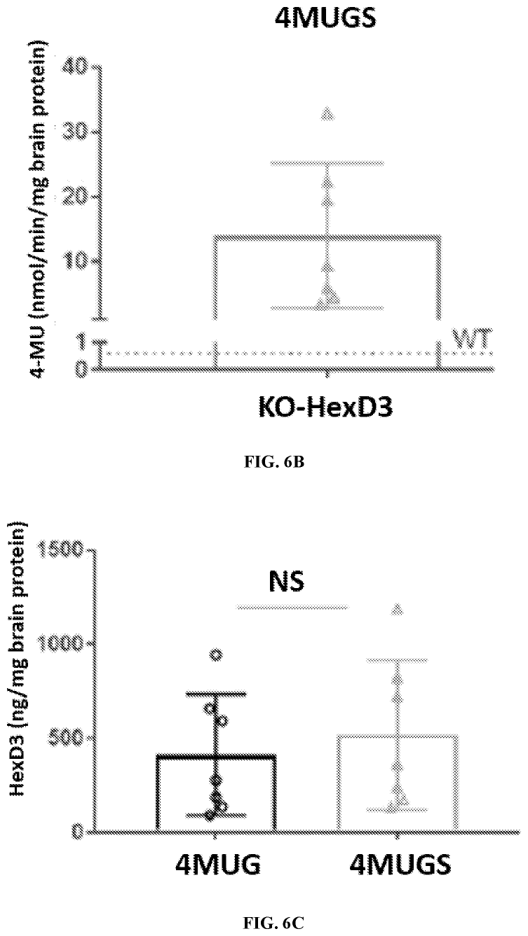

Using this assay, HexD3 activity toward the synthetic fluorogenic 4-MUG and 4-MUGS substrates was detected in brain homogenate samples from SD mice treated with HexD3 while no activity was detected in samples from untreated SD mice. The average activity measured in brain homogenates from mice dosed with HexD3 was comparable to the hexosaminidase activity measured in brain homogenates from wild-type mice (FIGS. 6A and 6B). No activity towards either substrate was detected in brain homogenates from untreated SD mice (FIG. 6B). The amount of HexD3 per mg brain protein that was determined based on the activity assays was similar regardless of the substrate that was used, which correlated well with the mass spectrometry data (FIG. 6C). Based on specific activity, similar amounts were calculated regardless of the fluorogenic substrate used (NS, not significant, t-test).

### Example 7: Detection and Quantification of Hexd3 Enzyme in Mouse Brain Homogenate

The amount of HexD3 protein in wild type or SD mouse brain protein extract was determined using liquid chromatography (LC)-Parallel Reaction Monitoring (PRM)-based targeted mass spectrometry (MS).

### Sample Preparation

Frozen mouse brain samples were transferred to homogenization tubes preloaded with zirconium oxide beads, and 600 μL of cold HPLC grade water was added to each sample. Samples were homogenized with a Bullet Blender in a cold room (4° C.). Subsequently four parts of Biognosys lysis buffer was added to one part of brain water homogenate. The protein concentration of all samples was determined using a BCA assay. Samples were reduced and alkylated using Biognosys' reduction and alkylation solution with 2-chloroacetamide and digested overnight with sequencing grade modified Trypsin at a protein:protease ratio of 50:1. The digested peptides were cleaned up for mass spectrometry using C18 MacroSpin columns. The cleaned-up peptides were dried down using a SpeedVac system, and re-dissolved in LC solvent A (1% acetonitrile in water with 0.1% formic acid (FA)) containing iRT-peptide mix (Biognosys) for retention time calibration. Peptide concentration was measured at 280 nm with SPECTROstar® Nano spectrophotometer. The following custom stable isotope-labeled reference peptides were designed specifically for HexD3 detection in the background of other brain proteins: GSYSLSHIYTAQDVK (SEQ ID NO: 15), IQPDTIIQVWR (SEQ ID NO: 16), ISYGQDWR (SEQ ID NO: 17). For the absolute quantification of HexD3 protein, the equimolar pool of the peptides was used and spiked into the final peptide samples at known concentration as internal standards.

### LC-PRM Measurements

For the quantification of HexD3, peptides (1 μg per sample) were injected to an in-house packed C18 column (ReproSil-Pur120C18AQ, 1.9 μm, 120 Å pore size; 75 μm inner diameter, 50 cm length) on a Thermo Scientific Easy nLC 1200 nano-liquid chromatography system. LC solvents were A:1% acetonitrile in water with 0.1% FA; B: 15% water in acetonitrile with 0.1% FA. The LC gradient was 5-40% solvent B in 50 minutes followed by 40-90% B in 2 minutes and 90% B for 12 minutes (total gradient length was 64 minutes). LC-PRM runs for peptide quantification were carried out on a Thermo Scientific Q Exactive mass spectrometer equipped with a standard nano-electrospray source. Collision energies were 25 eV according to the vendor's specifications.

An unscheduled run in PRM mode was performed before data acquisition for retention time calibration using Biognosys' iRT concept. The acquisition window was 4 minutes. Signal processing and data analysis were carried out using SpectroDive™ 7.0. A Q-value filter of 1% was applied. The absolute quantification was determined by comparing the abundance of the known internal standard peptides with the endogenous peptides. The ratio of the areas under the curve (between the endogenous and reference peptide) was used to determine the absolute levels of HexD3 in the samples.

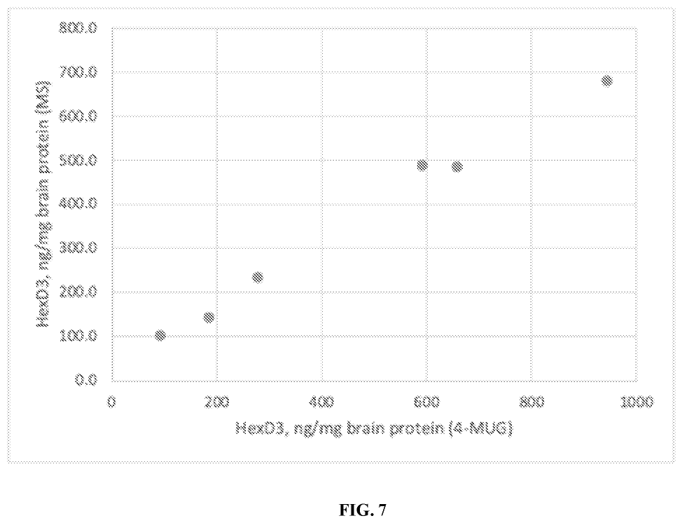

Using this assay, HexD3 proteins were detected and quantified in mouse brain protein extract. The measured HexD3 quantity correlated (r=0.995) with quantities of HexD3 measured using the 4-MUG/4-MUGS based activity assay (FIG. 7).

### Example 8: In Vivo Hexd3 Distribution and Lysosomal Correction

Immunohistochemical (IHC) staining and in situ enzymatic activity were used to assess the distribution of HexD3 in mouse brain after ICV administration and to assess the ability of HexD3 ICV ERT to correct the lysosomal defect in an animal model system.

Brain tissues were immersion fixed in formalin and embedded in paraffin. For a majority of the IHC analyses, 7 μm thick sagittal sections were taken at approximately the superior sagittal sinus (sagittal midline) delineating the left and right brain hemispheres. Region matching for the sagittal sections was conducted utilizing the lateral ventricle, hippocampus, thalamus, pons (pontine reticular nucleus), and the VIa lobule of the central cerebellar region, corresponding to position 164 of the mouse Allen Brain Atlas. Sections were immunostained with antibodies against LAMP2. For antigen retrieval, slides were immersed in Discovery CC1 solution for 30 minutes at 95° C. The blocking buffer consisted of a 2% NDS, 0.10% BSA and 0.3% Triton solution in 1×TBS. Donkey anti-rat IgG (H+L) highly cross-adsorbed secondary antibody conjugated to Alexa Fluor 488 was used to detect anti-LAMP2 antibodies. Slides were mounted in DAPI Fluoromount to visualize cellular nuclei.

Signal isolation and analysis was conducted via Adobe Photoshop and Image J software packages. For quantification of fluorescence signal, the % area of the total adjusted signal was utilized for downstream analysis. ANOVA with Tukey post-hoc testing was utilized to analyze the variance between each of the treatment groups. P<0.05 was determined to be statistically significant.

Activity and biodistribution of the exogenous HexD3 enzyme were further measured utilizing a hexosaminidase enzymatic activity assay. The principles of this assay involve the use of a naphthol compound (an artificial substrate of hexosaminidase HexA) to localize the presence of active recombinant hexosaminidase compounds, including HexD3. A subset of the 65-day (post-administration) samples were frozen without fixation, in preparation for this enzymatic assay.

15 μm, sagittal cut cryosections from the ipsilateral injection-side brain were fixed in a solution containing 2% formaldehyde, 0.2% glutaraldehyde and, and 0.01% Nonidet P40 substitute for 15 minutes at room temperature. Sections were incubated in the substrate naphthol AS-BI N-acetyl-β-glucosaminide (NAP). NAP was first dissolved in dimethylformamide, 50-100 μl). 6 mg/30-35 mL concentration of NAP was diluted in 1× citrate buffer (from a 20× concentration), pH 4.5 and incubated for 3 hours. Sections were then re-incubated in the NAP solution, pH 5.2, with 90 μL of hexazotized pararsaniline was used). Equal volumes of this pararosaniline solution (already in HCl) were mixed with equal volumes of a 4% (wt/vol) sodium nitrite solution). The slides were immersed in the pararosaniline reagent for 1-2 hours until a red color deposit formed.

To determine if naphthol signal localizes near neuronal cells, the same frozen sections were immunostained with antibodies against NeuN (MAB377, 1-100). For antigen retrieval, slides were immersed in Discovery CC1 solution for 30 minutes at 95° C. immediately after the enzymatic staining. The blocking buffer contained a 2% NDS, 0.1% BSA and 0.3% Triton solution in 1×TBS. Donkey anti-mouse IgG (H+L) highly cross-adsorbed secondary antibody conjugated to Alexa Fluor 488 (A-21202, 1-500) was used to detect anti-NeuN antibodies. Slides were mounted in DAPI Fluoromount to visualize cellular nuclei.

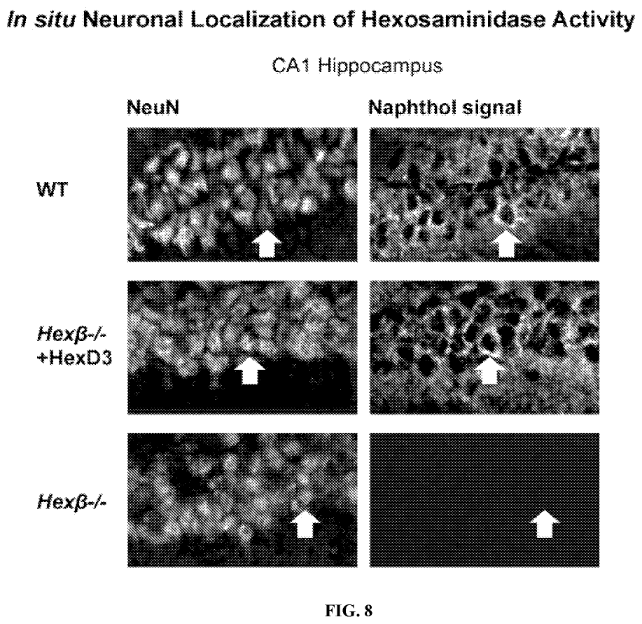

As neurons are a target cell type, the naphthol histologic stain was converted into a pseudo fluorescent signal to co-localize with immunofluorescent neuronal stain, NeuN. The naphthol stain can be seen throughout the tissue as well as in and around neurons in the hippocampus (FIG. 8). Using the described method, FIG. 8 shows HexD3 staining of the CA1 hippocampal region using NeuN and naphthol in the middle panel for Hexβ−/− with HexD3 ICV. The left column of photomicrographs shows NeuN staining of neuronal nuclei from CA1 region of the hippocampus. The right column of photomicrographs shows the same section co-stained with the metabolized naphthol signal. Arrows point to a single neuron in each section (FIG. 8).

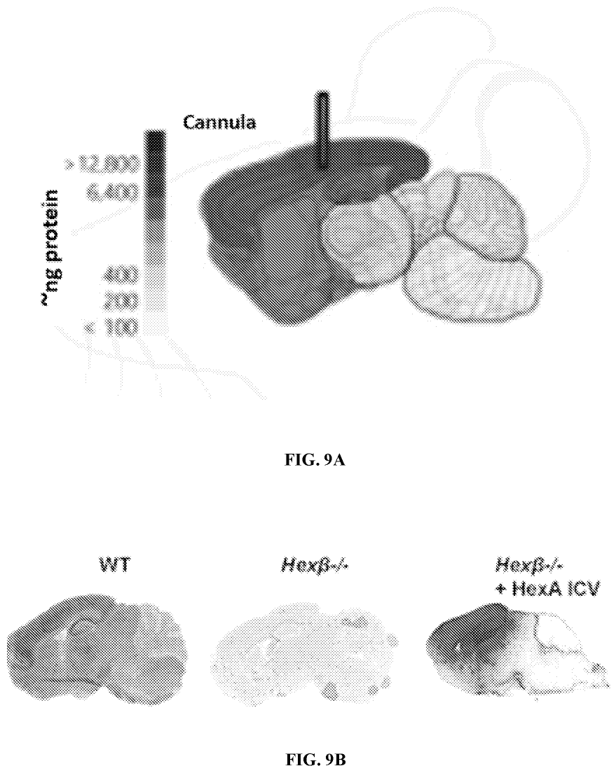

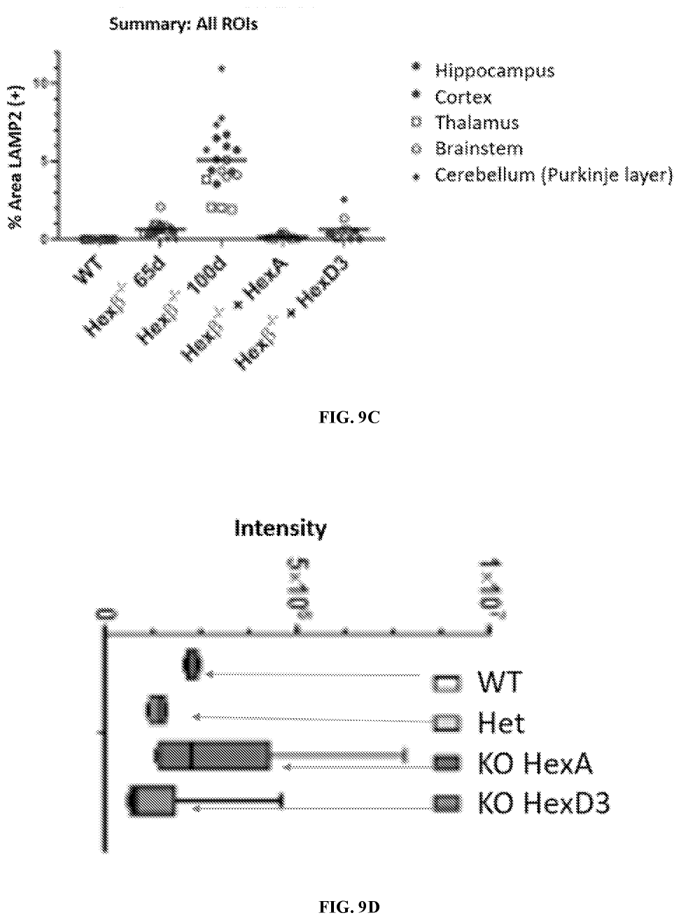

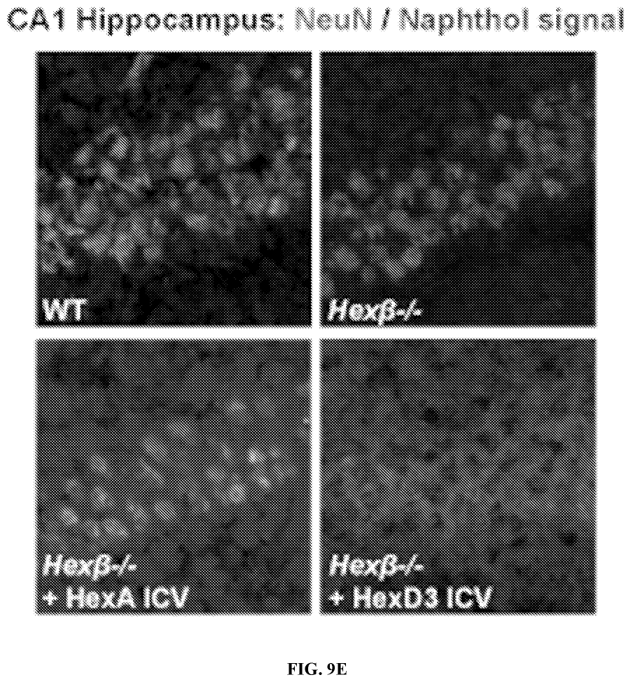

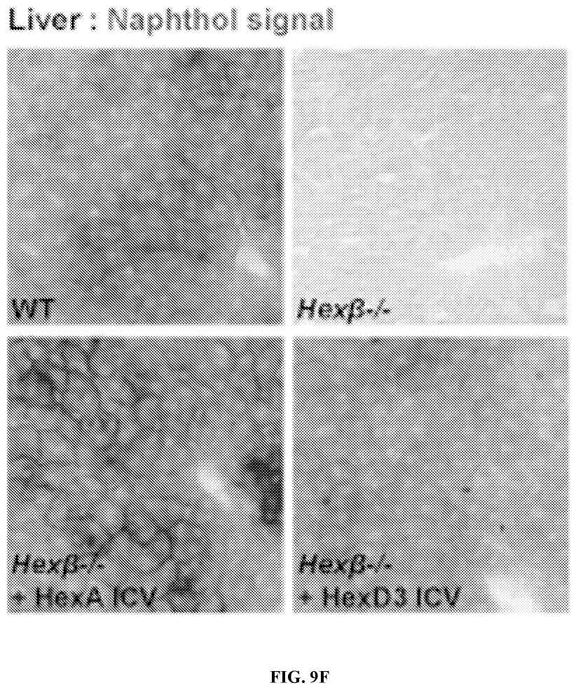

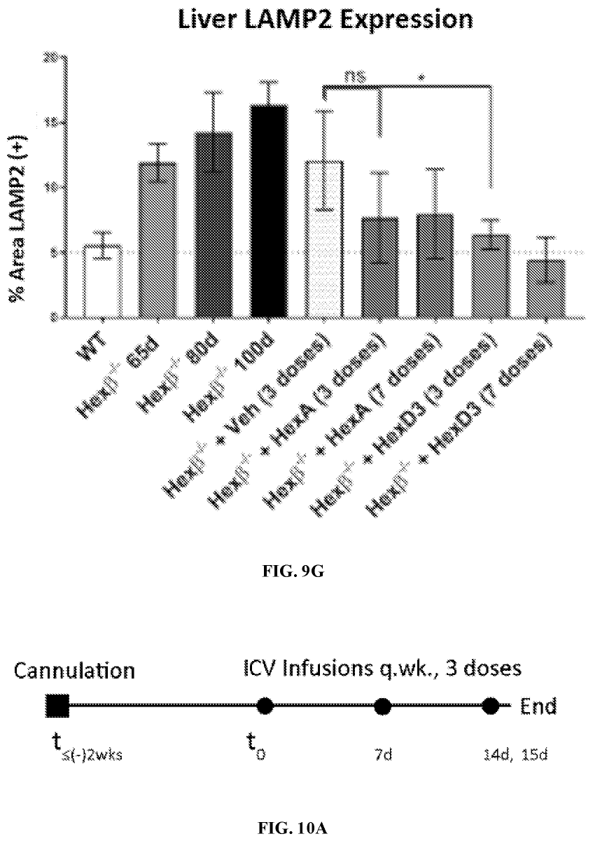

To understand the biodistribution of the active enzyme in the brain tissue following a bolus ICV injection, hexosaminidase enzyme was characterized by mass spectrometry from dissected brain regions. FIG. 9A shows the 24 hour-biodistribution of HexM after a single ICV injection. The heat map is based on mass spectrometry quantitation of total HexM (as a Hex enzyme example) ng/protein found in dissected brain regions. To assess the biodistribution following HexA ICV injection, brain tissue was processed as described after 3 q. wk of HexA. FIG. 9B shows the biodistribution after the HexA ICV regimen. Both naphthol and mass spectrometry showed that hind brain regions, including the cerebellum and brain stem, had the lowest exposure to the enzymes, seemingly below WT levels.

To understand the effect of LAMP2 levels in different regions of the brain, HexA or HexD3 ICV injections were performed and the hippocampus, cortex, thalamus, brainstem, and cerebellum LAMP2 levels were quantified. Despite the differing levels of enzyme (HexA and HexDA3) in various brain regions, LAMP2 was suppressed throughout the brain even in cerebellum and brain stem regions (FIG. 9C). Thus, the enzyme level in brain regions away from the site of administration were much lower but still high enough to restore lysosomal health. Even after repeated ICV injections (7 doses, once per week), HexA accumulated to a greater extent than HexD3 (FIG. 9D).

Neurons are the primary targets of Hex replacement therapy. To confirm co-localization of Hex activity with the immunofluorescent neuronal stain NeuN, the naphthol histologic stain was converted into a pseudo fluorescent signal (FIG. 9E). In the top left panel of FIG. 9E, the staining of CA1 wild-type hippocampus, where the naphthol stain could be seen throughout the tissue, in and around hippocampal neurons, is shown. Naphthol signal was reduced in a Hexβ−/− background as seen in the top right panel of FIG. 9E. A study using a neuronally expressed Hex transgene showed a similar pattern of Hex localization of activity in the hippocampus. Using the CA1 hippocampal layer, a strong signal with both HexA and HexD3 was seen, as shown in the bottom left and right panels of FIG. 9E.

In some instances, proteins in the CSF can diffuse into the blood stream and can be picked up by peripheral tissue such as the liver. To assess HexA and HexD3 distribution in the liver, liver samples were subjected to naphthol staining to determine the presence of HexA or HexD3 activity. By naphthol staining, HexA showed greater accumulation in the liver than HexD3 when comparing the bottom left (HexA ICV) and the bottom right (HexD3 ICV) panels of FIG. 9F. HexD3 distribution and level resembled WT most closely (FIG. 9F). The excess HexA staining concentrated around hepatocytes, possibly in the extracellular space. The gradual increase in liver lysosomal pathology, as ascertained by LAMP2 staining, was also prevented following ICV dosing with either HexA or HexD3 (FIG. 9G), though HexD3 enzyme appeared most effective reducing the expression of LAMP2 in the liver.

### Example 9: In Vivo Analysis of Ganglioside and Ganglioside Precursor Levels

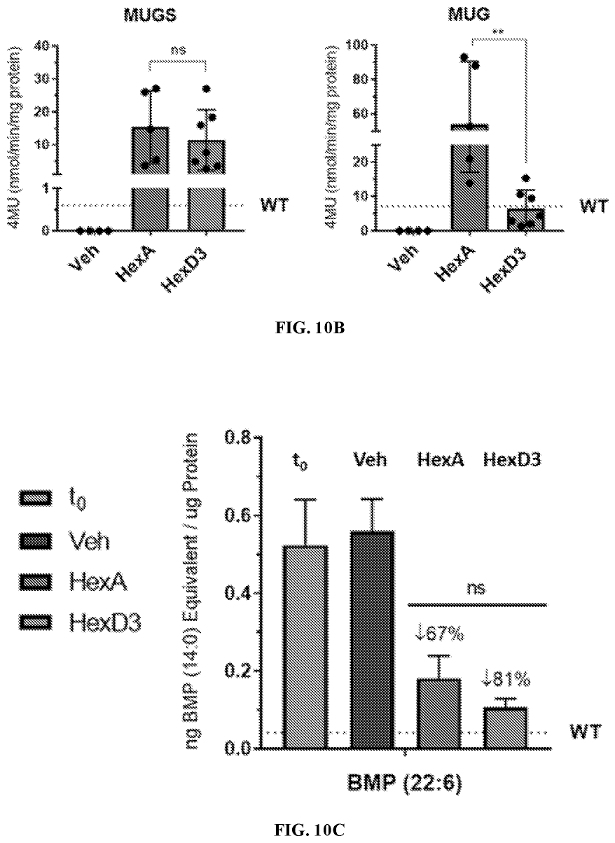

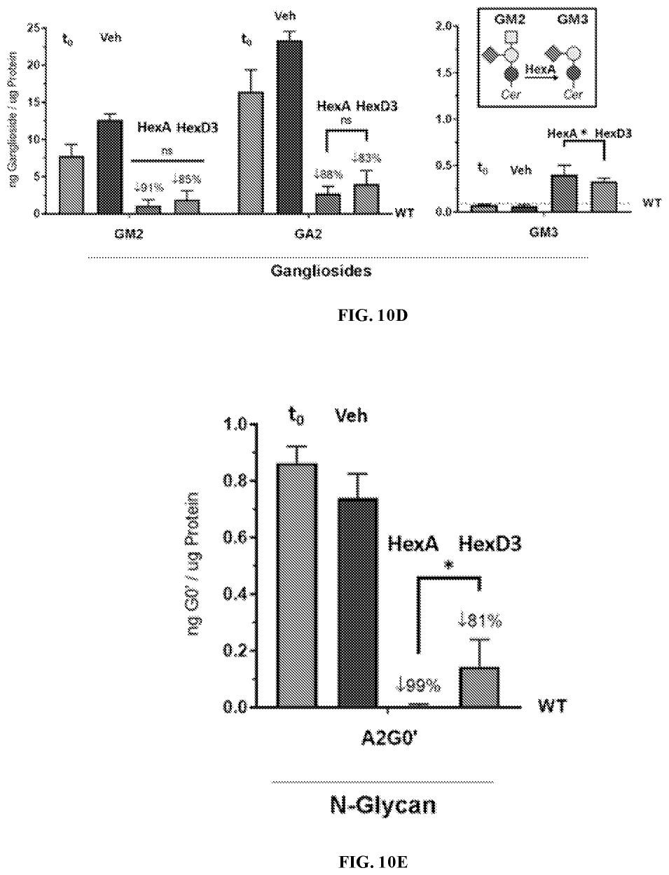

To investigate the in situ activity of HexD3 compared to HexA, both purified enzymes were delivered to the CNS by ICV injection into the Hexβ−/− KO mouse model of SD. Mice were injected at 2 months of age, at an asymptomatic stage and prior to CNS deterioration. Three doses, each one week apart, were delivered by ICV cannula (FIG. 10A). Twenty-four hours after the 3rd dose, Hex activity, the lysosomal biomarker lipid BMP, and Hex natural substrates were analyzed from brain homogenates. HexA showed elevated MUG and MUGS activity above WT levels (FIG. 10B). In comparison, MUG, but not MUGS, activity was significantly lower for HexD3 (FIG. 10B). The activity of HexD3 still reached WT levels on average. HexD3 showed significantly better reduction in lipid BMP (FIG. 10C) compared to treatment with vehicle alone. Furthermore, HexD3 was able to lower the gangliosides GM2 and GA2 as effectively as HexA (FIG. 10D), albeit to a lesser degree. Inversely, HexA performed better in raising GM3 levels, a product of GM2 metabolism according to the middle-right graph of FIG. 10D. HexA also significantly lowered levels of N-Glycan metabolites known to accumulate in SD, such as A2G0′, greater than HexD3 (FIG. 10D). While these parameters suggest HexD3 has overall weaker potency when it comes to substrate metabolism, the phospholipid BMP levels and the LAMP2 corrections mentioned above suggest HexD3's greater performance in improving lysosomal health.

### Sample Preparation

Brain tissue was homogenized in 500-1000 μL of water, with 1.4 mm ceramic beads using an Omni Bead Ruptor 24 Homogenizer. Protein concentration of the homogenate was determined using a Pierce BCA Assay kit. All homogenates were diluted with water to 4 μg protein/μL homogenate. An amount of sample equal to 200 μg of protein (50 μL diluted homogenate) was extracted with 500 μL 95/5 methanol/glacial acetic acid (v/v) for 2 hours, vortexing briefly every 20 min. Samples were spun down for 5 min. at 5,000 rpm and supernatant was transferred to a Nanosep Omega 10K centrifugal filter tube. Samples were centrifuged at 12,000 rpm until all sample had passed through the filter, approximately 30 min. Samples were injected directly to LC-MS/MS or stored at −20° C. until use.

### Standard Preparation

All samples were quantified using an external standard calibration curve. Standards were prepared in 95/5 methanol/glacial acetic acid (v/v). The ganglioside standard curve was a five-point standard curve (100-6.25 pg/μL) prepared as a mix of GM1, GM2, GM3, GA1, and GA2 standards. The BMP standard curve was a five-point standard curve (100-6.25 pg/μL) of BMP 14:0. All BMP 22:6 concentrations are reported as equivalents of BMP 14:0.

### LC-MS/MS Chromatography of Gangliosides

LC-MS/MS ganglioside analysis was performed on an Acquity UPLC system equipped with a Glycan BEH Amide column (1.7 μm, 2.1×150 mm) with Glycan BEH Amide VanGuard Pre-Column (1.7 μm, 2.1×5 mm) connected to a Xevo TQ-S micro Triple Quadrupole Mass Spectrometer. Mobile phase A was 5 mM ammonium acetate in 94.500 acetonitrile, 2.500 methanol, 2.5% water, and 0.50% formic acid. Mobile phase B was water. The LC elution gradient is detailed below in TABLE 12. The column was kept at 50° C. throughout the run. The samples were ionized by ESI in positive mode. The capillary voltage was set at 1.0 MV The desolvation temperature was set at 500° C. and the desolvation gas flow was 1000 L/hr. The cone voltage was set to 1 O and the cone gas flow was 10 L/hr. Two precursor-to-product ion transitions, detailed below, were monitored for each ganglioside, one of the 36:1 species and one for the 38:1 species (TABLE 13).

TABLE 12
LC Gradient Program

| | Total Time | Flow | Mobile | Mobile |
|---|---|---|---|---|
| Step | (min) | (mL/min) | Phase A (%) | Phase B (%) |
| 1 | 0.0 | 0.400 | 95 | 5 |
| 2 | 2.0 | 0.400 | 95 | 5 |
| 3 | 12.0 | 0.400 | 50 | 50 |
| 4 | 16.0 | 0.400 | 50 | 50 |
| 5 | 16.1 | 0.400 | 95 | 5 |
| 6 | 20.0 | 0.400 | 95 | 5 |

TABLE 13
Analyte Transitions

| | | | | | | Approx. |
|---|---|---|---|---|---|---|
| | | | | | Collision | Retention |
| | | Precursor | Product | Cone | Energy | Time |
| Analyte | Species | Ion | Ion | (V) | (V) | (min) |
| GM1 | GM1(36:1) | 1546.7 | 366.1 | 10 | 36 | 7.2 |
| | GM1(38:1) | 1574.7 | 366.1 | 10 | 36 | |
| GA1 | GA1(36:1) | 1255.7 | 366.1 | 10 | 24 | 6.6 |
| | GA1(38:1) | 1283.8 | 366.1 | 10 | 24 | |
| GM2 | GM2(36:1) | 1384.7 | 204.1 | 10 | 44 | 6.6 |
| | GM2(38:1) | 1412.7 | 204.1 | 10 | 44 | |
| GA2 | GA2(36:1) | 1093.6 | 264.3 | 10 | 54 | 5.3 |
| | GA2(38:1) | 1121.6 | 292.3 | 10 | 54 | |
| GM3 | GM3(36:1) | 1181.5 | 264.3 | 10 | 54 | 6.1 |
| | GM3(38:1) | 1209.6 | 292.3 | 10 | 54 | |

### LC-MS/MS Chromatography of BMP Phospholipid

LC-MS/MS BMIP phospholipid analysis was performed on an Acquity UPLC system equipped with a HlSS C18 column (1.8 μm, 1.0×150 mm) with 0.2 m in-line filter connected to a Xevo TQ-S micro Triple Quadrupole Mass Spectrometer using the conditions in TABLE 14 below and A2G0′ free glycan was measured by Sensi-Pro. Mobile phase A was 5 mM ammonium formate in 74% methanol, 25% water, and 10% formic acid. Mobile phase B was 5 mM ammonium formate in 99% methanol, and 1% formic acid. The LC elution gradient is detailed below. The column was kept at 50° C. throughout the run. The samples were ionized by ESI in negative mode. The capillary voltage was set at 3.5 kV. The desolvation temperature was set at 600° C. and the desolvation gas flow was 1000 L/hr. The cone voltage was set to 10V and the cone gas flow was 0 L/hr. One precursor to product ion transition was monitored for each BMP species, 14:0 and 22:6, as shown below (TABLE 15).

TABLE 14
LC Gradient Program

| | Total Time | Flow | Mobile | Mobile |
|---|---|---|---|---|
| Step | (min) | (mL/min) | Phase A (%) | Phase B (%) |
| 1 | 0.0 | 0.100 | 80 | 20 |
| 2 | 1.0 | 0.100 | 80 | 20 |
| 3 | 6.0 | 0.100 | 0 | 100 |
| 4 | 16.0 | 0.100 | 0 | 100 |
| 5 | 16.1 | 0.100 | 80 | 20 |
| 6 | 20.0 | 0.100 | 80 | 20 |

TABLE 15
Analyte Transitions

| | | | | Collision |
|---|---|---|---|---|
| | Precursor | Product | Cone | Energy |
| Analyte | Ion | Ion | (V) | (V) |
| BMP 14:0 | 665.4 | 227.2 | 10 | 32 |
| BMP 22:6 | 865.5 | 327.3 | 10 | 32 |

### Example 10: Survival Analysis of Sd Mice Treated with Variant Hexosaminidase Enzyme

To evaluate the ability of HexD3 and HexA to correct for the loss of hexosaminidase activity, purified HexD3 or HexA were administered to the brains of mice lacking hexosaminidase activity. A permanent ICV cannula into the left lateral ventricle was implanted in the mouse at 6 weeks of age.

After implantation, mice were single housed in wire free cages and provided floor access to food pellets and HydroGel cups (ClearH₂O). After 1 week of recovery and starting as early as 7-9 weeks of age, 100 μg bolus of enzyme in 5 μL volume was infused over a period of 5-10 minutes. An antihistamine injection of Benadryl at 5 mg/kg was given 10 minutes prior to ICV injections. ICV injections were continued weekly until the end of study or survival endpoint. For non-survival studies, animals were euthanized 24 hrs after the last ICV injection by cardiac puncture and blood collection followed by perfusion with PBS.

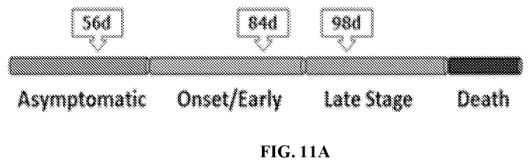

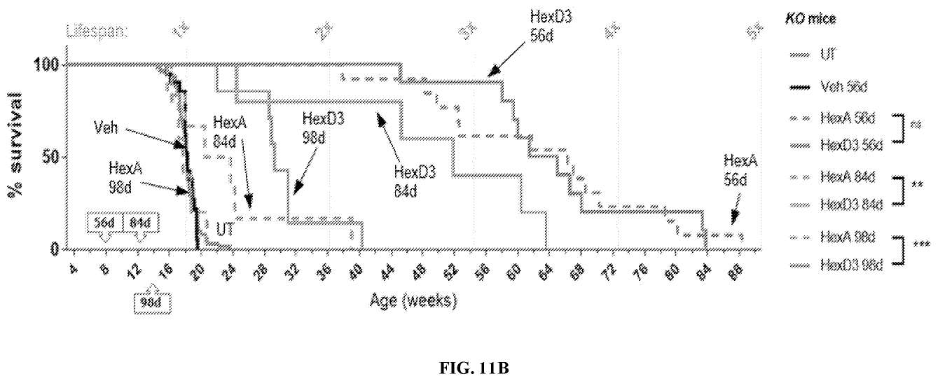

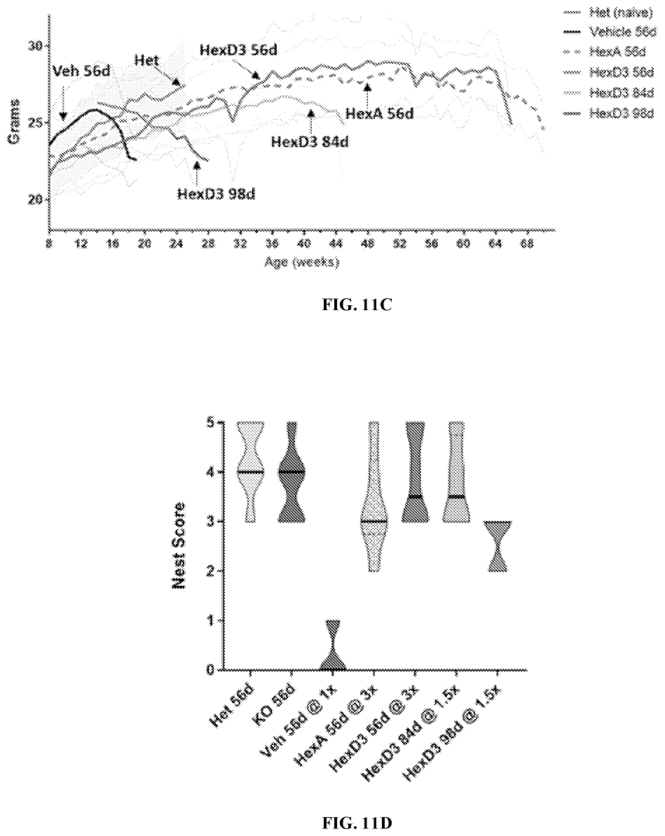

The health of the animals was monitored over time. As shown in FIG. 11B, administration of HexD3 or HexA to the brains of SD mice resulted in increased survival compared to mice treated with vehicle. The median survival age was extended by ˜3.5 fold the normal lifespan of the disease mice (untreated median, 127 days of age, n=60) when enzyme delivery, 100 μg weekly by ICV, was started during the asymptomatic period, at 56 days of age. Survival age of HexA treated animals (median, 464 days, n=13) compared to HexD3 treated ones (median, 442.5 days, n=10) was not significantly different (FIG. 11B). The behavior of these mice was grossly normal aside from some errors in hind limb placement. Two of the longest living SD animals [618 days (HexA treated) and 586 days (HexD3 treated)] lived nearly as long as the normal mouse lifespan (600-900 days of age) (FIG. 11C). The terminal phenotypes of these treated mice varied from sudden death to progressive decline in weight with increasing signs of spasticity though less severe effects on gross locomotor activity with age.

While the treatments of HexA and HexD3 displayed comparable effects when treatment was initiated at 56 days, surprising differences in treatments of HexA and HexD3 effects emerged when the starting age of treatment was delayed to either 84 or 98 days (FIG. 11A and FIG. 11B). These ages were chosen to mimic a treatment paradigm for early onset and late stage neurodegenerative pathology, i.e. elevated NF-L. When treatment was started at 84 days of age, the lifespan for HexD3 treated mice (median, 363 days, n=6) was significantly improved compared with HexA treated mice (median, 155 days, n=6). When treatment was started at 98 days of age, no effect was seen with HexA (median, 124 days, n=6) when compared to vehicle treatment (median, 128 days, n=20); whereas HexD3 treatment started at 98 days showed a significant survival benefit (median, 205 days, n=7), even greater than ICV HexA treatment started at 84 days of age (FIG. 11B).

Animals treated with HexD3 prior to 98 days of age maintained normal (or near normal) nest building activity, stable weight and motor activity for the duration of their prolonged lifespans (FIG. 11C-11E). Animals treated at 98 days of age had a different weight profile from those treated earlier than 98 days of age. In animals treated at 98 days, treatment with HexD3, slowed but did not ultimately change the continued trajectory of weight loss. In contrast, the progressive decline in nest building and motor activity did change more dramatically, evident from the time of treatment (FIG. 11E). However, these animals treated at 98 days with HexD3 could not recover to normal activity levels even after an extended period. At start of treatment, decline in motor activity halted or plateaued for the remainder of the animals' lifespan (FIG. 11F). Weight loss though slowed continued eventually meeting endpoint criteria (FIG. 11C).

### Example 11: Blood Neurofilament Light Chain as a Biomarker for Gm2 Neurodegeneration

Neurofilament light chain (NF-L) has been explored as a fluid marker of axonal neuron injury and is readily elevated in neurodegenerative disorders. This should also be true for classic lysosomal storage disorders with neurodegeneration. Plasma samples from KO mice at various ages were collected and assayed by Quanterix, which measures plasma NF-L. A minimum of 20 μL plasma was used from terminal bleed or tail vein nick and a minimum of 3 animals per group were analyzed.

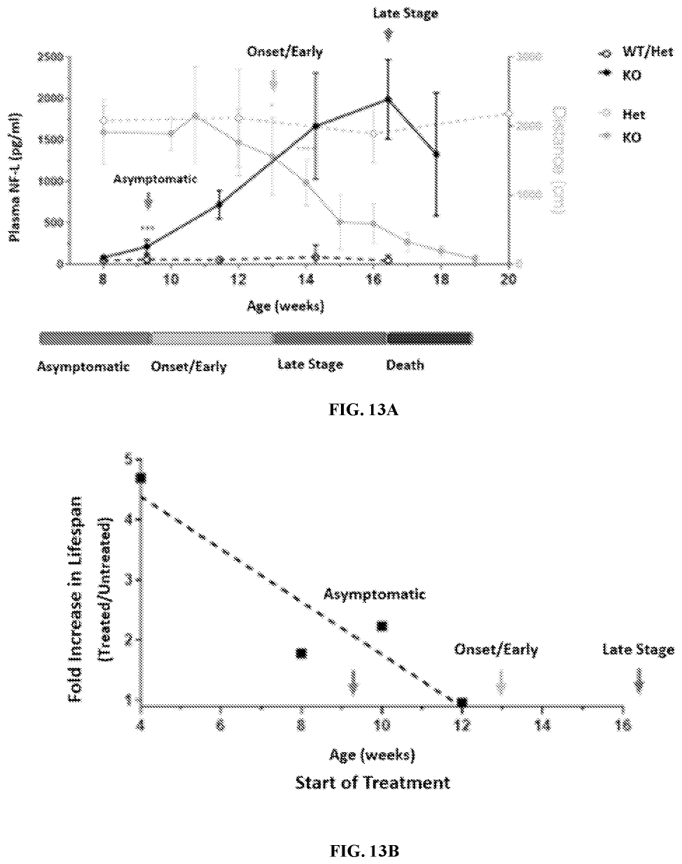

FIG. 13A shows a largely asymptomatic stage preceding rise in NF-L levels, followed by early onset neurodegeneration prior to significant signs of motor deficit, followed by late-stage pathology where NF-L levels reach their maximum and motor decline continues, and finally morbidity/death when euthanasia criteria is reached. An increase in NF-L plasma levels above WT was noted after 65 days of age (FIG. 13A). At around 100 days of age, NF-L levels in the SD mouse model rose to approximately 100× normal levels. These results demonstrated that NF-L is a sensitive and reliable marker of disease progression in the SD mouse model and may have clinical relevance to track neuronal injury in patients.

### Example 12: Suppression of Blood Nf-L Levels by Hex Icv Infusions

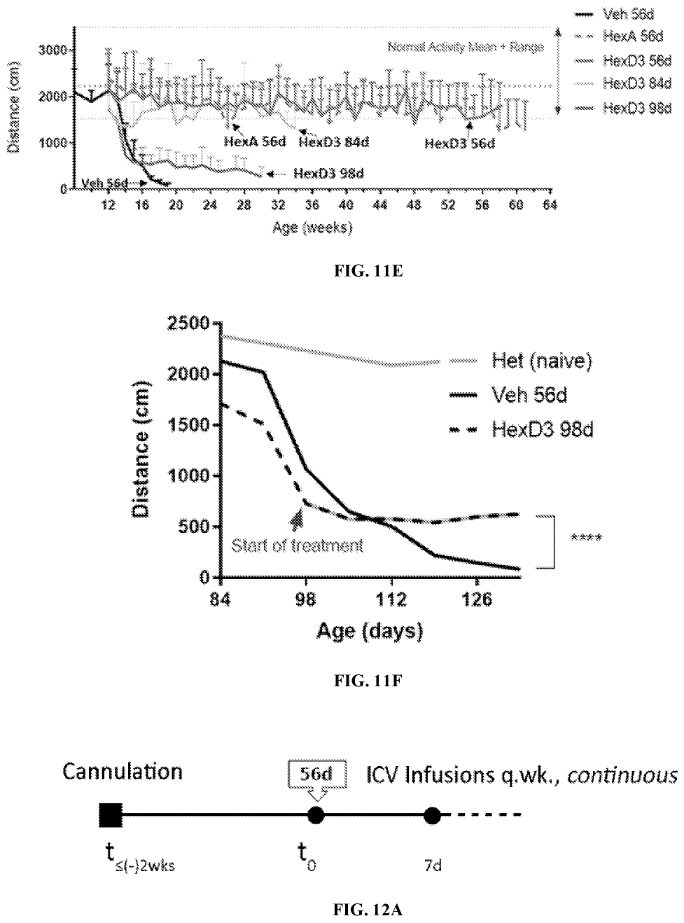

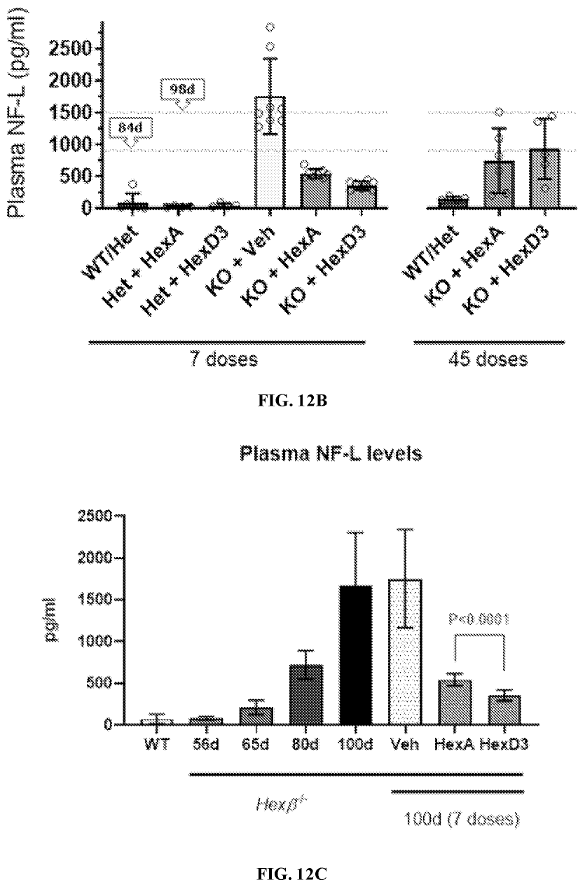

To ascertain treatment efficacy, blood NF-L levels were analyzed longitudinally. Diseased mice began treatment at 56 days of age, prior to a statistically significant rise in NF-L levels (FIGS. 12A, 13A). ICV infusions of either HexA or HexD3 were delivered weekly until euthanasia criteria were met. NF-L levels were checked after 7 doses and 45 doses through blood collections.

Both Hex enzymes partially suppressed NF-L levels after either 7 doses or when the mice were 98 days old (FIG. 12B). The levels remained below what would be expected for untreated 84-day-old KO mice in an early onset stage of the disease. After 45 doses in one-year-old mice, the NF-L levels did increase, but on average remained at a level expected for 84-day-old KO animals. The highest NF-L levels reported in treated one-year-old KO mice were at or below what would be expected for 98-day-old untreated KO mice in late stages of the disease (FIG. 12B). At 98 days of age, NF-L levels begin to peak in untreated SD mice (FIG. 13A). Most treated SD mice at one year of age exhibited signs of good health, enough to maintain stable weight (FIG. 11C) and normal nesting behavior (FIG. 11D). Other animals declined towards a survival endpoint and were euthanized. A threshold where continuous NF-L levels on or above the levels normally seen at 98 days of age may be most detrimental.

### Example 13: Disease Progression in a Mouse Model of Sd

NF-L has been explored as a marker of axonal neuron injury and is readily elevated in neurodegenerative disorders. In Parkinson's, Alzheimer's disease, and other neurodegenerative disorders, NF-L levels increase with disease onset and severity. NF-L blood levels were measured to monitor the neurodegenerative disease time course in the SD mouse model (FIG. 13A-13B). Plasma samples from SD (Hexβ KO) mice at various ages were collected and NF-L levels compared to WT or Het controls. No significant difference from normal was seen within the first 8 weeks of age (FIG. 13A). A small but significant elevation of NF-L was detected by week 9 (time point at 65 days of age). NF-L levels then continued to increase steadily and peak by week 16, achieving approximately 100× normal levels at 115 days of age. The average NF-L level dropped slightly in older mice approaching endpoint criteria (FIG. 13A).

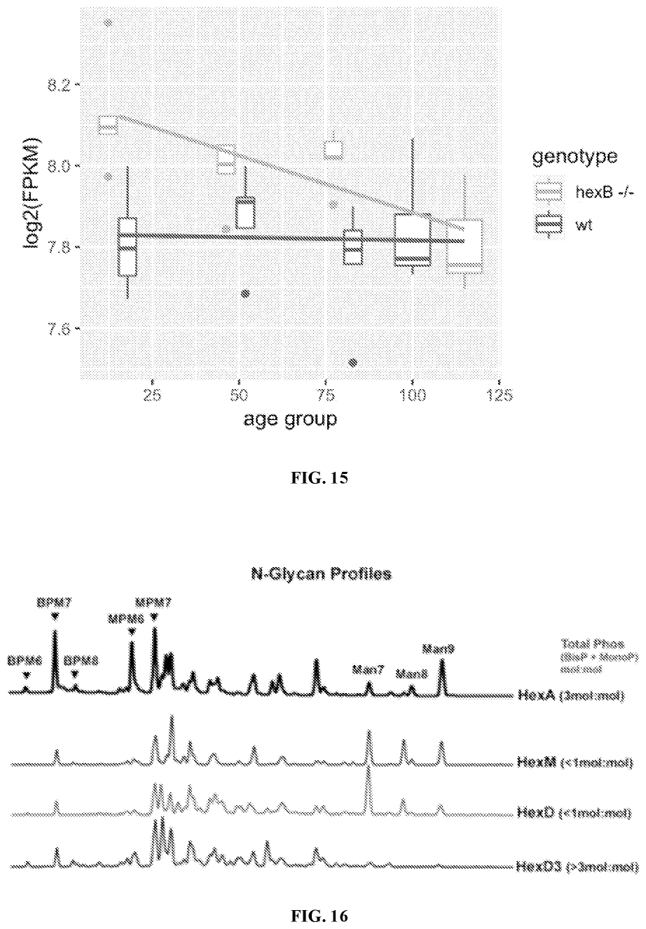

Combining both behavior phenotype and NF-L levels defined the stages of disease progression in SD mice. Locomotor activity, reported as distance, was measured in an open-field assay to track onset of neurological phenotype. SD mice younger than 65 days of age (FIG. 13A) had normal NF-L levels and locomotor activity. Thus, any age prior to NF-L rise was asymptomatic. A comparison with motor deficit, measured using open field activity monitoring, showed that the initial rise in NF-L at 9 weeks was evident prior to detection of significant differences in locomotor activity by 13 weeks of age (FIG. 13A, grey line: locomotor activity; black line: plasma NF-L). At the onset of NF-L level increase, but absent significant decline in motor activity, this range is considered to be disease onset or early stage (FIGS. 13A-13B). After 91 days of age, a significant decline in locomotor activity and a peak elevation of NF-L is seen at approximately 115 days of age (FIGS. 13A-13B). This age range is labeled as late stage disease. After the 115-day time point, SD mice approached endpoint criteria for euthanasia. NF-L levels appear to plateau or dip, possibly due to neuron loss at end of life and consequent decrease in a NF-L source (FIG. 13A). Whole brain tissues were assessed by RNA-seq. Box plots with regression line show FPKM, which is the normalized RNASeq expression value for NF-L. Brain tissue was sampled at 15, 50, 80 and 115 days of age. Note that at 115 days (week 16) blood protein levels peak while, RNA levels of NF-L drop (FIG. 15).

In order to capture the limited window of opportunity to rescue aged SD mice, median increase in lifespan as a fold change was compared between treated and untreated animals, against the age of the mice at time of viral injection. A rough negative linear correlation could be drawn (FIG. 13C). When overlaid with disease staging, it became evident that HexA gene therapy treatment had limited effect on disease progression after the onset of neurodegeneration (FIG. 13C) and failed to have any impact when the mice were nearing late stage disease (FIG. 13C).

TABLE 16 shown below is a summary comparing the activities of various β-hexosaminidase enzymes.

TABLE 16
β-Hexosaminidase enzyme summaries

| Hex isozyme (name) | HexA | HexM | HexD3 |
|---|---|---|---|
| Gene | HEXA/HEXB | Modified HEXA | Modified HEXA |
| Amino acid | None | S184K, P209Q, | S184K, P209Q, |
| substitutions in alpha | | NPVT 228-231 S-LS, | NPVT 228-231 S-LS, |
| subunit | | P429Q, KD 432-433 | P429Q, KD 432-433 |
| | | RK, I436K, N466A, | RK, I436K, N466A, |
| | | S491R, LTF 493-495 | S491R, LTF 493-495 |
| | | MDD, E498D, | MDD, E498D, |
| | | L508V, Q513A, NV | L508V, Q513A, NV |
| | | 518-519 YA, F521Y, | 518-519 YA, F521Y, |
| | | E523N | E523N, alpha 1-165 |
| | | | to beta 1-198 |
| Subunit composition | αβ | α₁′α₁′ | α₃′α₃′ |
| (dimer) | | | |
| M6P-type N-glycan/ | 3/7 | 2/6 | 4/8 |
| N-glycan | | | |
| M6P context (mol/mol | 2.5-3 | <1 | 3-3.5 |
| Hex) | | | |
| K uptake (nM) MUG | 3.3 ± 0.3 | 130 ± 5.4 | 5.8 ± 1.0 |
| activity | | | |
| Intracellular half-life | 14 d | 12 d | 14 d |
| (t_{1/2}) MUG activity | | | |
| Kₘ GM2AP [μM] | 14.5 ± 1.6 | 19.6 ± 2.5 | 13.7 ± 2.4 |
| Specific activity GM2 | 45.8 | 20.6 | 13.6 |
| [nmol GM3/min/mg] | | | |
| Specific activity MUG | 66.7 ± 7 | 31 ± 3 | 27 ± 2 |
| (μmol/min/mg) | | | |
| Specific activity | 23 ± 3 | 47 ± 4 | 39 ± 3 |
| MUGS (μmol/min/mg) | | | |
| Tm (° C.) | 57 ± 0.1 | 63 ± 0.1 | 61 ± 0.2 |
| Thermostability at | 1.5 d | 6 d | NA |
| 37° C. in vitro CSF (t_{1/2}) | | | |

### Example 14: Therapeutic Window to Late-Stage Treatment

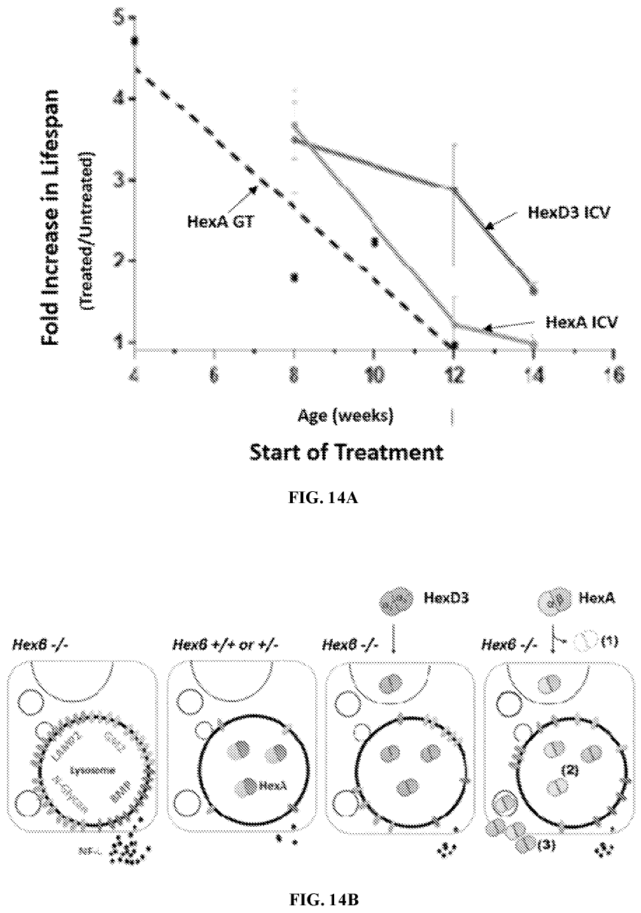

To compare treatment modalities, lifespan improvement versus the starting age of treatment in mice receiving ICV enzyme replacement therapy was plotted (FIG. 14A). HexA delivered by ICV showed a similar trendline to the extrapolated HexA gene therapy data, indicating that gene therapy or enzyme replacement by ICV exhibits little difference for HexA treatment. HexD3's trendline, on the other hand, showed a shift towards improved survival of SD mice when treatment was started at later ages indicating that the therapeutic window of treatment expands to late stages of the disease, but also indicates that the rescue potential is drug dependent. HexA and HexD3 performed equally well as therapeutics in a disease prevention paradigm but only HexD3 was able to modify disease when used in a treatment paradigm.

FIG. 14B is a model suggesting that that HexD3 may be more effective as a therapy for the following reasons: (1) Stability: HexA is potentially less stable in fluid compared to HexD3, (2) Homodimerization: HexA has added potential to recombine into isoenzymes HexS (α/α subunit) and HexB (β/β subunit), and (3) Accumulation: HexA accumulates in tissues to a greater extent than HexD3. Thus, despite the native HexA's effectiveness at normalizing GM2 and N-Glycans in vivo, HexD3 was better at normalizing lysosomal health, as judged by LAMP2 and phospholipid BMP levels. In both treatment cases, blood NF-L increase was prevented or reduced in the SD mouse model, demonstrating that NF-L can be used as a fluid biomarker for tracking neurological disease and rescue in GM2 disease.

Additional experiments were performed to assess the home cage behavior of mice after treatment with either HexA or HexD3. Mice treated with either HexA or HexD3 from 56 days of age showed grossly normal home cage behavior and activity. HexA and HexD3 treatment animals could not be easily distinguished from each other.

Mice treated with HexD3 from 56 days of age were also assessed for grooming and rearing activity. Mice placed in a confined space with a ledge (such as a shallow Nalgene cup) would attempt to rear to investigate their surroundings. This set-up allowed for visual inspection of behavior, foot pad position while rearing, grooming and ability to move around in a confined space. Treated mice showed normal grooming and rearing activity but with abnormal foot positioning, likely due to some neuronal degeneration. Worsening of the phenotype with age was not observed.

What is claimed is:

1. A recombinant β-hexosaminidase variant α subunit that forms a β-hexosaminidase variant α subunit homodimer comprising one or more amino acid sequence substitutions or deletions at positions corresponding to S184, P209, N228, P229, V230, T231, P429, K432, D433, 1436, N466, 5491, L493, T494, F495, E498, L508, Q513, N518, V519, F521, and E523 of native β-hexosaminidase α subunit of SEQ ID NO:1 and further comprising one or more amino acid sequence elements that increase cellular uptake of the β-hexosaminidase variant α subunit homodimer relative to a homodimer of SEQ ID NO: 6.

2. The recombinant β-hexosaminidase variant α subunit of claim 1, wherein the one or more amino acid sequence elements that increase cellular uptake are selected from the group consisting of amino acid sequence substitutions, additions, or deletions relative to the native β-hexosaminidase α subunit of SEQ ID NO:1.

3. The recombinant β-hexosaminidase variant α subunit of claim 1, wherein the β-hexosaminidase variant α subunit comprises one or more amino acid substitutions or deletions corresponding to S184K, P209Q, N228S, P229, V230L, T231S, P429Q, K432R, D433K, 1436K, N466A, S491R, L493M, T494D, F495D, E498D, L508V, Q513V, N518Y, V519A, F521Y, and E523N relative to the native β-hexosaminidase α subunit of SEQ ID NO:1.

4. The recombinant β-hexosaminidase variant α subunit of claim 3, wherein the β-hexosaminidase variant α subunit comprises at least five amino acid substitutions or deletions corresponding to S184K, P209Q, N228S, P229Δ V230L, T231S, P429Q, K432R, D433K, 1436K, N466A, S491R, L493M, T494D, F495D, E498D, L508V, Q513V, N518Y, V519A, F521Y, and E523N relative to the native β-hexosaminidase α subunit of SEQ ID NO:1.

5. The recombinant β-hexosaminidase variant α subunit of claim 4, wherein the β-hexosaminidase variant α subunit comprises at least ten amino acid substitutions or deletions corresponding to S184K, P209Q, N228S, P229Δ, V230L, T231S, P429Q, K432R, D433K, 1436K, N466A, S491R, L493M, T494D, F495D, E498D, L508V, Q513V, N518Y, V519A, F521Y, and E523N relative to the native β-hexosaminidase α subunit of SEQ ID NO:1.

6. The recombinant β-hexosaminidase variant α subunit of claim 5, wherein the β-hexosaminidase variant α subunit comprises at least fifteen amino acid substitutions or deletions corresponding to S184K, P209Q, N228S, P229Δ V230L, T231S, P429Q, K432R, D433K, 1436K, N466A, S491R, L493M, T494D, F495D, E498D, L508V, Q513V, N518Y, V519A, F521Y, and E523N relative to the native β-hexosaminidase α subunit of SEQ ID NO:1.

7. The recombinant β-hexosaminidase variant α subunit of claim 6, wherein the β-hexosaminidase variant α subunit comprises at least twenty amino acid substitutions or deletions corresponding to S184K, P209Q, N228S, P229, V230L, T231S, P429Q, K432R, D433K, 1436K, N466A, S491R, L493M, T494D, F495D, E498D, L508V, Q513V, N518Y, V519A, F521Y, and E523N relative to the native β-hexosaminidase α subunit of SEQ ID NO:1.

8. The recombinant β-hexosaminidase variant α subunit of claim 7, wherein the β-hexosaminidase variant α subunit comprises amino acid substitutions or deletions corresponding to S184K, P209Q, N228S, P229Δ, V230L, T231S, P429Q, K432R, D433K, I436K, N466A, S491R, L493M, T494D, F495D, E498D, L508V, Q513V, N518Y, V519A, F521Y, and E523N relative to the native β-hexosaminidase α subunit of SEQ ID NO: 1.

9. The recombinant β-hexosaminidase variant α subunit of claim 1, wherein the β-hexosaminidase variant α subunit comprises a first amino acid sequence comprising at least 85% sequence identity to SEQ ID NO: 11 and the one or more amino acid sequence elements that increase cellular uptake of the β-hexosaminidase variant α subunit relative to a homodimer of SEQ ID NO: 6 comprises a second amino acid sequence.

10. The recombinant β-hexosaminidase variant α subunit of claim 9, wherein the first amino acid sequence comprises at least 90% sequence identity to SEQ ID NO: 11.

11. The recombinant β-hexosaminidase variant α subunit of claim 10, wherein the first amino acid sequence comprises at least 95% sequence identity to SEQ ID NO: 11.

12. The recombinant β-hexosaminidase variant α subunit of claim 11, wherein the first amino acid sequence comprises at least 99% sequence identity to SEQ ID NO: 11.

13. The recombinant β-hexosaminidase variant α subunit of claim 12, wherein the first amino acid sequence comprises the amino acid sequence of SEQ ID NO: 11.

14. The recombinant β-hexosaminidase variant α subunit of claim 9, wherein the second amino acid sequence is N-terminal to the first amino acid sequence.

15. The recombinant β-hexosaminidase variant α subunit of claim 9, wherein the second amino acid sequence comprises at least 20 contiguous amino acid residues of SEQ ID NO: 2.

16. The recombinant β-hexosaminidase variant α subunit of claim 9, wherein the second amino acid sequence comprises at least 100 contiguous amino acid residues of SEQ ID NO: 12.

17. The recombinant β-hexosaminidase variant α subunit of claim 9, wherein the second amino acid sequence comprises at least 100 contiguous amino acid residues of SEQ ID NO: 13.

18. The recombinant β-hexosaminidase variant α subunit of claim 9, wherein the second amino acid sequence comprises at least 150 contiguous amino acid residues of SEQ ID NO: 13.

19. The recombinant β-hexosaminidase variant α subunit of claim 9, wherein the second amino acid sequence comprises at least 85% sequence identity to SEQ ID NO: 13.

20. The recombinant β-hexosaminidase variant α subunit of claim 9, wherein the second amino acid sequence comprises at least 90% sequence identity to SEQ ID NO: 13.

21. The recombinant β-hexosaminidase variant α subunit of claim 9, wherein the second amino acid sequence comprises at least 95% sequence identity to SEQ ID NO: 13.

22. The recombinant β-hexosaminidase variant α subunit of claim 9, wherein the second amino acid sequence comprises the amino acid sequence of SEQ ID NO: 13.

23. The recombinant β-hexosaminidase variant α subunit of claim 1, wherein the β-hexosaminidase variant α subunit comprises an amino acid sequence with at least 90% sequence identity to SEQ ID NO: 3.

24. The recombinant β-hexosaminidase variant α subunit of claim 23, wherein the β-hexosaminidase variant α subunit comprises an amino acid sequence with at least 95% sequence identity to SEQ ID NO: 3.

25. The recombinant β-hexosaminidase variant α subunit of claim 24, wherein the β-hexosaminidase variant α subunit comprises an amino acid sequence with at least 99% sequence identity to SEQ ID NO: 3.

26. The recombinant β-hexosaminidase variant α subunit of claim 25, wherein the β-hexosaminidase variant α subunit comprises the amino acid sequence according to SEQ ID NO: 3.

27. The recombinant β-hexosaminidase variant α subunit of claim 23, wherein the β-hexosaminidase variant α subunit comprises at least 500 contiguous amino acids of an amino acid sequence according to SEQ ID NO: 3.

28. The recombinant β-hexosaminidase variant α subunit of claim 27, wherein the β-hexosaminidase variant α subunit comprises at least 525 contiguous amino acids of an amino acid sequence according to SEQ ID NO: 3.

29. The recombinant β-hexosaminidase variant α subunit of claim 28, wherein the β-hexosaminidase variant α subunit comprises at least 550 contiguous amino acids of an amino acid sequence according to SEQ ID NO: 3.

30. The recombinant β-hexosaminidase variant α subunit of claim 23, wherein the β-hexosaminidase variant α subunit comprises at least 500 contiguous amino acids of an amino acid sequence according to SEQ ID NO: 3, and at least 95% sequence identity to the at least 500 contiguous amino acids.

31. The recombinant β-hexosaminidase variant α subunit of claim 23, wherein the β-hexosaminidase variant α subunit comprises at least 525 contiguous amino acids of an amino acid sequence according to SEQ ID NO: 3, and at least 95% sequence identity to the at least 525 contiguous amino acids.

32. The recombinant β-hexosaminidase variant α subunit of claim 23, wherein the β-hexosaminidase variant α subunit comprises at least 550 contiguous amino acids of an amino acid sequence according to SEQ ID NO: 3, and at least 95% sequence identity to the at least 550 contiguous amino acids.

33. The recombinant β-hexosaminidase variant α subunit of claim 23, wherein the β-hexosaminidase variant α subunit comprises an amino acid sequence with at least 85% sequence identity to SEQ ID NO: 3.

34. The recombinant β-hexosaminidase variant α subunit of claim 1, wherein the β-hexosaminidase variant α subunit homodimer comprises an increased mannose-6-phosphorylation (M6P) relative to a homodimer of SEQ ID NO: 6.

35. The recombinant β-hexosaminidase variant α subunit of claim 34, wherein the β-hexosaminidase variant α subunit homodimer has at least 3 M6P sites occupied by M6P per homodimer.

36. The recombinant β-hexosaminidase variant α subunit of claim 34, wherein the β-hexosaminidase variant α subunit homodimer has at least 4 M6P sites occupied by M6P per homodimer.

37. The recombinant β-hexosaminidase variant α subunit of claim 1, wherein the β-hexosaminidase variant α subunit homodimer exhibits GM2 ganglioside hydrolysis activity in the presence of GM2-activator protein.

38. The recombinant β-hexosaminidase variant α subunit of claim 1, wherein the cellular uptake of the β-hexosaminidase variant α subunit homodimer is increased by at least 2-fold, 5-fold, 10-fold, 20-fold, or 25-fold relative to a homodimer of SEQ ID NO: 6.

39. The recombinant β-hexosaminidase variant α subunit of claim 38, wherein the cellular uptake of the variant the β-hexosaminidase variant α subunit homodimer is assessed by uptake of cation independent mannose-6-phosphate receptor (CI-MPR) in a competition assay of the homodimer and mannose-6-phosphate in human fibroblasts from SD patients.

40. The recombinant β-hexosaminidase variant α subunit of claim 1, wherein the β-hexosaminidase variant α subunit homodimer has increased thermal stability relative to a heterodimer of SEQ ID NO: 1 and SEQ ID NO: 2.

41. The recombinant β-hexosaminidase variant α subunit of claim 40, wherein the β-hexosaminidase variant α subunit homodimer has a melting point (Tm) of about 60° C. to about 63° C.

42. The recombinant β-hexosaminidase variant α subunit of claim 1, wherein the β-hexosaminidase variant α subunit homodimer increases lifespan of a Hexb knockout mouse when administered at least age 80 days of age as compared to a heterodimer of SEQ ID NO: 1 and SEQ ID NO: 2 that is administered to a Hexb knockout mouse under substantially equivalent assay conditions and age.

43. The recombinant β-hexosaminidase variant α subunit of claim 1, wherein the β-hexosaminidase variant α subunit homodimer increases lifespan by at least 2-fold of a Hexb knockout mouse when administered at least 80 days of age as compared to a heterodimer of SEQ ID NO: 1 and SEQ ID NO: 2 that is administered to a Hexb knockout mouse under substantially equivalent assay conditions and age.

44. A homodimer comprising the recombinant β-hexosaminidase variant α subunit according to claim 1.

45. A pharmaceutical composition comprising the homodimer according to claim 44, and one or more pharmaceutically acceptable excipients.

46. The pharmaceutical composition of claim 45, wherein the one or more pharmaceutically acceptable excipients comprise sodium phosphate, sodium chloride, potassium chloride, magnesium chloride, and calcium chloride.

47. The pharmaceutical composition of claim 46, comprising 1 mM sodium phosphate, 148 sodium chloride, 3 mM potassium chloride, 0.8 mM magnesium chloride, 1.4 mM calcium chloride, pH 7.2.

48. The pharmaceutical composition of claim 46, wherein the homodimer comprising the recombinant β-hexosaminidase variant α subunit is formulated at a concentration of about 20 mg/ml.

49. A method of ameliorating the symptoms or slowing progression of TSD in a subject having TSD comprising administering to the subject an effective amount of the pharmaceutical composition of claim 45.

50. A method of ameliorating the symptoms or slowing progression of SD in a subject having SD comprising administering to the subject an effective amount of the pharmaceutical composition of claim 45.

51. The method of claim 49 or 50, wherein the pharmaceutical composition is administered to the subject intracerebroventricularly.

52. The method of claim 49 or 50, wherein the pharmaceutical composition is administered to the subject intracerebroventricularly weekly.

53. The method of claim 49 or 50, wherein the pharmaceutical composition is administered every other week.

54. The method of claim 49 or 50, wherein the pharmaceutical composition is administered at regular intervals, wherein the regular intervals are greater than every other week.

55. A nucleic acid encoding the homodimer comprising the recombinant β-hexosaminidase variant α subunit of claim 24.

56. A vector comprising the nucleic acid of claim 55 and one or more gene regulatory regions.

57. The vector of claim 56, further comprising adeno-associated virus (AAV) inverted terminal repeats (ITR) at both the 5′ and 3′ end of the vector.

58. An AAV viral particle comprising the vector of claim 57.

59. A method of ameliorating the symptoms or slowing disease progression of TSD in a subject having TSD comprising administering an effective amount of the AAV viral particle of claim 58 to the subject having TSD.

60. A method of ameliorating the symptoms or slowing disease progression of SD in a subject having TSD comprising administering an effective amount of the AAV viral particle of claim 58 to the subject having SD.

61. A method of reducing GM2 ganglioside accumulation in a subject having TSD or SD comprising administering an effective amount of the pharmaceutical composition of claim 45 to a subject having TSD or SD.

62. A method of reducing the level of a biomarker of disease progression of TSD in a subject having TSD comprising administering an effective amount of the pharmaceutical composition of claim 45 to a subject having TSD.

63. A method of reducing the level of a biomarker of disease progression of TSD in a subject having TSD comprising administering an effective amount of the AAV viral particle of claim 58 to a subject having TSD.

64. A method of reducing the level of a biomarker of disease progression of SD in a subject having SD comprising administering an effective amount of the pharmaceutical composition of claim 45 to a subject having or SD.

65. A method of reducing the level of a biomarker of disease progression of SD in a subject having SD comprising administering an effective amount of the viral particle of claim 58 to a subject having or SD.

66. The method of claim 63 or 64, wherein the biomarker of disease progression of TSD or SD is a GM2 and GA2, A2G0′ containing beta-linked terminal N-acetyl-D-hexosamine, BMP(22:6) phospholipid (Bis[monoacylglycero]phosphate)) or neurofilament light chain or a free glycan.

67. The method of claim 49, wherein the subject is pre-symptomatic for TSD.

68. The method of claim 49, wherein the subject has a TSD-associated mutation.

69. The method of any one of claim 49, wherein the subject has an elevated level of a biomarker of TSD relative to a control level of the biomarker of TSD.

70. The method of claim 69, wherein the elevated level of a biomarker of TSD is a measurement of the biomarker of TSD obtained from a sample from the subject.

71. The method of claim 70, wherein the control level of the biomarker of TSD is a measurement of the biomarker of TSD from a sample from a subject that does not have a TSD-associated mutation.

72. The method of claim 71, wherein the sample from the subject and the sample from the subject that does not have a TSD-associated mutation are quantified using liquid chromatography and mass spectrometry.

73. The method of any one of claim 72, wherein the sample from the subject and the sample from the subject that does not have a TSD-associated mutation are obtained from the CSF or blood.

74. The method of any one of claim 72, wherein the sample from the subject and the sample from the subject that does not have a TSD-associated mutation are blood plasma samples.

75. The method of claim any one of claim 69, wherein the elevated level of the biomarker of the TSD is elevated by at least 100% to 4000% relative to the control level of the biomarker of TSD.

76. The method of any one of claim 69, wherein the elevated level of the biomarker of the TSD is elevated by at least 2× to 100× relative to the control level of the biomarker of TSD.

77. The method of any one of claim 69, wherein the biomarker is GM2, GA2, A2G0′ containing beta-linked terminal N-acetyl-D-hexosamine, BMP(22:6) phospholipid (Bis[monoacylglycero]phosphate)), neurofilament light chain or a free glycan.

78. The method of claim 50, wherein the subject is pre-symptomatic for SD.

79. The method of claim 50 or claim 78, wherein the subject has a SD-associated mutation.

80. The method of any one of claim 50, wherein the subject has an elevated level of a biomarker of SD relative to a control level of the biomarker of SD.

81. The method of claim 80, wherein the elevated level of a biomarker of SD is a measurement of the biomarker of SD obtained from a sample from the subject.

82. The method of claim 81, wherein the control level of the biomarker of SD is a measurement of the biomarker of SD from a sample from a subject that does not have a SD-associated mutation.

83. The method of claim 82, wherein the sample from the subject and the sample from the subject that does not have a SD-associated mutation are quantified using liquid chromatography and mass spectrometry.

84. The method of any one of claim 83, wherein the sample from the subject and the sample from the subject that does not have a SD-associated mutation are obtained from the CSF or blood.

85. The method of any one of claim 83, wherein the sample from the subject and the sample from the subject that does not have a SD-associated mutation are blood plasma samples.

86. The method of any one of claim 80, wherein the elevated level of the biomarker of the SD is elevated by at least 100% to 4000% relative to the control level of the biomarker of SD.

87. The method of any one of claim 80, wherein the elevated level of the biomarker of the SD is elevated by at least 2× to 100× relative to the control level of the biomarker of SD.

88. The method of any one of claim 80, wherein the biomarker is GM2, GA2, A2G0′ containing beta-linked terminal N-acetyl-D-hexosamine, BMP(22:6) phospholipid (Bis[monoacylglycero]phosphate)), neurofilament light chain or a free glycan.


SEQUENCE LISTING (19 sequences)

SEQ ID NO: 1 - protein, 529 aa, Unknown
  Description of Unknown HexA alpha subunit sequence
MTSSRLWFSLLLAAAFAGRATALWPWPQNFQTSDQRYVLYPNNFQFQYDVSSAAQPGCSVLDEAFQRYRDLLFGSGSWPRPYLTGKRHTLEKNVLVVSVVTPGCNQLPTLESVENYTLTINDDQCLLLSETVWGALRGLETFSQLVWKSAEGTFFINKTEIEDFPRFPHRGLLLDTSRHYLPLSSILDTLDVMAYNKLNVFHWHLVDDPSFPYESFTFPELMRKGSYNPVTHIYTAQDVKEVIEYARLRGIRVLAEFDTPGHTLSWGPGIPGLLTPCYSGSEPSGTFGPVNPSLNNTYEFMSTFFLEVSSVFPDFYLHLGGDEVDFTCWKSNPEIQDFMRKKGFGEDFKQLESFYIQTLLDIVSSYGKGYVVWQEVFDNKVKIQPDTIIQVWREDIPVNYMKELELVTKAGFRALLSAPWYLNRISYGPDWKDFYIVEPLAFEGTPEQKALVIGGEACMWGEYVDNTNLVPRLWPRAGAVAERLWSNKLTSDLTFAYERLSHFRCELLRRGVQAQPLNVGFCEQEFEQT

SEQ ID NO: 2 - protein, 555 aa, Unknown
  Description of Unknown HexA beta subunit sequence
MELCGLGLPRPPMLLALLLATLLAAMLALLTQVALVVQVAEAARAPSVSAKPGPALWPLPLSVKMTPNLLHLAPENFYISHSPNSTAGPSCTLLEEAFRYHGYIFGFYKWHHEPAEFQAKTQVQQLLVSITLQSECDAFPNISSDESYTLLVKEPVAVLKANRVWGALRGLETFSQLVYQDSYGTFTINESTIIDSPRFSHRGILIDTSRHYLPVKIILKTLDAMAFNKFNVLHWHIVDDQSFPYQSITFPELSNKGSYSLSHVYTPNDVRMVIEYARLRGIRVLPEFDTPGHTLSWGKGQKDLLTPCYSRQNKLDSFGPINPTLNTTYSFLTTFFKEISEVFPDQFIHLGGDEVEFKCWESNPKIQDFMRQKGFGTDFKKLESFYIQKVLDIIATINKGSIVWQEVFDDKAKLAPGTIVEVWKDSAYPEELSRVTASGFPVILSAPWYLDLISYGQDWRKYYKVEPLDFGGTQKQKQLFIGGEACLWGEYVDATNLTPRLWPRASAVGERLWSSKDVRDMDDAYDRLTRHRCRMVERGIAAQPLYAGYCNHENM

SEQ ID NO: 3 - protein, 561 aa, Artificial Sequence
  Description of Artificial Sequence Synthetic polypeptide
MELCGLGLPRPPMLLALLLATLLAAMLALLTQVALVVQVAEAARAPSVSAKPGPALWPLPLSVKMTPNLLHLAPENFYISHSPNSTAGPSCTLLEEAFRRYHGYIFGFYKWHHEPAEFQAKTQVQQLLVSITLQSECDAFPNISSDESYTLLVKEPVAVLKANRVWGALRGLETFSQLVYQDSYGTFTINESTIIDSPRFPHRGLLLDTSRHYLPLKSILDTLDVMAYNKLNVFHWHLVDDQSFPYESFTFPELMRKGSYSLSHIYTAQDVKEVIEYARLRGIRVLAEFDTPGHTLSWGPGIPGLLTPCYSGSEPSGTFGPVNPSLNNTYEFMSTFFLEVSSVFPDFYLHLGGDEVDFTCWKSNPEIQDFMRKKGFGEDFKQLESFYIQTLLDIVSSYGKGYVVWQEVFDNKVKIQPDTIIQVWREDIPVNYMKELELVTKAGFRALLSAPWYLNRISYGQDWRKFYKVEPLAFEGTPEQKALVIGGEACMWGEYVDATNLVPRLWPRAGAVAERLWSNKLTRDMDDAYDRLSHFRCELVRRGVAAQPLYAGYCNQEFEQT

SEQ ID NO: 4 - DNA, 1587 bp, Unknown
  Description of Unknown HexA alpha subunit sequence
atgacttcctcccgcctttggttctccctcctgcttgccgctgccttcgccggacgcgccaccgccctgtggccgtggcctcagaacttccagactagcgaccaaagatacgtgctgtacccgaacaacttccagtttcaatacgacgtcagcagcgccgcccagcccggctgctcggtgctcgatgaggcattccagcggtaccgggatctcttgttcggttccggatcatggcctcggccgtacctcactggaaagaggcacactctcgaaaagaacgtcctggtggtgtccgtggtcacccctggctgcaatcagctgcccaccctggaatcagtggagaactacactttgaccatcaacgatgaccaatgcctgctgctgtcggagactgtgtggggtgccctgcgcgggctggaaacctttagccaactggtctggaagtcagccgaggggaccttcttcattaacaagaccgaaatcgaggacttccctcggttcccgcaccgcggcttgctgctggatacctcgcggcactatctgccactgtcctccattctcgataccctggacgtgatggcctacaacaagctgaacgtgttccactggcatctcgtggacgacccatcctttccctacgagtccttcaccttccctgagttgatgagaaagggctcctacaaccccgtgacccatatctacacggctcaggacgtgaaggaagtgatcgaatatgcccggctgagagggattagggtgctggcagaatttgacaccccgggacacaccctgtcgtggggccctggtatcccgggcctgctgactccgtgctactccggctccgagccatccggaaccttcggacctgtgaatccctccctgaacaacacttacgagttcatgtccaccttcttcctggaagtgtcgagcgtgttccccgacttctacctccacctcggtggcgacgaagtcgatttcacttgctggaagtctaaccccgagatccaagatttcatgcgaaagaaaggattcggagaggactttaagcagctggagtccttctacatccaaaccctgctggacattgtgtcatcgtatggaaagggatacgtggtgtggcaggaagtgtttgacaataaggtcaaaattcagcccgatacaatcatccaagtctggcgcgaagatatccccgtgaactacatgaaagaactggaactggtcacgaaggctggattcagggcgcttctgagcgccccttggtacttgaaccggattagctacggcccggactggaaggacttctacatcgtcgaacctctggccttcgagggaacccccgagcagaaggccttggtgatcggcggcgaagcctgtatgtggggggaatacgtggacaacaccaacctggtgccgcgcctgtggccgagagcgggagcagtggccgagcggctctggtcgaacaagctgacttccgacctcaccttcgcatacgagagactgagccacttccgctgcgaacttcttcggcggggggtgcaggcccagccgctcaacgtcgggttctgtgaacaggagttcgagcagacc

SEQ ID NO: 5 - DNA, 1670 bp, Unknown
  Description of Unknown HexA beta subunit sequence
atggagctgtgcgggctggggctgccccggccgcccatgctgctggcgctgctgttggcgacactgctggcggcgatgttggcgctgctgactcaggtggcgctggtggtgcaggtggcggaggcggctcgggccccgagcgtctcggccaagccggggccggcgctgtggcccctgccgctcttggtgaagatgaccccgaacctgctgcatctcgccccggagaacttctacatcagccacagccccaattccacggcgggcccctcctgcaccctgctggaggaagcgtttcgacgatatcatggctatatttttggtttctacaagtggcatcatgaacctgctgaattccaggctaaaacccaggttcagcaacttcttgtctcaatcacccttcagtcagagtgtgatgctttccccaacatatcttcagatgagtcttatactttacttgtgaaagaaccagtggctgtccttaaggccaacagagtttggggagcattacgaggtttagagacctttagccagttagtttatcaagattcttatggaactttcaccatcaatgaatccaccattattgattctccaaggttttctcacagaggaattttgattgatacatccagacattatctgccagttaagattattcttaaaactctggatgccatggcttttaataagtttaatgttcttcactggcacatagttgatgaccagtctttcccatatcagagcatcacttttcctgagttaagcaataaaggaagctattctttgtctcatgtttatacaccaaatgatgtccgtatggtgattgaatatgccagattacgaggaattcgagtcctgccagaatttgatacccctgggcatacactatcttggggaaaaggtcagaaagacctcctgactccatgttacagtagacaaaacaagttggactcttttggacctataaaccctactctgaatacaacatacgcttccttactacatttttcaaagaaattagtgaggtgtttccagatcaattcattcatttgggaggagatgaagtggaatttaaatgttgggaatcaaatccaaaaattcaagatttcatgaggcaaaaaggctttggcacagattttaagaaactagaatctttctacattcaaaaggttttggatattattgcaaccataaacaagggatccattgtctggcaggaggtttttgatgataaagcaaagcttgcgccgggcacaatagttgaagtatggaaagacagcgcatatcctgaggaactcagtagagtcacagcatctggcttccctgtaatcctttctgctccttggtacttagatttgattagctatggacaagattggaggaaatactataaagtggaacctcttgattttggcggtactcagaaacagaaacaacttttcattggtggagaagcttgtctatggggagaatatgtggatgcaactaacctcactccaagattatggcctcgggcaagtgctgttggtgagagactctggagttccaaagatgtcagagatatggatgacgcctatgacagactgacaaggcaccgctgcaggatggtcgaacgtggaatagctgcacaacctctttatgctggatattgtaaccatgagaacatgtaa

SEQ ID NO: 6 - protein, 528 aa, Artificial Sequence
  Description of Artificial Sequence Synthetic polypeptide
MTSSRLWFSLLLAAAFAGRATALWPWPQNFQTSDQRYVLYPNNFQFQYDVSSAAQPGCSVLDEAFQRYRDLLFGSGSWPRPYLTGKRHTLEKNVLVVSVVTPGCNQLPTLESVENYTLTINDDQCLLLSETVWGALRGLETFSQLVWKSAEGTFFINKTEIEDFPRFPHRGLLLDTSRHYLPLKSILDTLDVMAYNKLNVFHWHLVDDQSFPYESFTFPELMRKGSYSLSHIYTAQDVKEVIEYARLRGIRVLAEFDTPGHTLSWGPGIPGLLTPCYSGSEPSGTFGPVNPSLNNTYEFMSTFFLEVSSVFPDFYLHLGGDEVDFTCWKSNPEIQDFMRKKGFGEDFKQLESFYIQTLLDIVSSYGKGYVVWQEVFDNKVKIQPDTIIQVWREDIPVNYMKELELVTKAGFRALLSAPWYLNRISYGQDWRKFYKVEPLAFEGTPEQKALVIGGEACMWGEYVDATNLVPRLWPRAGAVAERLWSNKLTRDMDDAYDRLSHFRCELVRRGVAAQPLYAGYCNQEFEQT

SEQ ID NO: 7 - DNA, 1584 bp, Artificial Sequence
  Description of Artificial Sequence Synthetic polynucleotide
atgacttcctcccgcctttggttctccctcctgcttgccgctgccttcgccggacgcgccaccgccctgtggccgtggcctcagaacttccagactagcgaccaaagatacgtgctgtacccgaacaacttccagtttcaatacgacgtcagcagcgccgcccagcccggctgctcggtgctcgatgaggcattccagcggtaccgggatctcttgttcggttccggatcatggcctcggccgtacctcactggaaagaggcacactctcgaaaagaacgtcctggtggtgtccgtggtcacccctggctgcaatcagctgcccaccctggaatcagtggagaactacactttgaccatcaacgatgaccaatgcctgctgctgtcggagactgtgtggggtgccctgcgcgggctggaaacctttagccaactggtctggaagtcagccgaggggaccttcttcattaacaagaccgaaatcgaggacttccctcggttcccgcaccgcggcttgctgctggatacctcgcggcactatctgccactgaagtccattctcgataccctggacgtgatggcctacaacaagctgaacgtgttccactggcatctcgtggacgaccagtcctttccctacgagtccttcaccttccctgagttgatgagaaagggctcctactccctctcccatatctacacggctcaggacgtgaaggaagtgatcgaatatgcccggctgagagggattagggtgctggcagaatttgacaccccgggacacaccctgtcgtggggccctggtatcccgggcctgctgactccgtgctactccggctccgagccatccggaaccttcggacctgtgaatccctccctgaacaacacttacgagttcatgtccaccttcttcctggaagtgtcgagcgtgttccccgacttctacctccacctcggtggcgacgaagtcgatttcacttgctggaagtctaaccccgagatccaagatttcatgcgaaagaaaggattcggagaggactttaagcagctggagtccttctacatccaaaccctgctggacattgtgtcatcgtatggaaagggatacgtggtgtggcaggaagtgtttgacaataaggtcaaaattcagcccgatacaatcatccaagtctggcgcgaagatatccccgtgaactacatgaaagaactggaactggtcacgaaggctggattcagggcgcttctgagcgccccttggtacttgaaccggattagctacggccaggactggaggaagttctacaaggtcgaacctctggccttcgagggaacccccgagcagaaggccttggtgatcggcggcgaagcctgtatgtggggggaatacgtggacgcgaccaacctggtgccgcgcctgtggccgagagcgggagcagtggccgagcggctctggtcgaacaagctgactagggacatggacgatgcatacgacagactgagccacttccgctgcgaacttgtgcggcggggggtggcggcccagccgctctacgcggggtactgtaaccaggagttcgagcagacc

SEQ ID NO: 8 - DNA, 1683 bp, Artificial Sequence
  Description of Artificial Sequence Synthetic polynucleotide
atggaactttgcggactcggcctcccaagaccacctatgcttctcgccctgctgctcgccaccttgctcgcggctatgcttgcgctcctgactcaagtggcccttgtggtccaagtggccgaggctgcccgcgccccgagcgtgtcagccaagccaggaccggccctgtggccgctgcctctgagcgtgaagatgactcccaatctcctgcacctggccccggaaaacttctacatctcgcactcgccgaacagcaccgccggtccctcctgcaccctgctcgaagaggcattccggcggtaccacggatacatcttcggtttctataagtggcatcacgagccggcagagttccaggccaagactcaggtccagcagctgctcgtgtccattaccctgcaatcggagtgcgacgccttccccaacatcagctcagacgagtcatacactttgctcgtgaaggaacctgtcgccgtgctgaaggccaaccgcgtgtggggtgccctgcgcgggctggaaacctttagccaactggtctaccaagattcatacgggaccttcaccattaacgagtccaccatcatcgactcccctcggttcccgcaccgcggcttgctgctggatacctcgcggcactatctgccactgaagtccattctcgataccctggacgtgatggcctacaacaagctgaacgtgttccactggcatctcgtggacgaccagtcctttccctacgagtccttcaccttccctgagttgatgagaaagggctcctactccctctcccatatctacacggctcaggacgtgaaggaagtgatcgaatatgcccggctgagagggattagggtgctggcagaatttgacaccccgggacacaccctgtcgtggggccctggtatcccgggcctgctgactccgtgctactccggctccgagccatccggaaccttcggacctgtgaatccctccctgaacaacacttacgagttcatgtccaccttcttcctggaagtgtcgagcgtgttccccgacttctacctccacctcggtggcgacgaagtcgatttcacttgctggaagtctaaccccgagatccaagatttcatgcgaaagaaaggattcggagaggactttaagcagctggagtccttctacatccaaaccctgctggacattgtgtcatcgtatggaaagggatacgtggtgtggcaggaagtgtttgacaataaggtcaaaattcagcccgatacaatcatccaagtctggcgcgaagatatccccgtgaactacatgaaagaactggaactggtcacgaaggctggattcagggcgcttctgagcgccccttggtacttgaaccggattagctacggccaggactggaggaagttctacaaggtcgaacctctggccttcgagggaacccccgagcagaaggccttggtgatcggcggcgaagcctgtatgtggggggaatacgtggacgcgaccaacctggtgccgcgcctgtggccgagagcgggagcagtggccgagcggctctggtcgaacaagctgactagggacatggacgatgcatacgacagactgagccacttccgctgcgaacttgtgcggcggggggtggcggcccagccgctctacgcggggtactgtaaccaggagttcgagcagacc

SEQ ID NO: 9 - DNA, 1668 bp, Artificial Sequence
  Description of Artificial Sequence Synthetic polynucleotide
atggaactttgtggacttggactccctcgccctccgatgctgcttgcactgctcctcgccactctcctcgccgctatgctggcgctgctgactcaagtggcactggtggtccaagtggccgaggcggcccgcgcaccatccgtgtccgcgaagccgggcccagccctgtggcccctgcctctgagcgtgaagatgacccctaaccttctgcacctggcgccggagaacttctacataagccactcgcccaactcgaccgcgggtccctcctgtaccctgctggaggaggccttcaggcgctaccacggctacattttcgggttctataagtggcaccacgagccggccgagttccaagccaagactcaggtccaacagctgctggtgtcaatcacactgcagtccgaatgcgatgctttccctaatatctcctccgacgagtcctacaccctgctcgtgaaggaacctgtggccgtgctcaaggccaaccgcgtctggggagccctcaggggtctggaaaccttcagccagctggtgtaccaggacagctacggcaccttcaccattaacgagtccaccatcatcgactcaccgagattcagccaccggggaatcctgattgatacttcaagacactatttgcccgtgaagatcatcctgaaaaccctcgacgctatggcctttaacaagtttaacgtgttgcattggcatatcgtggacgatcagtccttcccgtatcaaagcatcacttttccggaactgtccaacaagggatcttactcgctgagccacgtgtacactcccaatgacgtgcgcatggtcatcgaatacgcccggctgaggggcattagagtgctccctgaatttgacacccccggccataccctctcctggggaaaaggacagaaagatttgctcacaccttgctactccggctccgagccctctggaactttcggccccatcaacccgaccctgaacaccacttactccttcttgaccacgttcttcaaggagatttccgaggtgttcccggaccagttcatccacctggggggcgacgaagtggagttcaagtgctgggaatcgaacccaaagatccaggacttcatgaggcagaaggggtttggcaccgatttcaagaagctggagtcgttctacattcaaaaggtcctggacatcattgcaaccatcaacaaggggtcaatcgtgtggcaggaagtgttcgatgacaaggctaagctggcgcccggcactattgtggaagtctggaaagactccgcctaccctgaggagctgtcgagagtcaccgcctccggattccccgtcatcctgagcgctccgtggtacctgaaccggatctcctacggacaagattggcgcaagtactacaaggtcgaaccgctggacttcggagggactcagaagcagaagcagctctttattggtggagaggcctgcctttggggcgaatacgtggacgccaccaacctcaccccgcggctctggccacgggccagcgccgtgggagaacgcctgtggtcgtcaaaggacgtccgcgatatggacgatgcctacgaccggctcactcggcatcggtgccggatggtggaacgcggtatcgcagcgcagccactgtacgctggatactgcaaccacgagaatatg

SEQ ID NO: 10 - protein, 556 aa, Artificial Sequence
  Description of Artificial Sequence Synthetic polypeptide
MELCGLGLPRPPMLLALLLATLLAAMLALLTQVALVVQVAEAARAPSVSAKPGPALWPLPLSVKMTPNLLHLAPENFYISHSPNSTAGPSCTLLEEAFRRYHGYIFGFYKWHHEPAEFQAKTQVQQLLVSITLQSECDAFPNISSDESYTLLVKEPVAVLKANRVWGALRGLETFSQLVYQDSYGTFTINESTIIDSPRFSHRGILIDTSRHYLPVKIILKTLDAMAFNKFNVLHWHIVDDQSFPYQSITFPELSNKGSYSLSHVYTPNDVRMVIEYARLRGIRVLPEFDTPGHTLSWGKGQKDLLTPCYSGSEPSGTFGPINPTLNTTYSFLTTFFKEISEVFPDQFIHLGGDEVEFKCWESNPKIQDFMRQKGFGTDFKKLESFYIQKVLDIIATINKGSIVWQEVFDDKAKLAPGTIVEVWKDSAYPEELSRVTASGFPVILSAPWYLNRISYGQDWRKYYKVEPLDFGGTQKQKQLFIGGEACLWGEYVDATNLTPRLWPRASAVGERLWSSKDVRDMDDAYDRLTRHRCRMVERGIAAQPLYAGYCNHENM

SEQ ID NO: 11 - protein, 339 aa, Artificial Sequence
  Description of Artificial Sequence Synthetic polypeptide
KSILDTLDVMAYNKLNVFHWHLVDDQSFPYESFTFPELMRKGSYSLSHIYTAQDVKEVIEYARLRGIRVLAEFDTPGHTLSWGPGIPGLLTPCYSGSEPSGTFGPVNPSLNNTYEFMSTFFLEVSSVFPDFYLHLGGDEVDFTCWKSNPEIQDFMRKKGFGEDFKQLESFYIQTLLDIVSSYGKGYVVWQEVFDNKVKIQPDTIIQVWREDIPVNYMKELELVTKAGFRALLSAPWYLNRISYGQDWRKFYKVEPLAFEGTPEQKALVIGGEACMWGEYVDATNLVPRLWPRAGAVAERLWSNKLTRDMDDAYDRLSHFRCELVRRGVAAQPLYAGYCN

SEQ ID NO: 12 - protein, 155 aa, Artificial Sequence
  Description of Artificial Sequence Synthetic polypeptide
ARAPSVSAKPGPALWPLPLSVKMTPNLLHLAPENFYISHSPNSTAGPSCTLLEEAFRRYHGYIFGFYKWHHEPAEFQAKTQVQQLLVSITLQSECDAFPNISSDESYTLLVKEPVAVLKANRVWGALRGLETFSQLVYQDSYGTFTINESTIIDS

SEQ ID NO: 13 - protein, 197 aa, Artificial Sequence
  Description of Artificial Sequence Synthetic polypeptide
MELCGLGLPRPPMLLALLLATLLAAMLALLTQVALVVQVAEAARAPSVSAKPGPALWPLPLSVKMTPNLLHLAPENFYISHSPNSTAGPSCTLLEEAFRRYHGYIFGFYKWHHEPAEFQAKTQVQQLLVSITLQSECDAFPNISSDESYTLLVKEPVAVLKANRVWGALRGLETFSQLVYQDSYGTFTINESTIIDS

SEQ ID NO: 14 - protein, 3 aa, Artificial Sequence
  Description of Artificial Sequence Synthetic peptide
  MOD_RES (2)..(2): Any amino acid
  MOD_RES (3)..(3): Ser or Thr
NXX

SEQ ID NO: 15 - protein, 15 aa, Artificial Sequence
  Description of Artificial Sequence Synthetic peptide
GSYSLSHIYTAQDVK

SEQ ID NO: 16 - protein, 11 aa, Artificial Sequence
  Description of Artificial Sequence Synthetic peptide
IQPDTIIQVWR

SEQ ID NO: 17 - protein, 8 aa, Artificial Sequence
  Description of Artificial Sequence Synthetic peptide
ISYGQDWR

SEQ ID NO: 18 - protein, 6 aa, Artificial Sequence
  Description of Artificial Sequence Synthetic 6xHis tag
HHHHHH

SEQ ID NO: 19 - protein, 4 aa, Artificial Sequence
  Description of Artificial Sequence Synthetic peptide
NPVT